(19) United States
(12) Patent Application Publication
(10) Pub. No.: US 2020/0079808 A1
(43) Pub. Date: Mar. 12, 2020

(54) GLYCOPOLYMERS SEQUESTERING CARBOHYDRATE-BINDING PROTEINS

(71) Applicant: POLYNEURON PHARMACEUTICALS AG, Basel (CH)

(72) Inventors: Hélène Blanche PFISTER, Basel (CH); Ruben HERRENDORFF, Basel (CH); Beat ERNST, Basel (CH)

(21) Appl. No.: 16/493,959

(22) PCT Filed: Mar. 15, 2018
(86) PCT No.: PCT/EP2018/056583

§ 371 Date: Sep. 13, 2019

(30) Foreign Application Priority Data
Mar. 15, 2017 (EP) 17161162.7

(51) Int. Cl. C07H 15/12; A61K 47/64; C07H 15/04; C07H 15/203

(52) U.S. Cl. CPC C07H 15/12; C07H 15/203; C07H 15/04; A61K 47/645

(57) ABSTRACT

The invention relates to polymers comprising carbohydrate ligands and moieties, respectively, that bind to carbohydrate-binding proteins (CBPs), as well as to these carbohydrate ligands, and to their use in diagnosis and therapy of diseases that are associated with CBP-mediated cytotoxicity, agglutinatination, or immune complex deposit formation. In particular, the invention relates to polymers comprising a multitude of said carbohydrate ligands and moieties, respectively, mimicking carbohydrates that are bound by CBPs which belong to the group of (i) bacterial exotoxins, (ii) agglutinins, and (iii) immune complex deposit-forming immunoglobulins. Furthermore, the invention relates to the use of these polymers and carbohydrate ligands and moieties respectively, in diagnosis as well as for the treatment of diseases that are associated with CBP-mediated cytotoxicity, agglutinatination, or immune complex deposit formation. In one embodiment, the polymer is polylysine.

### FIELD OF THE INVENTION

The invention relates to carbohydrate ligands and moieties, respectively, that bind to carbohydrate-binding proteins (CBPs), polymers comprising these carbohydrate ligands, and to their use in diagnosis and therapy of diseases that are associated with CBP-mediated cytotoxicity, agglutinatination, or immune complex deposit formation.

### BACKGROUND OF THE INVENTION

Carbohydrate-binding proteins (CBPs) are characterized by selective binding of specific carbohydrate structures. CBPs are ubiquitous, thus can be found in humans, animals, microbes, plants, and fungi, where they promote surface-interactions (e.g. cell-cell, cell-matrix, cell-macromolecule, macromolecule-macromolecule interactions). CBPs generally promote adhesion functions but can also be involved in signaling functions. Via their carbohydrate-recognition domains (CRDs) they decipher the glycocode that is composed by the broad diversity of carbohydrates that cover cells (glycocalyx), a substantial number of macromolecules (glycosylation), or that are present in the extracellular matrix. A common caracteristic of CBP-carbohydrate binary interactions is the low binding affinity, usually in the micromolar range, and short dissociative half-lifes, usually in the range of seconds. Low affinity and short dissociative half-lifes of binary CBP-carbohydrate complexes is often overcome by multivalent interaction. Carbohydrates and CBPs play critical roles in physiological but also in pathological conditions. (Holgersson et al., Immunol Cell Biol, 2005, 83, 694-708; B. Ernst and J. Magnani, 2009, 8, 661-677)

Three particularly disease-relevant types of CBPs are (i) bacterial exotoxins, (ii) agluttinins, and (iii) immune complex deposit-forming immunoglobulins.

(i) Bacterial Exotoxins

Severe infections are caused by bacteria that secrete CBPs that function as bacterial exotoxins. The CBPs interact with host cell surface carbohydrates and thereby promote toxin-attachment. The attachment event is usually followed by a mechanism leading to cytotoxicity and increased virulence in host cells. Examples for such bacterial exotoxins with carbohydrate-binding properties are Shiga toxin (Shigella dysenteriae and other Shigella strains), Shiga-like toxin/vero toxin (Escherichia coli), cholera toxin (Vibrio cholerae), heat-labile enterotoxin (enterotoxigenic Escherichia coli), toxin A (Clostridium difficile), botulinum toxin (Clostridium botulinum), tetanus toxin (Clostridium tetani), and the pertussis toxin secreted by Bordetella pertussis. All of these toxins belong to the group of AB toxins, i.e. the heterohexameric AB₅ toxins (Shiga toxin, Shiga-like toxin/vero toxin, cholera toxin, heat-labile enterotoxin, pertussis toxin), furthermore the binary AB toxins (tetanus toxin, botulinum toxin, toxin A). The A subunit is responsible for an enzymatic function leading to host cell damage or destruction whereas the B subunit is responsible for carbohydrate receptor binding on the host cell surface and subsequent toxin internalization into the host cell (J. W. Wilson, Postgrad Med J, 2002, 78, 216-224; J. D. Esko, N. Sharon, Microbial Lectins: Hemagglutinins, Adhesins, and Toxins. In: A. Varki, R. D. Cummings, J. D. Esko, et al., editors. Essentials of Glycobiology. 2nd edition. Cold Spring Harbor (NY): Cold Spring Harbor Laboratory Press; 2009. Chapter 34. Available from: httbs://ncbi.nlm.nih.govibooks/NBK1907/).

Carbohydrates structures bound by the different exotoxin B subunits are e.g. the GM1 ganglioside (cholera toxin and heat-labile enterotoxin) the Gb3 glycolipid (Shiga toxin), the GT1b and GQ1b gangliosides (botulinum toxin), and the GT1b ganglioside (tetanus toxin) (J. D. Esko, N. Sharon, Microbial Lectins: Hemagglutinins, Adhesins, and Toxins. In: A. Varki, R. D. Cummings, J. D. Esko, et al., editors. Essentials of Glycobiology. 2nd edition. Cold Spring Harbor (NY): Cold Spring Harbor Laboratory Press; 2009. Chapter 34. Available from: https://www.ncbi.nlm.nih.govibooks/NBK1907/). The pertussis toxin B subunit recognizes Neu5Ac(α2-6)Gal(β1-4)GlcNAc and Neu5Ac(α2-3)Gal(β1-4)GlcNAc containing oligosaccharides (S. H. Millen et al., Biochemistry, 2010, 49(28), 5954-5967) whereas the Chlostridium difficile toxin A recognizes the linear-B-trisaccharide Gal(α1-3)Gal(β1-4)GlcNAc but also the structurally related Gal(α1-4β)GlcNAc containing Lewis antigens, e.g. the Lewis X and Y antigens (C.-Y. Yeh et al., Infect Immun, 2008, 76(3), 1170-1178).

(ii) AGluttinins

Agluttinins are immunoglobulins that bind carbohydrate antigens on red blood cells and thereby cause agglutination of red blood cells in patients. Such an agglutination can be of an autoimmune etiology or the cause of an incompatible transplantation/transfusion. The most relevant carbohydrates bound by agglutinins are part of the ABH system, the I and the P system. Agglutinins cause different disorders such as cold agglutinin disease (CAD) which is associated with anti-I system agglutinin or paroxysmal cold hemoglobinuria (PCH) which is associated with anti-P system agglutinin (S. Berentsen and T. Sundic, Transfus Med Hemother, 2015, 42(5): 303-310). Incompatability complications in transplantations or transfusions are often related to incompatability in the ABH carbohydrate antigen system and mediated by anti-A and anti-B agluttinins (Holgersson et al., Immunol Cell Biol, 2005, 83, 694-708). Agglutinins directed against the Tn (or sialyl-Tn) carbohydrate antigen are associated with a disorder called Tn-polyagglutination syndrome, a disorder characterized by the agglutination of erythrocytes by immunoglobulins binding the Tn-antigen (GalNAc) and sialyl-Tn (Neu5Ac(α2-6)GalNAc) on erythrocytes. This antigen is exposed on the erythrocyte surface only under pathological conditions and is linked to impaired T-synthetase (C1GALT1) activity in this syndrome (V. K. Crew et al., Br J Haematol, 2008, 142(4):657-667.

(iii) Immune Complex Forming Immunoglobulins

Disabling disorders are caused by immune complex formation by immunoglobulins raised against carbohydrate antigens on other immunoglobulins. The formed immune complexes are deposited in tissues such as the kidney where they cause inflammation and tissue damage. IgA nephropathy (also known as IgA nephritis or Berger disease or synpharyngitic glomerulonephritis) and IgA vasculitis (also known as Henoch Schönlein Purpura HSP) are examples for such disorders and are characterized by mesangial IgA immune complex deposits in the glomeruli. Both disorders are linked to immunoglobulins recognizing misglycosylated, galactose-deficient IgA1 immunoglobulins where the Tn-antigen (GalNAc) and sialyl-Tn (Neu5Ac(α2-6)GalNAc) antigen are exposed under pathophysiological conditions. The immunoglobulins that recognize the Tn or sialyl Tn antigen are mostly of the IgG or IgA isotype, but can also be of the IgM isotype (B. Knoppova et al., Front Immunol, 2016, 7, 117).

### SUMMARY OF THE INVENTION

The invention relates to polymers comprising carbohydrate ligands and moieties, respectively, that bind to carbohydrate-binding proteins (CBPs), as well as to these carbohydrate ligands, and to their use in diagnosis and therapy of diseases that are associated with CBP-mediated cytotoxicity, agglutinatination, or immune complex deposit formation. In particular, the invention relates to polymers comprising a multitude of said carbohydrate ligands and moieties, respectively, mimicking carbohydrates that are bound by CBPs which belong to the group of (i) bacterial exotoxins, (ii) agglutinins, and (iii) immune complex deposit-forming immunoglobulins. Furthermore, the invention relates to the use of these polymers and carbohydrate ligands and moieties respectively, in diagnosis as well as for the treatment of diseases that are associated with CBP-mediated cytotoxicity, agglutinatination, or immune complex deposit formation.

Thus, the present invention, in particular, provides for a polymer comprising a multitude of a compound, wherein said compound comprises a carbohydrate moiety and a linker Z, and wherein said carbohydrate moiety mimics, or alternatively and preferably is, a glycoepitope that is bound by, CBPs with cytotoxic, agglutinating or immune complex deposit-forming properties.

The polymers, compounds and compositions of the present invention, therefore, provide for new treatments of diseases and disorders associated with and caused by bacterial exotoxins, agglutinins, and immune complex deposit-forming immunoglobulins by selective in vivo neutralization and sequestration and removal, respectively, of these bacterial exotoxins, agglutinins, and immune complex deposit-forming immunoglobulins by using said inventive polymers, compounds and compositions, in particular by using said, preferably biodegradable, polymers of the present invention.

In particular, blocking the adhesion of the exotoxin B subunits to the host cell surface carbohydrates with the inventive polymers, compounds and compositions allows the treatment of infections caused by e.g. Shigella dysenteriae, and thus treatment of shigellosis, bacillary dysentery, Marlow syndrome and hemolytic-uremic syndrome (HUS), by (enterotoxigenic) Escherichia coli, and thus treatment of travelers' diarrhea, by Vibrio cholerae, and thus treatment of cholera, by Clostridium difficile, by Clostridium botulinum, and thus treatment of botulinism, by Clostridium tetani, and thus treatment of tetanus, and by Bordetella pertussis, and thus treatment of pertussis or whooping cough.

Thus, in a first aspect, the present invention provides for a polymer comprising a multitude of a compound, wherein said compound comprises a carbohydrate moiety and a linker Z, and wherein said carbohydrate moiety mimics a glycoepitope recognized by a carbohydrate-binding protein (CBP), wherein said CBP is selected from a bacterial exotoxin, an agluttinin and an immune complex deposit-forming immunoglobulin, and wherein said linker Z is —X-A-(B)ₚ-(CH₂)_{q}—Y, wherein
X is O or N(R^{a});
R^{a} is H, C₁₋₄-alkyl, C₁₋₄alkoxy, CH₂C₆H₅, CH₂CH₂C₆H₅, OCH₂C₆H₅, or OCH₂CH₂C₆H₅;
A is C₁₋₇alkylene, OC₁₋₇alkylene, C₁₋₄alkylene-(OCH₂CH₂)ᵣOC₁₋₄alkylene, OC₁₋₇alkylene-R^{b}, or R^{b}—C₁₋₇alkylene wherein R^{b} is an optionally substituted aryl or an optionally substituted heteroaryl, and wherein r is 0 to 6, preferably r is 1, 2 or 3, and further preferably r is 1;
B is NHC(O) or S;
p is 0 or 1;
q is 0 to 6, preferably q is 0 to 4, and further preferably q is 0, 2 or 3;
Y is SH, N₃ or NH₂; and

wherein said linker Z is covalently bound via its —X-group to the reducing end of said carbohydrate moiety; and wherein said multitude of said compound is connected to the polymer backbone by way of said linker Z, and wherein said connection is effected via the Y-group of said linker Z; and

wherein said compound is not

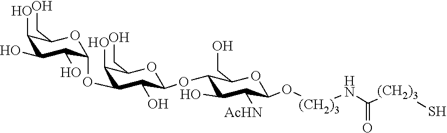

when said polymer backbone is poly-L-lysine.

In a further aspect, the present invention provides for a polymer comprising a multitude of a compound, wherein said compound comprises a carbohydrate moiety and a linker Z, and wherein said carbohydrate moiety mimics a glycoepitope recognized by a carbohydrate-binding protein (CBP), wherein said CBP is selected from a bacterial exotoxin, an agluttinin and an immune complex deposit-forming immunoglobulin, and wherein said linker Z is —X-A-(B)ₚ-(CH₂)_{q}—Y, wherein
X is O or N(R^{a});
R^{a} is H, C₁₋₄-alkyl, C₁₋₄alkoxy, CH₂C₆H₅, CH₂CH₂C₆H₅, OCH₂C₆H₅, or OCH₂CH₂O₆H₅;
A is C₁₋₇alkylene, OC₁₋₇alkylene, C₁₋₄alkylene-(OCH₂CH₂)ᵣOC₁₋₄alkylene, OC₁₋₇alkylene-R^{b}, or R^{b}—C₁₋₇alkylene wherein R^{b} is an optionally substituted aryl or an optionally substituted heteroaryl, and wherein r is 0 to 6, preferably r is 1, 2 or 3, and further preferably r is 1;
B is NHC(O) or S;
p is 0 or 1;
q is 0 to 6, preferably q is 0 to 4, and further preferably q is 0, 2 or 3;
Y is SH, N₃ or NH₂; and

wherein said linker Z is covalently bound via its —X-group to the reducing end of said carbohydrate moiety; and wherein said multitude of said compound is connected to the polymer backbone by way of said linker Z, and wherein said connection is effected via the Y-group of said linker Z converting said Y-group of said linker Z to *—S—, a triazolyl-moiety or *—NH—, wherein said triazolyl-moiety is preferably

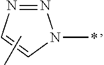

wherein *— corresponds to the binding to the (CH₂)_{q}-moiety of said linker Z, and wherein — corresponds to the connection of said *—S—, a triazolyl-moiety or *—NH— to the polymer backbone; and

wherein said compound is not

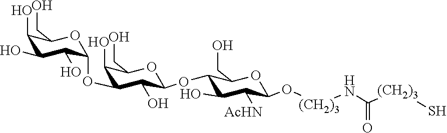

when said polymer backbone is poly-L-lysine.

In a further aspect, the present invention provides for a polymer comprising a multitude of a compound, wherein said compound comprises a carbohydrate moiety and a linker Z, and wherein said carbohydrate moiety mimics a glycoepitope recognized by a carbohydrate-binding protein (CBP), wherein said CBP is selected from a bacterial exotoxin, an agluttinin and an immune complex deposit-forming immunoglobulin, and wherein said linker Z is —X-A-(B)ₚ-(CH₂)_{q}-Y, wherein
X is O or N(R^{a});
R^{a} is H, C₁₋₄-alkyl, C₁₋₄alkoxy, CH₂C₆H₅, CH₂CH₂C₆H₅, OCH₂C₆H₅, or OCH₂CH₂C₆H₅;
A is C₁₋₇alkylene, OC₁₋₇alkylene, C₁₋₄alkylene-(OCH₂CH₂)ᵣOC₁₋₄alkylene, OC₁₋₇alkylene-R^{b}, or R^{b}-C₁₋₇alkylene wherein R^{b} is an optionally substituted aryl or an optionally substituted heteroaryl, and wherein r is 0 to 6, preferably r is 1, 2 or 3, and further preferably r is 1;
B is NHC(O) or S;
p is 0 or 1;
q is 0 to 6, preferably q is 0 to 4, and further preferably q is 0, 2 or 3;
Y is SH, N₃ or NH₂; and

wherein said linker Z is covalently bound via its —X-group to the reducing end of said carbohydrate moiety; and wherein said multitude of said compound is connected to the polymer backbone by way of said linker Z, and wherein said connection is effected via the Y-group of said linker Z; and

wherein said compound is not

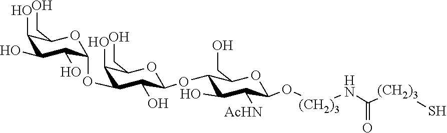

when said polymer backbone is poly-L-lysine; and

wherein said compound is not a compound comprising a carbohydrate moiety and a linker Z, wherein said carbohydrate moiety mimics a glycoepitope comprised by a glycosphingolipid of the nervous system, wherein said linker Z is —N(R^{a})-A-B-CH₂—(CH₂)_{q}—SH, wherein R^{a} is H, C₁₋₄alkyl, C₁-C₄-alkoxy, CH₂C₆H₅, CH₂CH₂C₆H₅, OCH₂C₆H₅, or OCH₂CH₂C₆H₅; A is C₁₋₇alkylene, C₁-C₇-alkoxy, C₁₋₄alkyl-(OCH₂CH₂)ₚO—C₁₋₄alkyl, or C₁-C₇-alkoxy-R^{b}, wherein R^{b} is an optionally substituted aryl or an optionally substituted heteroaryl, and wherein p is 0 to 6, preferably p is 1, 2 or 3, and further preferably p is 1; B is NHC(O), S or CH₂; q is 0 to 6, preferably q is 1, 2, 3 or 4, and further preferably q is 1 or 2; and wherein said linker Z is covalently bound via its —N(R^{a})-group to the reducing end of said carbohydrate moiety.

In a further aspect, the present invention provides for a polymer comprising a multitude of a compound, wherein said compound comprises a carbohydrate moiety and a linker Z, and wherein said carbohydrate moiety mimics a glycoepitope recognized by a carbohydrate-binding protein (CBP), wherein said CBP is selected from a bacterial exotoxin, an agluttinin and an immune complex deposit-forming immunoglobulin, and wherein said linker Z is —X-A-(B)ₚ-(CH₂)_{q}—Y, wherein
X is O or N(R^{a});
R^{a} is H, C₁₋₄-alkyl, C₁₋₄alkoxy, CH₂C₆H₅, CH₂CH₂C₆H₅, OCH₂C₆H₅, or OCH₂CH₂C₆H₅;
A is C₁₋₇alkylene, OC₁₋₇alkylene, C₁₋₄alkylene-(OCH₂CH₂)ᵣOC₁₋₄alkylene, OC₁₋₇alkylene-R^{b}, or R^{b}—C₁₋₇alkylene wherein R^{b} is an optionally substituted aryl or an optionally substituted heteroaryl, and wherein r is 0 to 6, preferably r is 1, 2 or 3, and further preferably r is 1;
B is NHC(O) or S;
p is 0 or 1;
q is 0 to 6, preferably q is 0 to 4, and further preferably q is 0, 2 or 3;
Y is SH, N₃ or NH₂; and

wherein said linker Z is covalently bound via its —X-group to the reducing end of said carbohydrate moiety; and wherein said multitude of said compound is connected to the polymer backbone by way of said linker Z, and wherein said connection is effected via the Y-group of said linker Z converting said Y-group of said linker Z to *—S—, a triazolyl-moiety or *—NH—, wherein said triazolyl-moiety is preferably

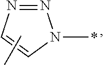

wherein *— corresponds to the binding to the (CH₂)_{q}-moiety of said linker Z, and wherein — corresponds to the connection of said *—S—, a triazolyl-moiety or *—NH— to the polymer backbone; and

wherein said compound is not

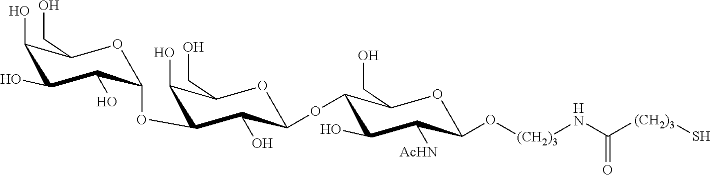

when said polymer backbone is poly-L-lysine; and

wherein said compound is not a compound comprising a carbohydrate moiety and a linker Z, wherein said carbohydrate moiety mimics a glycoepitope comprised by a glycosphingolipid of the nervous system, wherein said linker Z is —N(R^{a})-A-B-CH₂—(CH₂)_{q}—SH, wherein R^{a} is H, C₁₋₄alkyl, C₁-C₄-alkoxy, CH₂C₆H₅, CH₂CH₂C₆H₅, OCH₂C₆H₅, or OCH₂CH₂C₆H₅; A is C₁₋₇alkylene, C₁-C₇-alkoxy, C₁₋₄alkyl-(OCH₂CH₂)ₚO—C₁₋₄alkyl, or C₁-C₇-alkoxy-R^{b}, wherein R^{b} is an optionally substituted aryl or an optionally substituted heteroaryl, and wherein p is 0 to 6, preferably p is 1, 2 or 3, and further preferably p is 1; B is NHC(O), S or CH₂; q is 0 to 6, preferably q is 1, 2, 3 or 4, and further preferably q is 1 or 2; and wherein said linker Z is covalently bound via its —N(R^{a})-group to the reducing end of said carbohydrate moiety.

In the present invention, and in particular, when referring to any and all aspects and any and all embodiments of the inventive polymers or its uses, and when hereby referring to the Y-group of the linker Z being SH, N₃ or NH₂, it is to be understood that said Y-group of said linker Z within said polymers is present as *—S—, a triazolyl-moiety or *—NH—, wherein said triazolyl-moiety is preferably

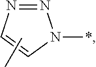

wherein *— corresponds to the binding to the (CH₂)_{q}-moiety of said linker Z, and wherein - corresponds to the connection of said *—S—, a triazolyl-moiety or *—NH— to the polymer backbone.

Said triazolyl-moiety being preferably said

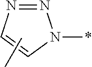

is selected from (i)

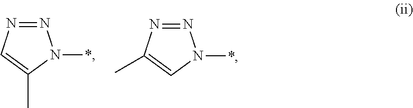

or a mixture of (i) and (ii) in any ratio, further preferably

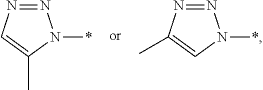

and again further preferably

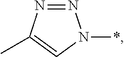

wherein *— corresponds to the binding to the (CH₂)_{q}-moiety of said linker Z, and wherein — corresponds to the connection of said triazolyl-moiety to the polymer backbone.

In a further aspect, the present invention provides for a polymer comprising a multitude of a compound, wherein said compound comprises a carbohydrate moiety and a linker Z, and said compound is a compound of formula (I), formula (II), formula (III) or formula (IV), wherein formula (I) is

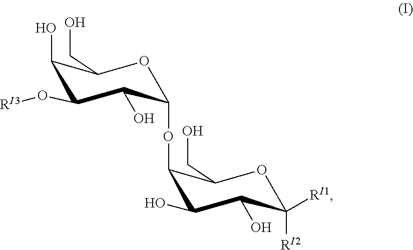

wherein R^{I1} is H or Z or

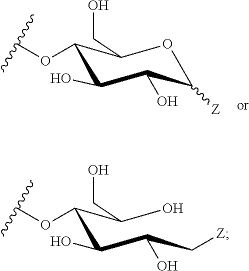

wherein R^{I2} is H or Z;
   wherein, when R^{I1} is H, then R^{I2} is Z; and when R^{I1} is not H, then R^{I2} is H; and thus, R^{I1} and R^{I2} cannot be both H and R^{I1} and R^{I2} cannot be both Z;
and wherein R^{I3} is H or

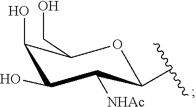

wherein formula (II) is

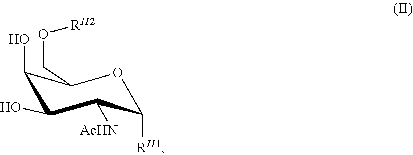

wherein R^{II1} is Z or

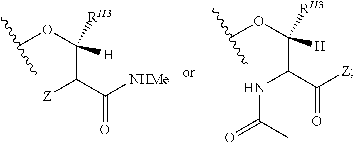

wherein R^{II2} is H or

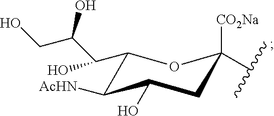

and wherein R^{II3} is H or Me;

wherein formula (III) is

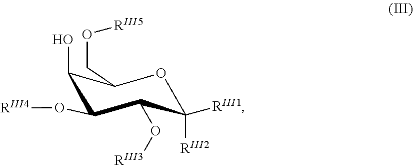

wherein R^{II3} is H or Z or

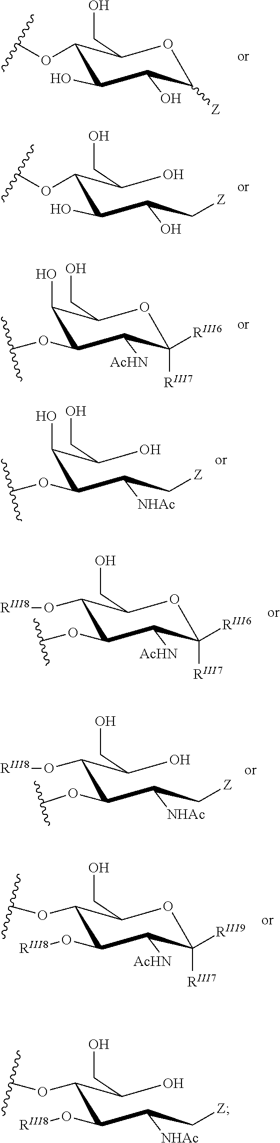

wherein R^{III2} is H or Z;
   wherein when R^{III1} is H, then R^{III2} is Z; and when R^{III1} is not H, then R^{III2} is H;
   and thus, R^{III1} and R^{III2} cannot be both H and R^{III1} and R^{III2} cannot be both Z;
wherein R^{III3} and R^{III8} are independently H or

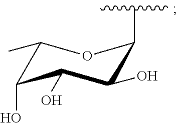

wherein when R^{III1} is not

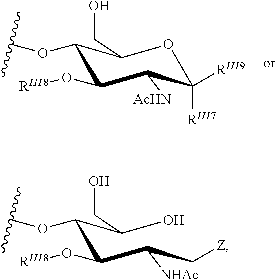

then R^{III3} is H;
wherein R^{III4} is H or

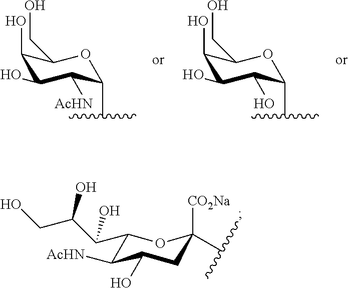

wherein when R^{III4} is not H, then R^{III8} is H;
wherein R^{III3} is H, then R^{III4} is H
wherein R^{III5} is H or

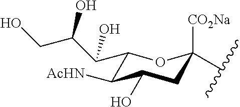

wherein when R^{III4} or R^{III5} is

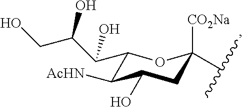

then R^{III1} is H, Z or

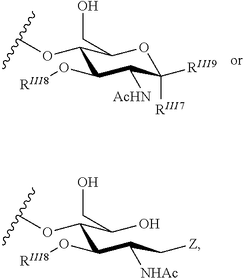

and R^{III3} and R^{III8} are H;
wherein R^{III6} is H or Z or

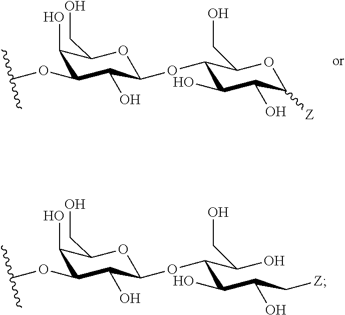

wherein R^{III7} is H or Z;
   wherein when R^{III6} is H, then R^{III7} is Z and when R^{III6} is not H, then R^{III7} is H;
   and thus, R^{III6} and R^{III7} cannot be both H and R^{III6} and R^{III7} cannot be both Z;
wherein R^{III9} is H or Z or

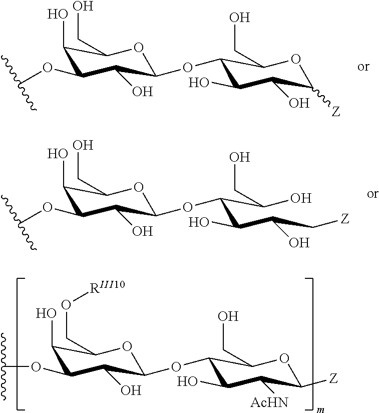

wherein m is 1 to 3;
   wherein when R^{III9} is

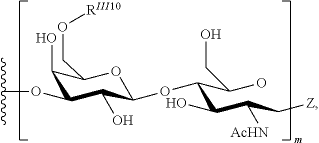

then R^{III3}, R^{III4}, R^{III5} and R^{III8} are H;
wherein R^{III10} is H or

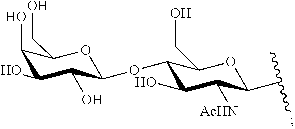

wherein formula (IV) is

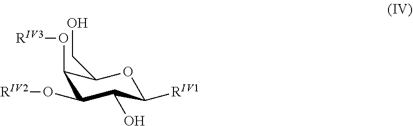

wherein R^{IV1} is

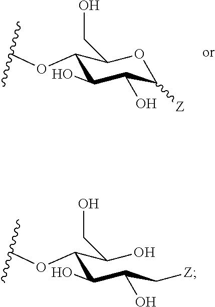

wherein R^{IV2} and R^{IV4} are independently H or

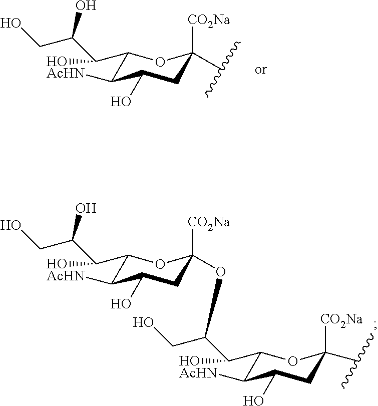

wherein R^{IV3} is H or

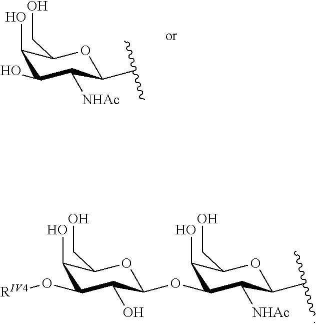

In a further aspect, the present invention provides for a polymer comprising a multitude of a compound, wherein said compound comprises a carbohydrate moiety and a linker Z, and said compound is a compound of formula (I), formula (II), formula (III) or formula (IV), wherein formula (I) is

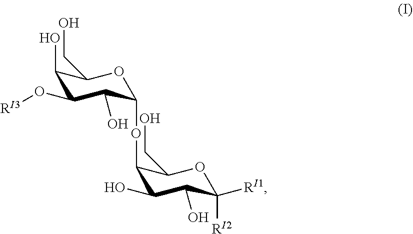

wherein R^{I1} is H or Z or

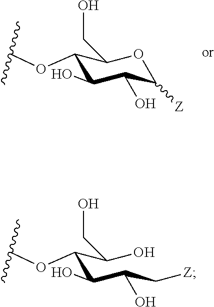

wherein R^{I2} is H or Z;
   wherein, when R^{I1} is H, then R^{I2} is Z; and when R^{I1} is not H, then R^{I2} is H; and
   thus, R^{I1} and R^{I2} cannot be both H and R^{I1} and R^{I2} cannot be both Z;
and wherein R^{I3} is H or

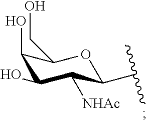

wherein formula (II) is

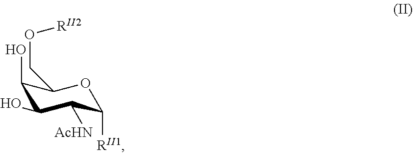

wherein R^{II1} is Z or

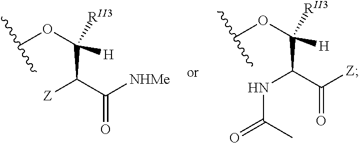

wherein R^{II2} is H or

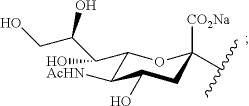

and wherein R^{II3} is H or Me;

wherein formula (III) is

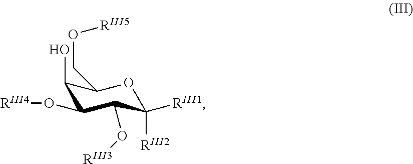

wherein R^{III1} is H or Z or

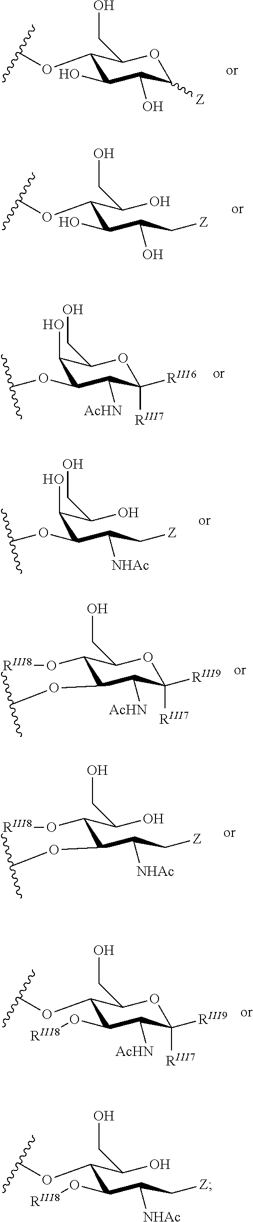

wherein R^{III2} is H or Z;
   wherein when R^{III1} is H, then R^{III2} is Z; and when R^{III1} is not H, then R^{III2} is H;
   and thus, R^{III1} and R^{III2} cannot be both H and R^{III1} and R^{III2} cannot be both Z;
wherein R^{III3} and R^{III8} are independently H or

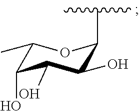

wherein when R^{III1} is not

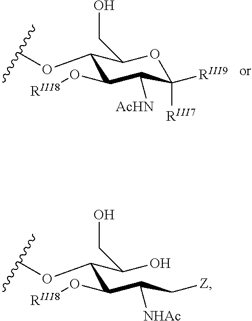

then R^{III3} is H;
wherein R^{III4} is H or

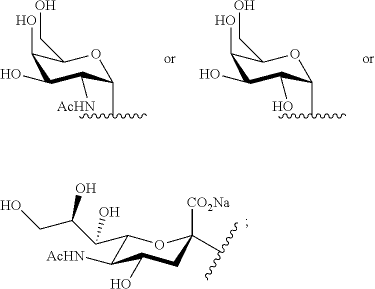

wherein when R^{III4} is not H, then R^{III8} is H;
wherein R^{III3} is H, then R^{III4} is H
wherein R^{III5} is H or

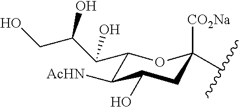

wherein when R^{III4} or R^{III5} is

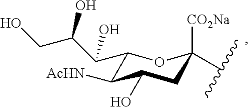

then R^{III1} is H, Z or

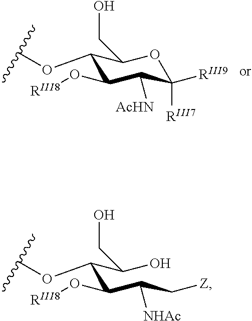

and R^{III3} and R^{III8} are H;
wherein R^{III6} is H or Z or

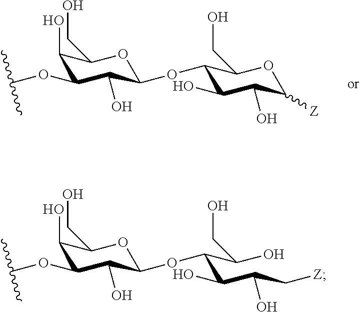

wherein R^{III7} is H or Z;
   wherein when R^{III6} is H, then R^{III7} is Z and when R^{III6} is not H, then R^{III7} is H;
   and thus, R^{III6} and R^{III7} cannot be both H and R^{III6} and R^{III7} cannot be both Z;
wherein R^{III9} is H or Z or

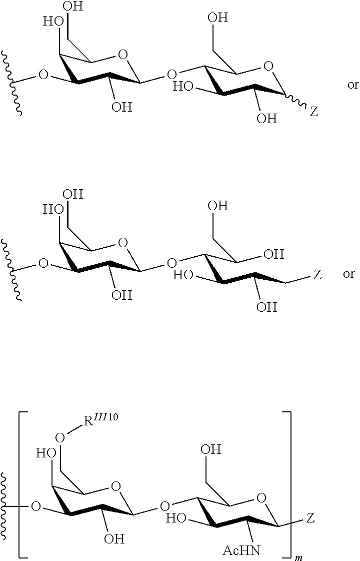

wherein m is 1 to 3;
   wherein when R^{III9} is

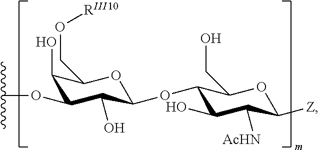

then R^{III3}, R^{III4}, R^{III5} and R^{III8} are H;
wherein R^{III10} is H or

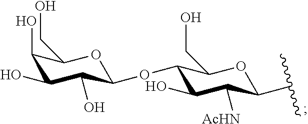

wherein formula (IV) is

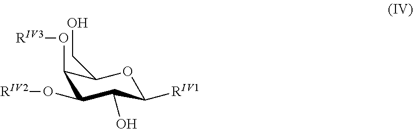

wherein R^{IV1} is

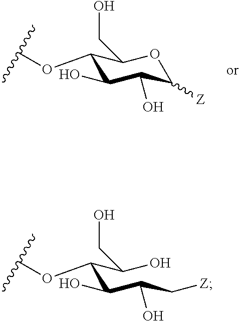

wherein R^{IV2} and R^{IV4} are independently H or

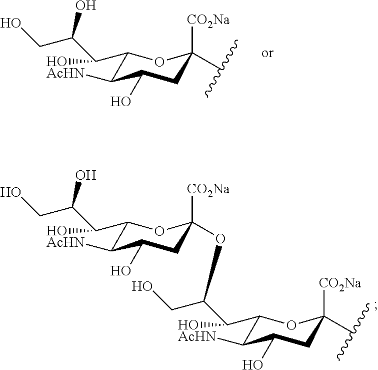

wherein R^{IV3} is H or

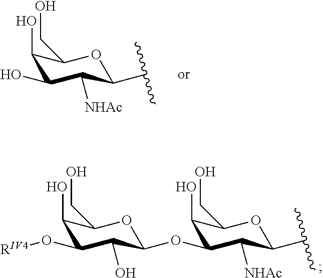

wherein when said compound is a compound of formula (IV), then said linker Z is not —N(R^{a})-A-B-CH₂—(CH₂)_{q}—SH, wherein R^{a} is H, C₁-C₄-alkyl, C₁-C₄-alkoxy, CH₂C₆H₅, CH₂CH₂C₆H₅, OCH₂C₆H₅, or OCH₂CH₂C₆H₅; A is C₁₋₇alkylene, C₁-C₇-alkoxy, C₁₋₄alkylene-(OCH₂CH₂)ᵣO—C₁₋₄alkylene or C₁-C₇alkoxy-R^{b}, wherein R^{b} is an optionally substituted aryl or an optionally substituted heteroaryl, and wherein r is 0 to 6, preferably r is 1, 2 or 3, and further preferably r is 1; B is NHC(O), S or CH₂; q is 0 to 6, preferably q is 1, 2, 3 or 4, and further preferably q is 1 or 2.

In a further aspect, the present invention provides for a compound comprising a carbohydrate moiety and a linker Z, wherein said carbohydrate moiety mimics a glycoepitope recognized by a carbohydrate-binding protein (CBP), wherein said carbohydrate moiety mimics a glycoepitope recognized by a carbohydrate-binding protein (CBP), wherein said CBP is selected from a bacterial exotoxin, an agluttinin and an immune complex deposit-forming immunoglobulin, and wherein said linker Z is —X-A-(B)ₚ-(CH₂)_{q}—Y, wherein X is O or N(R^{a}); R^{a} is H, C₁₋₄alkyl, C₁₋₄alkoxy, CH₂C₆H₅, CH₂CH₂C₆H₅, OCH₂C₆H₅, or OCH₂CH₂C₆H₅; A is C₁₋₇alkylene, OC₁₋₇alkylene, C₁₋₄alkylene-(OCH₂CH₂)ᵣOC₁₋₄alkylene, 0C₁₋₇alkylene-R^{b}, or R^{b}—C₁₋₇alkylene wherein R^{b} is an optionally substituted aryl or an optionally substituted heteroaryl, and wherein r is 0 to 6, preferably r is 1, 2 or 3, and further preferably r is 1; B is NHC(O) or S; p is 0 or 1; q is 0 to 6, preferably q is 0 to 4, and further preferably q is 0, 2 or 3; Y is SH, N₃ or NH₂; and wherein said linker Z is covalently bound via its —X-group to the reducing end of said carbohydrate moiety.

Furthermore, the invention relates to therapeutically acceptable, preferably biodegradable, polymers comprising a multitude of substituents derived from the inventive compounds, wherein said compounds are connected to said polymer backbone by way of the linker Z, and optionally by a spacer, and wherein the connection is effected via the Y-moiety of linker

Z.

Thus, in another aspect, the present invention provides for a polymer comprising a multitude of the inventive compounds, wherein said compounds are connected to the polymer backbone by way of said linker Z, and wherein said connection is effected via the Y-group of said linker Z.

The invention relates also to pharmaceutical compositions comprising the inventive polymers and compounds, respectively, diagnostic kits containing these, and to the use of these compounds for the diagnosis and therapy of bacterial infections, agglutination disorders and immune complex deposit associated disorders.

Thus, in another aspect, the present invention provides for a pharmaceutical composition comprising said inventive polymer or comprising said inventive compound, preferably said inventive compound of formula (I) or of formula (II), or of formula (III), or of formula (IV).

In another aspect, the present invention provides for said inventive compound, preferably said inventive compound of formula (I) or formula (II), or of formula (III), or of formula (IV), or said inventive polymer, preferably comprising said compound, or said inventive pharmaceutical composition for use in a method of treating a (i) bacterial infection wherein preferably said bacterial infection is caused by bacterial exotoxins secreted by Shigella strains, typically and preferably S. dysenteriae, Escherichia coli, Vibrio cholerae, Clostridium difficile, Clostridium botulinum, Clostridium tetani, Bordetella pertussis; (ii) an agglutination disorder, wherein preferably said agglutination disorder is caused by anti-A agglutinins, anti-B agglutinins, anti-I system agglutinins, anti-P system agglutinins, anti-Tn agluttinins, anti-sialyl-Tn agluttinins; and (iii) a disorder caused by immune complex deposit-forming immunoglobulins, wherein preferably said immunoglobulins recognize glycoepitopes on other immunoglobulins or wherein preferably said disorder caused by immune complex deposit-forming immunoglobulins is caused by immunoglobulins binding to the Tn and sialyl-Tn antigen on other immunoglobulins selected from IgG, IgA, IgM.

In another aspect, the present invention provides for said inventive compound, preferably said inventive compound of formula (I) or formula (II), or of formula (III), or of formula (IV), or said inventive polymer, or said inventive pharmaceutical composition for use in a method of diagnosis of a disease associated with CBP-mediated cytotoxicity, agglutination or immune complex deposit formation.

In another aspect, the present invention provides for a diagnostic kit comprising said inventive compound, preferably said inventive compound of formula (I) or formula (II), or of formula (III), or of formula (IV), or said inventive polymer.

In another aspect, the present invention provides for the use of said inventive compound, preferably said inventive compound of formula (I) or formula (II), or of formula (III), or of formula (IV), or said inventive polymer for the diagnosis of a disease associated with CBP-mediated cytotoxicity, agglutination or immune complex deposit formation.

In another aspect, the present invention provides for an use of said inventive compound, preferably said inventive compound of formula (I) or formula (II), or of formula (III), or of formula (IV), or said inventive polymer, for the manufacture of a medicament for the treatment of a (i) bacterial infection wherein preferably said bacterial infection is caused by Shigella strains (e.g. S. dysenteriae), Escherichia coli, Vibrio cholerae, Clostridium difficile, Clostridium botulinum, Clostridium tetani, Bordetella pertussis; (ii) an agglutination disorder, wherein preferably said agglutination disorder is caused by anti-A agglutinins, anti-B agglutinins, anti-I system agglutinins, anti-P system agglutinins, anti-Tn agluttinins, anti-sialyl-Tn agluttinins; and (iii) a disorder caused by immune complex deposit forming immunogloulins wherein preferably said disorder is IgA nephropathy, IgA vasculitis.

In another aspect, the present invention provides for a method of treatment of a disease or disorder associated with CBP-mediated cytotoxicity, agglutination or immune complex deposit formation, wherein said method comprises administering said inventive compound, preferably said inventive compound of formula (I) or formula (II), or of formula (III), or of formula (IV), or of or said inventive polymer in a quantity effective against said disease or disorder, to a warm-blooded animal, preferably to a human, requiring such treatment.

In a further aspect, the present invention provides for a polymer in accordance with the present invention, a compound in accordance with the present invention, or a pharmaceutical composition in accordance with the present invention for use in a method of treating a disease or disorder, wherein said disease or disorder is selected from a bacterial infection, an agglutination disorder or a disorder caused by immune complex deposits, and wherein preferably said disease or disorder is selected from shigellosis, bacillary dysentery, Marlow syndrome, hemolytic-uremic syndrome (HUS), travelers' diarrhea, cholera, Clostridium difficile infection, botulinism, tetanus, pertussis or whooping cough, ABH-incompatible transplantation/transfusion, cold agluttinin disease, paroxysmal cold hemoglobinuria, Tn polyagglutinability syndrome, IgA nephropathy (also known as IgA nephritis or Berger disease or synpharyngitic glomerulonephritis) or IgA vasculitis (also known as Henoch Schönlein Purpura (HSP).

In a further aspect, the present invention provides for a polymer or use in a method of treating a disease or disorder, wherein said disease or disorder is selected from a bacterial infection, an agglutination disorder or a disorder caused by immune complex deposits, preferably a bacterial infection, and wherein preferably said disease or disorder is selected from shigellosis, bacillary dysentery, Marlow syndrome, hemolytic-uremic syndrome (HUS), travelers' diarrhea, cholera, Clostridium difficile infection, botulinism, tetanus, pertussis or whooping cough, ABH-incompatible transplantation/transfusion, cold agluttinin disease, paroxysmal cold hemoglobinuria, Tn polyagglutinability syndrome, IgA nephropathy (also known as IgA nephritis or Berger disease or synpharyngitic glomerulonephritis) or IgA vasculitis (also known as Henoch Schönlein Purpura (HSP); and wherein said polymer comprises a multitude of a compound, wherein said compound comprises a carbohydrate moiety and a linker Z, and wherein said carbohydrate moiety mimics a glycoepitope recognized by a carbohydrate-binding protein (CBP), wherein said CBP is selected from a bacterial exotoxin, an agluttinin and an immune complex deposit-forming immunoglobulin, and wherein said linker Z is —X-A-(B)ₚ-(CH₂)_{q}—Y, wherein
X is O or N(R^{a});
R^{a} is H, C₁₋₄-alkyl, C₁₋₄alkoxy, CH₂C₆H₅, CH₂CH₂C₆H₅, OCH₂C₆H₅, or OCH₂CH₂O₆H₅;
A is C₁₋₇alkylene, OC₁₋₇alkylene, C₁₋₄alkylene-(OCH₂CH₂)ᵣOC₁₋₄alkylene, OC₁₋₇alkylene-R^{b}, or R^{b}—C₁₋₇alkylene wherein R^{b} is an optionally substituted aryl or an optionally substituted heteroaryl, and wherein r is 0 to 6, preferably r is 1, 2 or 3, and further preferably r is 1;
B is NHC(O) or S;
p is 0 or 1;
q is 0 to 6, preferably q is 0 to 4, and further preferably q is 0, 2 or 3;
Y is SH, N₃ or NH₂; and
wherein said linker Z is covalently bound via its —X-group to the reducing end of said carbohydrate moiety; and wherein

said multitude of said compound is connected to the polymer backbone by way of said linker Z, and wherein said connection is effected via the Y-group of said linker Z.

In a further aspect and preferred embodiment of the inventive polymer for use in accordance with the present invention, said compound is a compound of formula (I), formula (II), formula (III) or formula (IV),

wherein formula (I) is

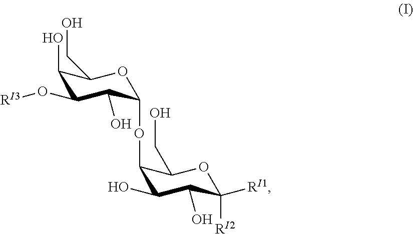

wherein R^{I1} is H or Z or

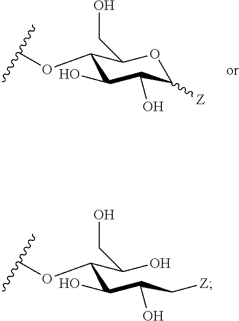

wherein R^{I2} is H or Z;
   wherein, when R^{I1} is H, then R^{I2} is Z; and when R^{I1} is not H, then R^{I2} is H; and thus, R^{I1} and R^{I2} cannot be both H and R^{I1} and R^{I2} cannot be both Z;
and wherein R^{I3} is H or

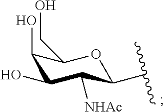

wherein formula (II) is

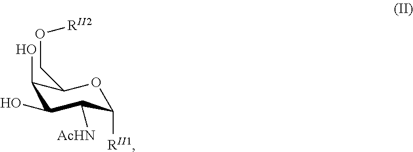

wherein R^{II1} is Z or

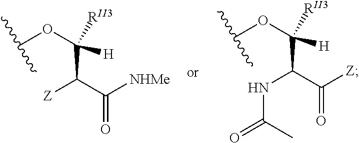

wherein R^{II2} is H or

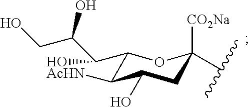

and wherein R^{II3} is H or Me;

wherein formula (III) is

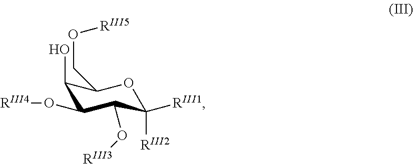

wherein R^{III1} is H or Z or

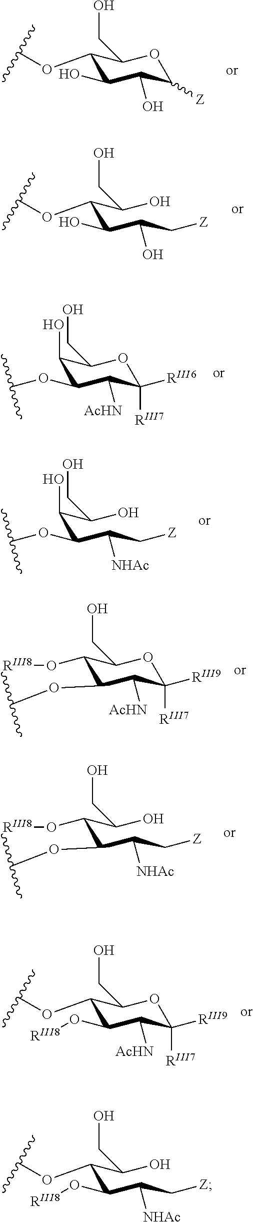

wherein R^{III2} is H or Z;
   wherein when R^{III1} is H, then R^{III2} is Z; and when R^{III1} is not H, then R^{III2} is H;
   and thus, R^{III1} and R^{III2} cannot be both H and R^{III1} and R^{III2} cannot be both Z;
wherein R^{III3} and R^{III8} are independently H or

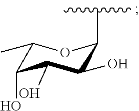

wherein when R^{III1} is not

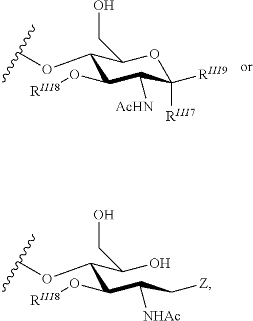

then R^{III3} is H;
wherein R^{III4} is H or

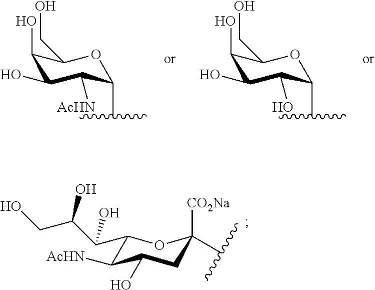

wherein when R^{III4} is not H, then R^{III8} is H;
wherein R^{III3} is H, then R^{III4} is H
wherein R^{III5} is H or

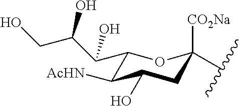

wherein when R^{III4} or R^{III5} is

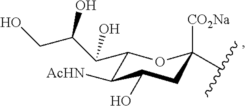

then R^{III1} is H, Z or

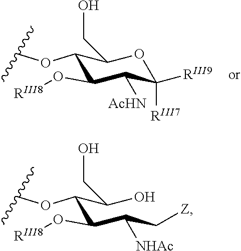

and R^{III3} and R^{III8} are H;
wherein R^{III6} is H or Z or

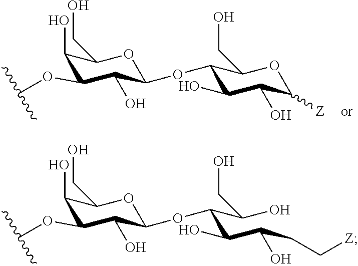

wherein R^{III7} is H or Z;
   wherein when R^{III6} is H, then R^{III7} is Z and when R^{III6} is not H, then R^{III7} is H;
   and thus, R^{III6} and R^{III7} cannot be both H and R^{III6} and R^{III7} cannot be both Z;
wherein R^{III9} is H or Z or

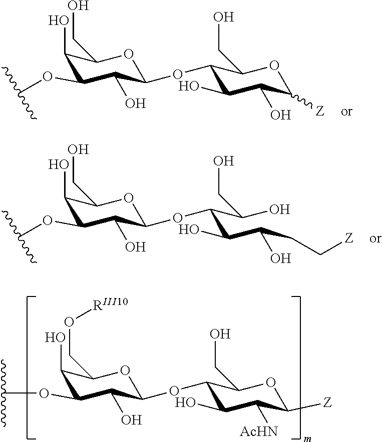

wherein m is 1 to 3;
   wherein when R^{III9} is

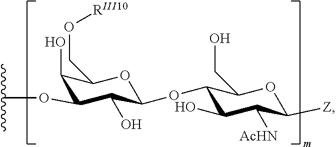

then R^{III3}, R^{III4}, R^{III5} and
R^{III8} are H;
wherein R^{III10} is H or

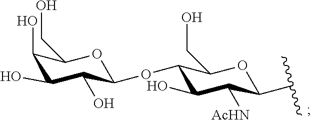

wherein formula (IV) is

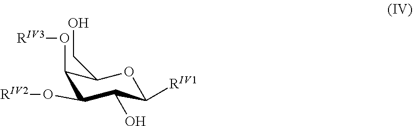

wherein R^{IV1} is

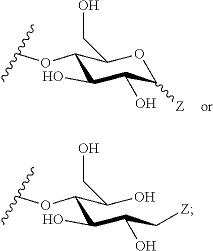

wherein R^{IV2} and R^{IV4} are independently H or

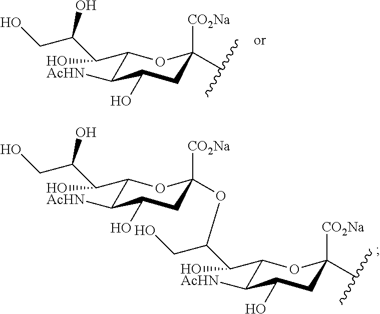

wherein R^{IV3} is H or

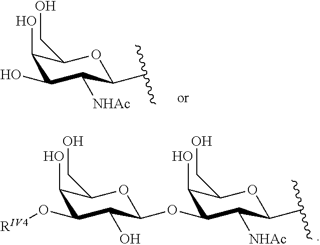

In a further aspect, the present invention provides for a polymer comprising a multitude of a compound, wherein said compound comprises a carbohydrate moiety and a linker Z, and said compound is a compound of formula (I), formula (II), formula (III) or formula (IV), wherein formula (I) is

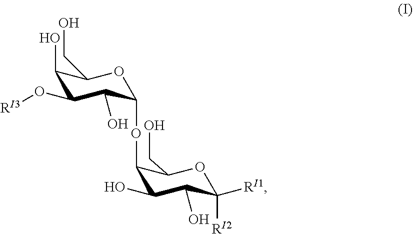

wherein R^{I1} is H or Z or

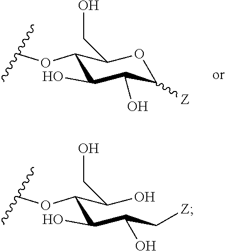

wherein R^{I2} is H or Z;
   wherein, when R^{I1} is H, then R^{I2} is Z; and when R^{I1} is not H, then R^{I2} is H; and
   thus, R^{I1} and R^{I2} cannot be both H and R^{I1} and R^{I2} cannot be both Z;
and wherein R^{I3} is H or

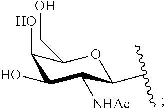

wherein formula (II) is

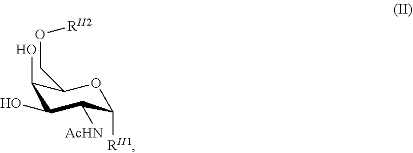

wherein R^{II1} is Z or

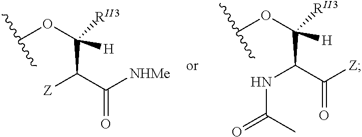

wherein R^{II2} is H or

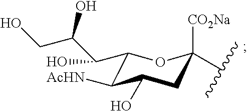

and wherein R^{II3} is H or Me;

wherein formula (III) is

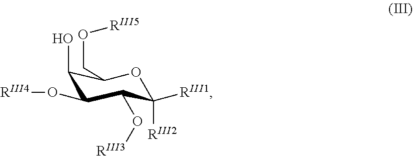

wherein R^{III1} is H or Z or

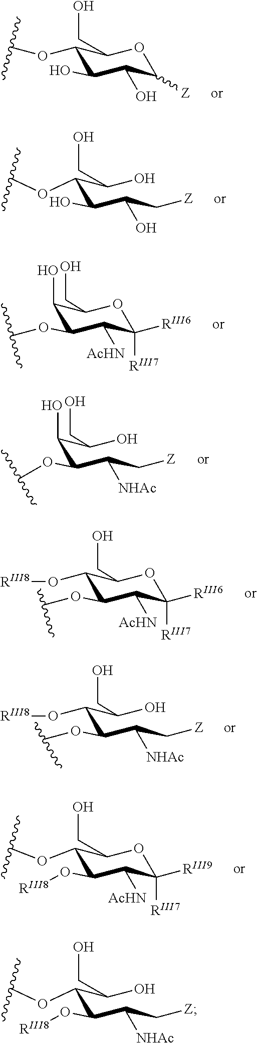

wherein R^{III2} is H or Z;
   wherein when R^{III1} is H, then R^{III2} is Z; and when R^{III1} is not H, then R^{III2} is H;
   and thus, R^{III1} and R^{III2} cannot be both H and R^{III1} and R^{III2} cannot be both Z;
wherein R^{III3} and R^{III8} are independently H or

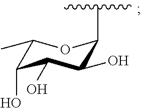

wherein when R^{III1} is not

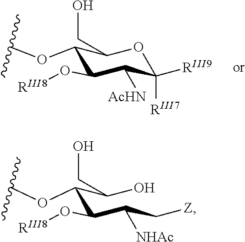

then R^{III3} is H;
wherein R^{III4} is H or

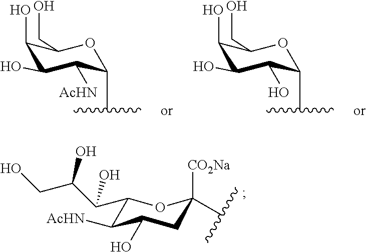

wherein when R^{III4} is not H, then R^{III8} is H;
wherein R^{III3} is H, then R^{III4} is H
wherein R^{III5} is H or

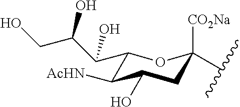

wherein when R^{III4} or R^{III5} is

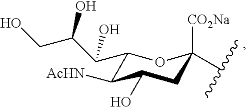

then R^{III1} is H, Z or

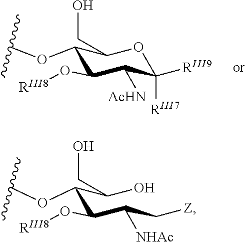

and R^{III3} and R^{III8} are H;
wherein R^{III6} is H or Z or

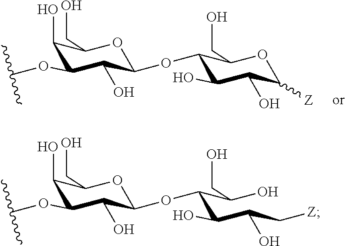

wherein R^{III7} is H or Z;
   wherein when R^{III6} is H, then R^{III7} is Z and when R^{III6} is not H, then R^{III7} is H;
   and thus, R^{III6} and R^{III7} cannot be both H and R^{III6} and R^{III7} cannot be both Z;
wherein R^{III9} is H or Z or

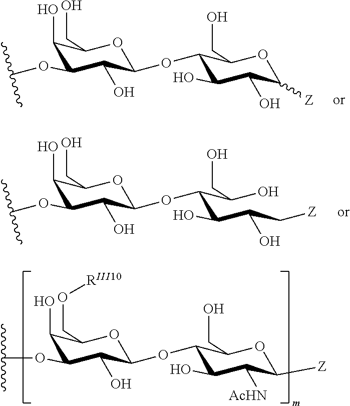

wherein m is 1 to 3;
   wherein when R^{III9} is

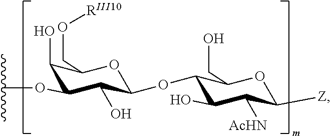

then R^{III3}, R^{III4}, R^{III5} and R^{III8} are H;
wherein R^{III10} is H or

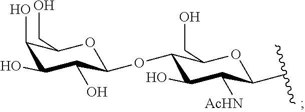

wherein formula (IV) is

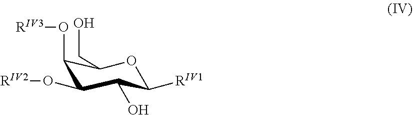

wherein R^{IV1} is

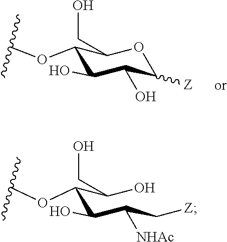

wherein R^{IV2} and R^{IV4} are independently H or

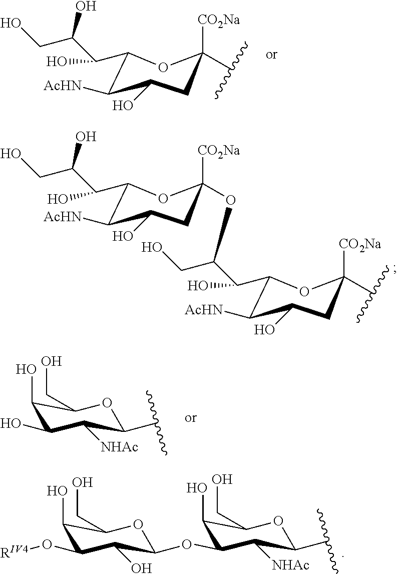

wherein R^{IV3} is H or

Further aspects and embodiments of the present invention will become apparent as this description continues. All embodiments, preferred embodiments and very preferered embodiments described herein apply to any and each aspect of the present invention described herein, even though not always explicitly repeated.

### BRIEF DESCRIPTION OF THE FIGURES

FIGS. 1a,b: Competitive binding assay with an anti-blood group A (BGA) agluttinin. The blood group A antigen (BGA)-coated wells were co-incubated with the carbohydrate polymer 9 (FIG. 1a—0.5 mM highest concentration) or carbohydrate polymer 32 (FIG. 1b—1 mM highest concentration) and anti-BGA agluttinin at a dilution of 1:100. Polymers 9 and 32 are polylysine polymers (average of 400 repeating lysine units) with a defined percentage of lysine residues coupled to blood group A-like carbohydrate antigens. The general abbreviation used is as follows: PL(glycoepitope)ₓ with x defining the percentage of glycoepitope loading in %. In this case the polymers are PL(BGA)₆₈ and PL(BGA)₃₅.

FIG. 2: Competitive binding assay with an anti-blood group B (BGB) agluttinin. The blood group B antigen (BGB)-coated wells were co-incubated with the carbohydrate polymer 35 (1 mM highest concentration) and anti-BGA agluttinin at a dilution of 1:10. Polymer 35 is a polylysine polymer (average of 400 repeating lysine units) with 25% (PL(BGB)₂₅) of lysine residues coupled to the blood group B carbohydrate antigen.

FIG. 3a,b: Competitive binding assay with Shiga like toxin 1 B subunit. The Gb3-coated wells were co-incubated with the carbohydrate polymers 5, 6 (FIG. 3a—Gb3 epitope), and 23 (FIG. 3b—Gb3 epitope mimetic) (500 μM highest concentration) and Shiga like toxin 1 B subunit at a concentration of 2 μg/ml. Polymers 5, 6, and 23 are polylysine polymers (average of 400 repeating lysine units) with 25% (PL(Gb3)₂₅), 40% (PL(Gb3)₄₀) and 42% (PL(Gb3 mimetic)₄₂) of lysine residues coupled to the Gb3 or the Gb3 mimetic carbohydrate epitope.

FIG. 4: Competitive binding assay with cholera toxin B subunit. The GM1 ganglioside-coated wells were co-incubated with the carbohydrate polymer 59 (10 μM highest concentration) and cholera toxin-B subunit-HRP conjugate at a concentration of 0.5 μg/ml. Polymer 59 is a polylysine polymer (average of 400 repeating lysine units) with 28% (PL(GM1)₂₈) of lysine residues coupled to the GM1 carbohydrate epitope.

FIG. 5: Binding assay with anti-Tn IgM. Wells were coated with the carbohydrate polymer 38 (50.0 μg/ml highest concentration) and incubated with anti-Tn IgM at a dilution of 1:700. Polymer 38 is a polylysine polymer (average of 400 repeating lysine units) with 25% (PL(Tn)₂₅) of lysine residues coupled to the Tn epitope.

FIG. 6: Vero cell viability assay with Shiga like toxin 2. Vero cells, which express the Gb3 receptor, were incubated for 48 h with Shiga like toxin 2 alone (concentrations of 0.00001 to 100 μg/ml) or co-incubated with Shiga like toxin 2 and the polymers 5 or 23 at a concentration of 30 μg/ml. Cell viability was measured with the CellTiter Blue® assay. Polymers 5 and 23 are polylysine polymers (average of 400 repeating lysine units) with 25% (PL(Gb3)₂₅) and 42% (PL(Gb3 mimetic)₄₂) of lysine residues coupled to the Gb3 or the Gb3 mimetic carbohydrate epitope.

### DETAILED DESCRIPTION OF THE INVENTION

The invention relates to carbohydrate ligands and moieties, respectively, that mimic glycoepitopes recognized by carbohydrate-binding proteins (CBPs), wherein said CBP is selected from a bacterial exotoxin, an agluttinin and an immune complex deposit-forming immunoglobulin, and particularly glycoepitopes comprised by glycolipids such as the globo- and ganglio-types; red blood cell glycoantigens; and the Tn and sialyl-Tn glycoantigen. The invention further relates to the use of these carbohydrate ligands/moieties, in diagnosis as well as for the treatment of diseases associated with CBP-mediated cytotoxicity, agglutination or immune complex formation. The invention further relates to the use of these carbohydrate ligands/moieties, in diagnosis as well as for the treatment of diseases associated with CBP-mediated cytotoxicity, agglutination or immune complex formation. In particular, the invention relates to compounds of formula (I), (II), (III), (IV), and to therapeutically acceptable polymers comprising a multitude of these compounds, including polymers with loading of compounds of formula (I) or (II) or (III) or (IV).

The compounds of the present invention, and in particular the compounds of the present invention of formula (I), (II), (III) or (IV), recognize CBPs with cytotoxic, agglutinating or immune complex deposit forming properties, in particular glycoepitopes comprised by glycolipids such as the globo- and ganglio-types; red blood cell glycoantigens, and the Tn and sialyl-Tn glycoantigens. The carbohydrate ligands contain linkers that allow coupling to a polymer backbone for multivalent presentation. The glycopolymers resulting from the coupling are superior in the sequestration of CBPs compared to the respective glycan-monomers. The glycopolymers are suitable diagnostic or therapeutic agents to detect and to bind CBPs in particular associated with cytotoxic, agglutinating or immune complex deposit- forming properties.

Thus, in one aspect, the present invention provides for a polymer comprising a multitude of a compound, wherein said compound comprises a carbohydrate moiety and a linker Z, and wherein said carbohydrate moiety mimics a glycoepitope recognized by a carbohydrate-binding protein (CBP), wherein said CBP is selected from a bacterial exotoxin, an agluttinin and an immune complex deposit-forming immunoglobulin, and wherein said linker Z is —X-A-(B)ₚ-(CH₂)_{q}—Y, wherein
X is O or N(R^{a});
R^{a} is H, C₁₋₄-alkyl, C₁₋₄alkoxy, CH₂C₆H₅, CH₂CH₂C₆H₅, OCH₂C₆H₅, or OCH₂CH₂C₆H₅;
A is C₁₋₇alkylene, OC₁₋₇alkylene, C₁₋₄alkylene-(OCH₂CH₂)ᵣOC₁₋₄alkylene, OC₁₋₇alkylene-R^{b}, or R^{b}—C₁₋₇alkylene wherein R^{b} is an optionally substituted aryl or an optionally substituted heteroaryl, and wherein r is 0 to 6, preferably r is 1, 2 or 3, and further preferably r is 1;
B is NHC(O) or S;
p is 0 or 1;
q is 0 to 6, preferably q is 0 to 4, and further preferably q is 0, 2 or 3;
Y is SH, N₃ or NH₂; and

wherein said linker Z is covalently bound via its —X-group to the reducing end of said carbohydrate moiety; and wherein said multitude of said compound is connected to the polymer backbone by way of said linker Z, and wherein said connection is effected via the Y-group of said linker Z; and

wherein said compound is not

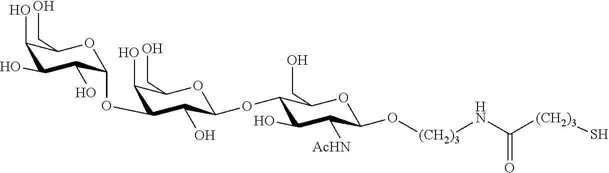

when said polymer backbone is poly-L-lysine; and

wherein said compound is not a compound comprising a carbohydrate moiety and a linker Z, wherein said carbohydrate moiety mimics a glycoepitope comprised by a glycosphingolipid of the nervous system, wherein said linker Z is —N(R^{a})-A-B-CH₂—(CH₂)_{q}—SH, wherein R^{a} is H, C₁₋₄alkyl, C₁-C₄-alkoxy, CH₂C₆H₅, CH₂CH₂C₆H₅, OCH₂C₆H₅, or OCH₂CH₂C₆H₅; A is C₁₋₇alkylene, C₁-C₇-alkoxy, C₁₋₄alkyl—(OCH₂CH₂)ₚO—C₁₋₄alkyl, or C₁-C₇-alkoxy-R^{b}, wherein R^{b} is an optionally substituted aryl or an optionally substituted heteroaryl, and wherein p is 0 to 6, preferably p is 1, 2 or 3, and further preferably p is 1; B is NHC(O), S or CH₂; q is 0 to 6, preferably q is 1, 2, 3 or 4, and further preferably q is 1 or 2; and wherein said linker Z is covalently bound via its —N(R^{a})-group to the reducing end of said carbohydrate moiety.

In a preferred embodiment, said glycoepitope recognized by CBPs is selected from glycolipids such as the globo- and ganglio-types; red blood cell glycoantigens; the Tn and sialyl-Tn glycoantigens. In a further preferred embodiment, said glycoepitopes recognized by CBPs is a globoside, wherein preferably said globoside is selected from Gb3, Gb4. In a further preferred embodiment, said glycoepitopes recognized by CBPs is a red blood cell glycoantigen, wherein preferably said antigen is selected from the A antigen, B antigen, I antigen, P antigen, Lewis^{a} antigen, Lewis^{b} antigen, Lewis^{x} antigen, Lewis^{y} antigen. In a further preferred embodiment, said glycoepitopes recognized by CBPs is the Tn antigen and the sialyl-Tn antigen. In a further preferred embodiment, said glycoepitopes recognized by CBPs is a ganglioside, wherein preferably said ganglioside is selected from GM1a, GM1b, asialo GM1, GD1a, GD1b, GT1a, GT1b, GQ1b, asialo GM2, GM2, GD2, GM3, GD3.

In a preferred embodiment of the present invention, said compound is a compound of formula (I), formula (II), formula (III) or formula (IV),

wherein formula (I) is

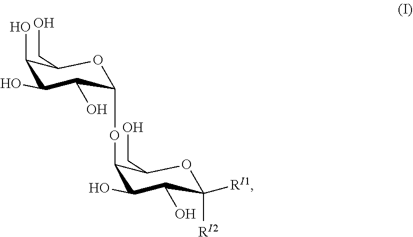

wherein R^{I1} is H or Z or

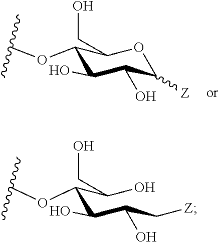

wherein R^{I2} is H or Z;
   wherein, when R^{I1} is H, then R^{I2} is Z; and when R^{I1} is not H, then R^{I2} is H; and
thus, R^{I1} and R^{I2} cannot be both H and R^{I1} and R^{I2} cannot be both Z;

and wherein R^{I3} is H or

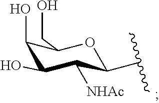

wherein formula (II) is

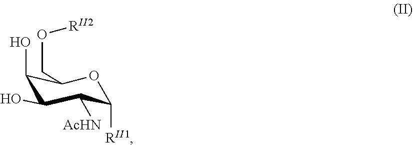

wherein R^{II1} is Z or

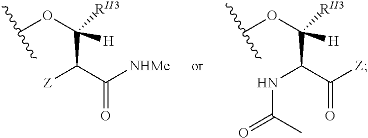

wherein R^{II2} is H or

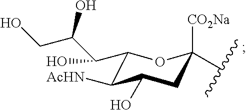

and wherein R^{II3} is H or Me;

wherein formula (III) is

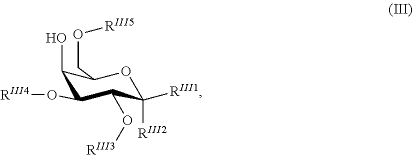

wherein R^{III1} is H or Z or

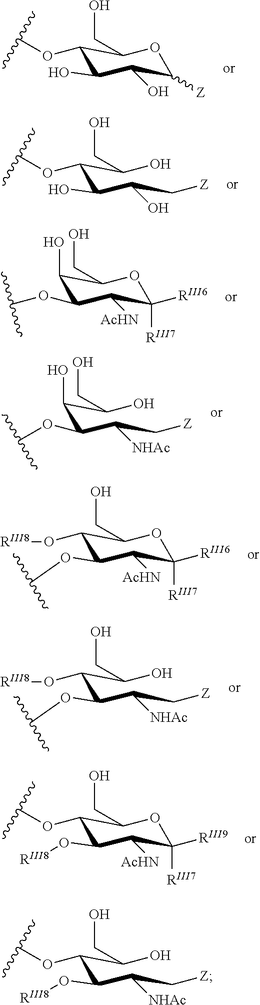

wherein R^{III2} is H or Z;
   wherein when R^{III1} is H, then R^{III2} is Z; and when R^{III1} is not H, then R^{III2} is H;
   and thus, R^{III1} and R^{III2} cannot be both H and R^{III1} and R^{III2} cannot be both Z;
wherein R^{III3} and R^{III8} are independently H or

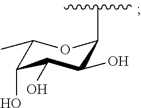

wherein when R^{III1} is not

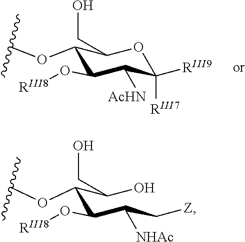

then R^{III3} is H;
wherein R^{III4} is H or

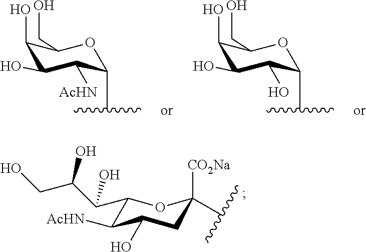

wherein when R^{III4} is not H, then R^{III8} is H;
wherein R^{III3} is H, then R^{III4} is H
wherein R^{III5} is H or

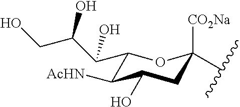

wherein when R^{III4} or R^{III5} is

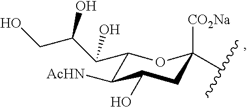

then R^{III1} is H, Z or

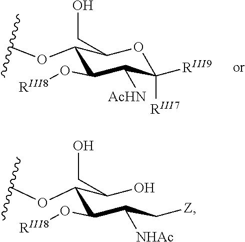

and R^{III3} and R^{III8} are H;
wherein R^{III6} is H or Z or

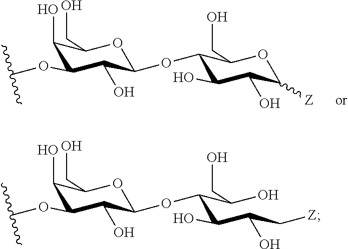

wherein R^{III7} is H or Z;
   wherein when R^{III6} is H, then R^{III7} is Z and when R^{III6} is not H, then R^{III7} is H;
   and thus, R^{III6} and R^{III7} cannot be both H and R^{III6} and R^{III7} cannot be both Z;
wherein R^{III9} is H or Z or

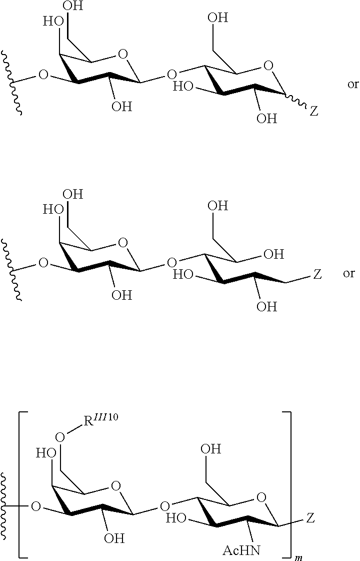

wherein m is 1 to 3;
   wherein when R^{III9} is

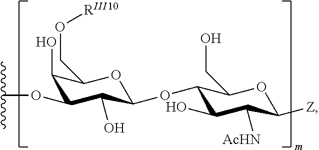

then R^{III3}, R^{II4}, R^{III5} and R^{III8} are H;
wherein R^{III10} is H or

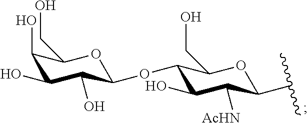

wherein formula (IV) is

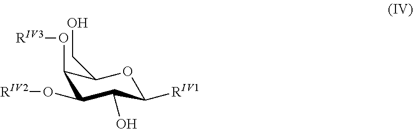

wherein R^{IV1} is

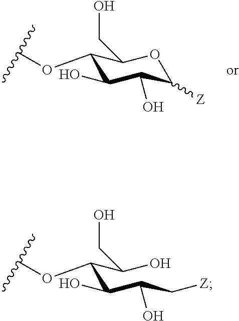

wherein R^{IV2} and R^{IV4} are independently H or

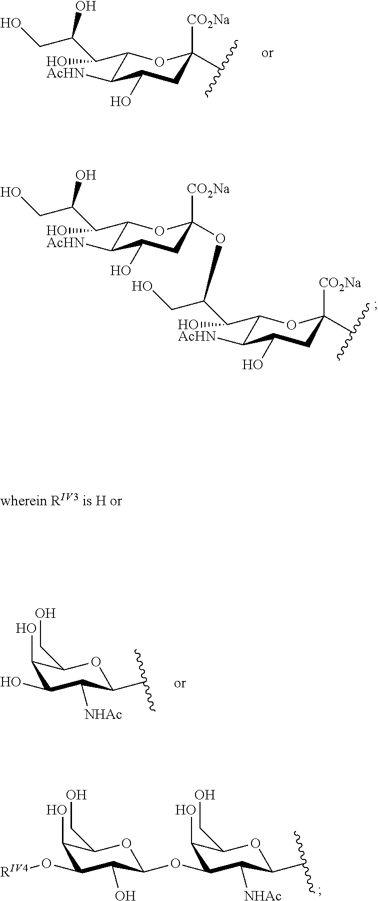

and wherein typically and preferably when said compound is a compound of formula (IV), then said linker Z is not —N(R^{a})-A-B-CH₂—(CH₂)_{q}—SH, wherein R^{a} is H, C₁-C₄-alkyl, C₁-C₄-alkoxy, CH₂C₆H₅, CH₂CH₂C₆H₅, OCH₂C₆H₅, or OCH₂CH₂C₆H₅; A is C₁₋₇alkylene, C₁-C₇-alkoxy, C₁₋₄alkylene-(OCH₂CH₂)ᵣO—C₁₋₄alkylene or C₁-C₇alkoxy-R^{b}, wherein R^{b} is an optionally substituted aryl or an optionally substituted heteroaryl, and wherein r is 0 to 6, preferably r is 1, 2 or 3, and further preferably r is 1; B is NHC(O), S or CH₂; q is 0 to 6, preferably q is 1, 2, 3 or 4, and further preferably q is 1 or 2.

In a further preferred embodiment, said compound is a compound of formula (I), wherein formula (I) is

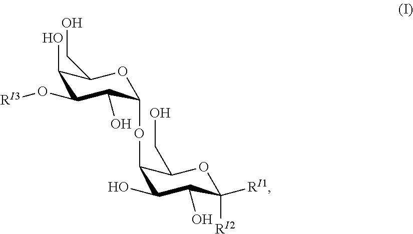

wherein R^{I1} is H or Z or

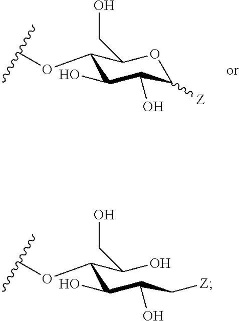

wherein R^{I2} is H or Z;
   wherein, when R^{I1} is H, then R^{I2} is Z; and when R^{I1} is not H, then R^{I2} is H; and thus, R^{I1} and R^{I2} cannot be both H and R^{I1} and R^{I2} cannot be both Z;
and wherein R^{I3} is H or

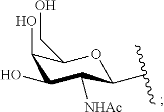

In a further preferred embodiment, said compound is a compound of formula (II), wherein formula (II) is

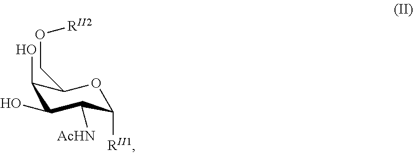

wherein R^{II1} is Z or

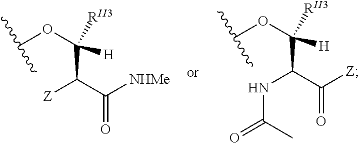

wherein R^{II2} is H or

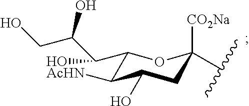

and wherein R^{II3} is H or Me;

In a further preferred embodiment, said compound is a compound of formula (III), wherein formula (III) is

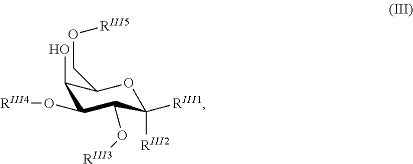

wherein R^{III1} is H or Z or

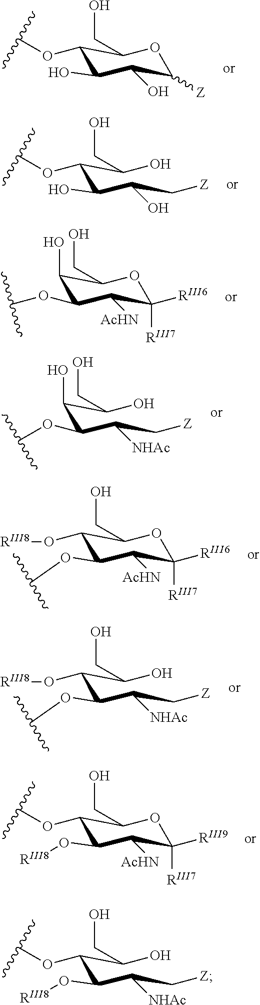

wherein R^{III2} is H or Z;
   wherein when R^{III1} is H, then R^{II2} is Z; and when R^{III1} is not H, then R^{III2} is H;
   and thus, R^{III1} and R^{III2} cannot be both H and R^{III1} and R^{III2} cannot be both Z;
wherein R^{III3} and R^{III8} are independently H or

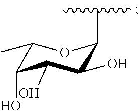

wherein when R^{III1} is not

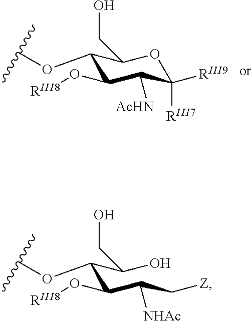

then R^{III3} is H;
wherein R^{III4} is H or

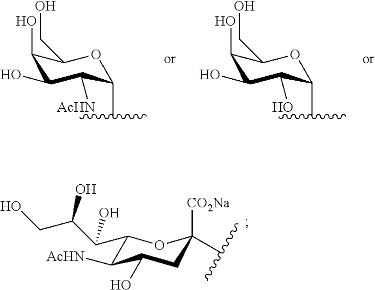

wherein when R^{III4} is not H, then R^{III8} is H;
wherein R^{III3} is H, then R^{III4} is H
wherein R^{III5} is H or

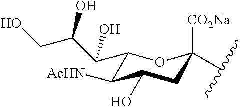

wherein when R^{III4} or R^{III5} is

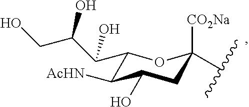

then R^{III1} is H, Z or

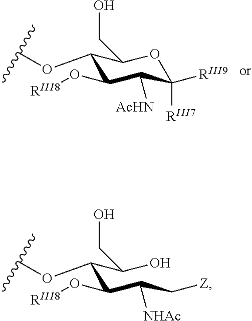

and R^{III3} and R^{III8} are H;
wherein R^{III6} is H or Z or

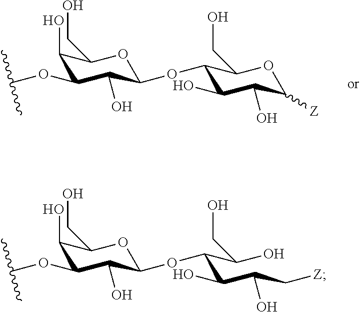

wherein R^{III7} is H or Z;
   wherein when R^{III6} is H, then R^{III7} is Z and when R^{III6} is not H, then R^{III7} is H;
   and thus, R^{III6} and R^{III7} cannot be both H and R^{III6} and R^{III7} cannot be both Z;
wherein R^{III9} is H or Z or

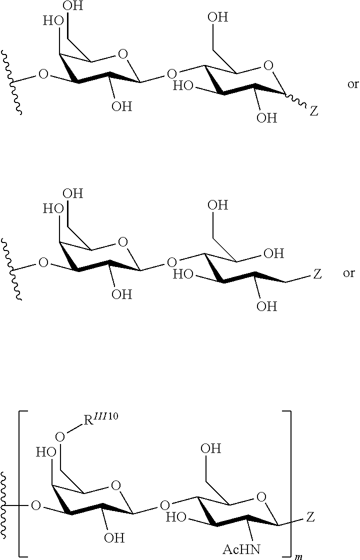

wherein m is 1 to 3;
   wherein when R^{III9} is

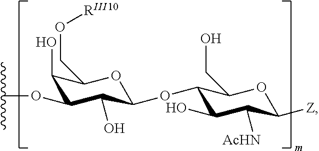

then R^{III3}, R^{III4}, R^{III5} and
R^{III8} are H;
wherein R^{III10} is H or

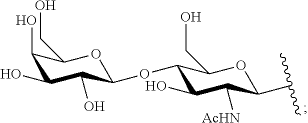

In a further preferred embodiment, said compound is a compound of formula (III), wherein formula (III) is

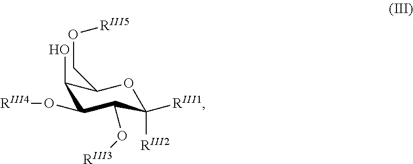

wherein R^{III1} is H or Z or

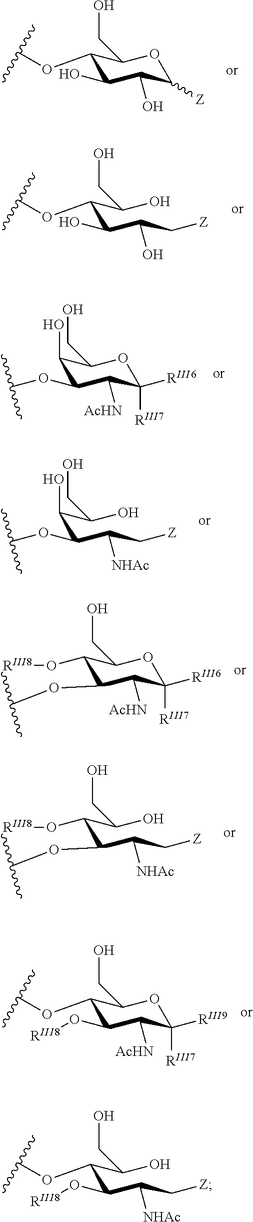

wherein R^{III2} is H or Z;
   wherein when R^{III1} is H, then R^{III2} is Z; and when R^{III1} is not H, then R^{III2} is H;
   and thus, R^{III1} and R^{III2} cannot be both H and R^{III1} and R^{III2} cannot be both Z;
wherein R^{III3} and R^{III8} are independently H or

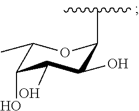

wherein when R^{III1} is not

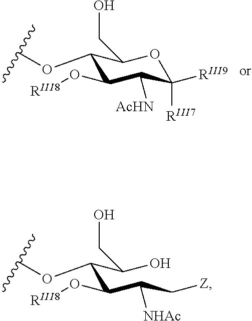

then R^{III3} is H;
wherein R^{III4} is H or

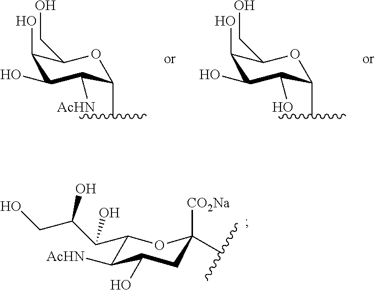

wherein when R^{III4} is not H, then R^{III8} is H;
wherein R^{III3} is H, then R^{III4} is H
wherein R^{III5} is H or

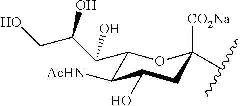

wherein when R^{III4} or R^{III5} is

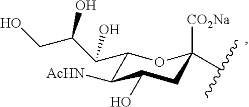

then R^{III1} is H, Z or

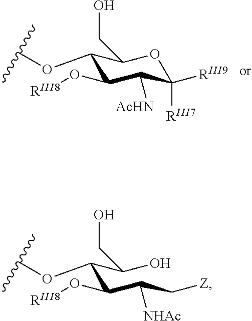

and R^{III3} and R^{III8} are H;
wherein R^{III6} is H or Z or

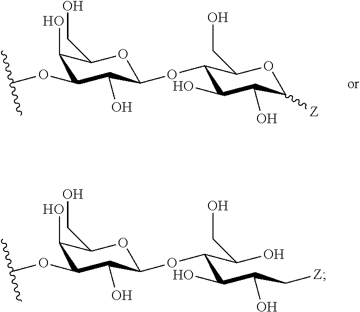

wherein R^{III7} is H or Z;
   wherein when R^{III6} is H, then R^{III7} is Z and when R^{III6} is not H, then R^{III7} is H;
   and thus, R^{III6} and R^{III7} cannot be both H and R^{III6} and R^{III7} cannot be both Z;
wherein R^{III9} is H or Z or

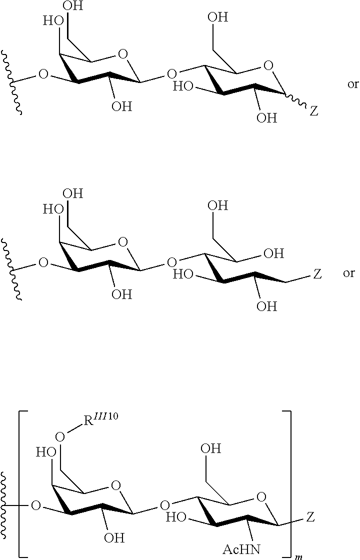

wherein m is 1 to 3;
   wherein when R^{III9} is

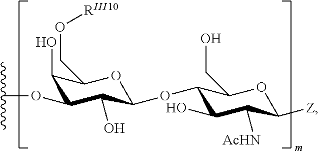

then R^{III3}, R^{III4}, R^{III5} and
R^{III8} are H;
wherein R^{III10} is H or

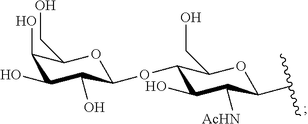

In a further preferred embodiment, said compound is a compound of formula (IV), wherein formula (IV) is

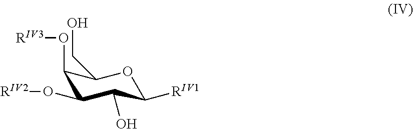

wherein R^{IV1} is

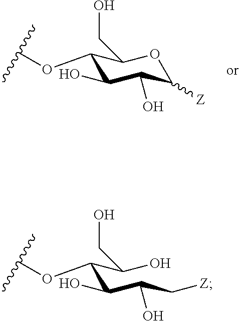

wherein R^{IV2} and R^{IV4} are independently H or

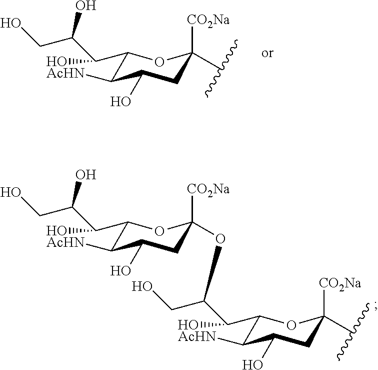

wherein R^{IV3} is H or

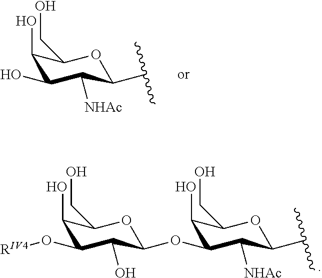

In a further preferred embodiment, said compound is a compound of formula (IV), wherein formula (IV) is

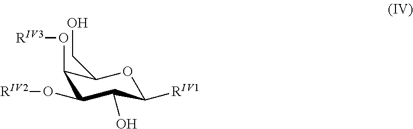

wherein R^{IV1} is

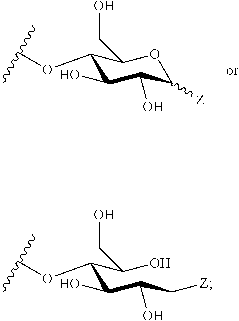

wherein R^{IV2} is

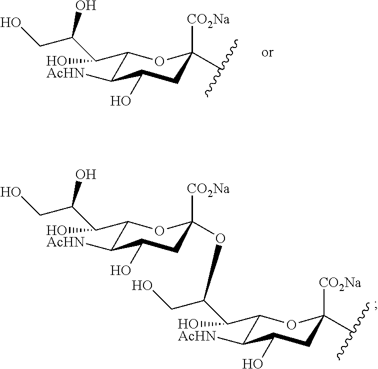

wherein R^{IV3} is

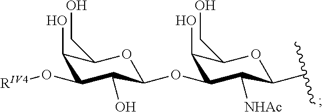

wherein R^{IV4} is H or

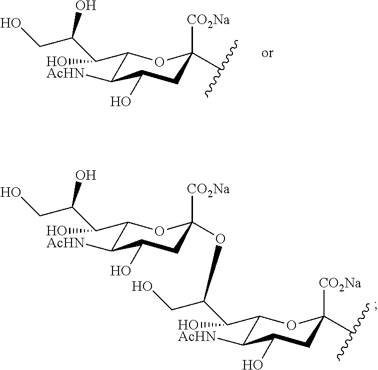

In a further preferred embodiment, said compound is a compound of formula (IV), wherein formula (IV) is

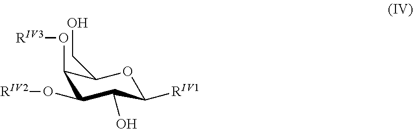

wherein R^{IV1} is

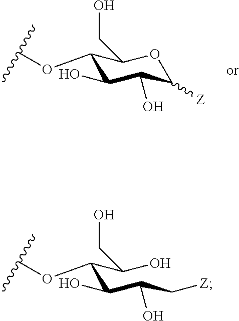

wherein R^{IV2} is

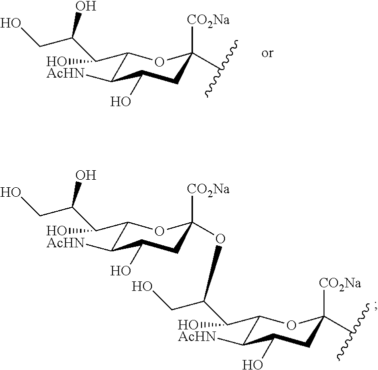

wherein R^{IV3} is

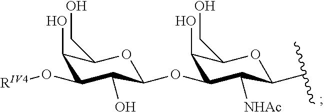

wherein R^{IV4} is H

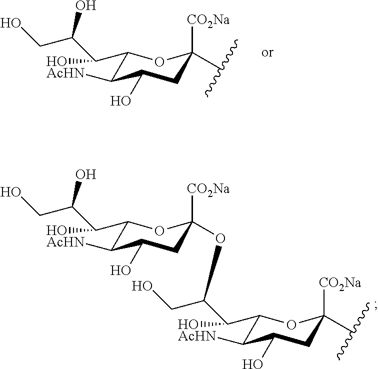

wherein when R^{IV2} is αNeu5Ac, then R^{III4} is H; and when R^{IV2} is Neu5Ac(α2-8α)Neu5Ac, then R^{IV4} is αNeu5Ac or Neu5Ac(α2-8α)Neu5Ac;

In a further preferred embodiment, said bacterial exotoxin is an AB Toxin, wherein preferably said AB toxin is a binary AB toxin or a heterohexameric AB₅ toxin. Preferably said binary AB toxin is tetanus toxin, botulinum toxin or toxin A. Preferably said heterohexameric AB₅ toxin is Shiga toxin, Shiga-like toxin/vero toxin, cholera toxin, heat-labile enterotoxin or pertussis toxin. In a further preferred embodiment, said bacterial exotoxin is selected from tetanus toxin, botulinum toxin, toxin A, Shiga toxin, Shiga-like toxin/vero toxin, cholera toxin, heat-labile enterotoxin or pertussis toxin, wherein further preferably said bacterial exotoxin is Shiga toxin or Shiga-like toxin/vero toxin.

In a further preferred embodiment, said agluttinin is a red blood cell-agglutinating immunoglobulin which preferably bind to glycoepitopes of the ABH system, and wherein preferably said red blood cell-agglutinating immunoglobulin recognizes the glycoantigens of the ABH system, I/i system or/and the P system. In a further preferred embodiment, said agluttinin recognizes the A, B, H, I or P gycoepitopes.

In a further preferred embodiment, said immune complex deposit-forming immunoglobulin is an immunoglobulin which recognize one or more glycoepitopes on other immunoglobulins, and wherein preferably said immune complex deposit-forming immunoglobulin is an immunoglobulin which recognize one or more glycoepitopes on IgG, IgA and IgM.

In a preferred embodiment, said glycoepitopes recognized by CBPs is selected from glycolipids such as the globo- and ganglio-types; red blood cell glycoantigens; the Tn and sialyl-Tn glycoantigens. In a further preferred embodiment, said glycoepitopes recognized by CBPs is a globoside, wherein preferably said globoside is selected from Gb3, Gb4. In a further preferred embodiment, said glycoepitopes recognized by CBPs is a red blood cell glycoantigen, wherein preferably said antigen is selected from the A antigen, B antigen, I antigen, P antigen, Lewis^{a} antigen, Lewis^{b} antigen, Lewis^{x} antigen, Lewis^{y} antigen. In a further preferred embodiment, said glycoepitopes recognized by CBPs is the Tn antigen and the sialyl-Tn antigen. In a further preferred embodiment, said glycoepitopes recognized by CBPs is a ganglioside, wherein preferably said ganglioside is selected from GM1a, GM1b, asialo GM1, GD1a, GD1b, GT1a, GT1b, GQ1b, asialo GM2, GM2, GD2, GM3, GD3.

The scope of the present invention comprises carbohydrate moieties mimicking glycoepitopes comprised by the sialyl-Tn antigen and gangliosides. Preferred compounds mimicking glycoepitopes comprised by gangliosides in accordance with the present invention are compounds of the formula (II), (III) and (IV) as defined herein, wherein at least one of sialic acid moiety is replaced by a replacement moiety as shown and defined in formula (IIa) or formula (IIb)

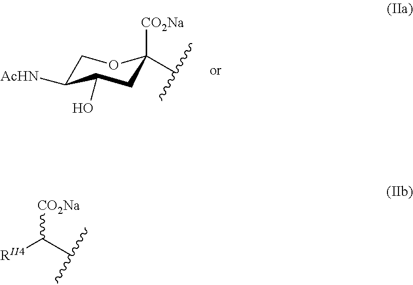

wherein for said replacement moiety of formula (IIb), R^{II4} is H, C₁₋₈alkyl, C₁₋₈alkenyl, C₁₋₈alkynyl, aryl, substituted aryl, wherein preferably said substitution of said aryl is by halogen, C₁₋₈alkoxy, C₁₋₈alkyl; heteroaryl, substituted heteroaryl, wherein preferably said substitution of said hetereoaryl is by halogen, C₁₋₈alkoxy, C₁₋₈alkyl; arylalkyl, substituted arylalkyl, wherein preferably said substitution of said arylalkyl is by halogen, C₁₋₈alkoxy, C₁₋₈alkyl; heteroarylalkyl, substituted heteroarylalkyl, wherein preferably said substitution of said heteroarylalkyl is by halogen, C₁₋₈alkoxy, C₁₋₈alkyl; cycloalkyl, t-butyl, adamantyl, triazolyl all of which independently substituted with C₁₋₈alkyl, aryl, heteroaryl, halogen.

In another preferred embodiment, said compound is a compound of any one of formula 3*, 8*, 22*, 26*, 31*, 34*, 37*, 45*, 47*-58* as depicted below.

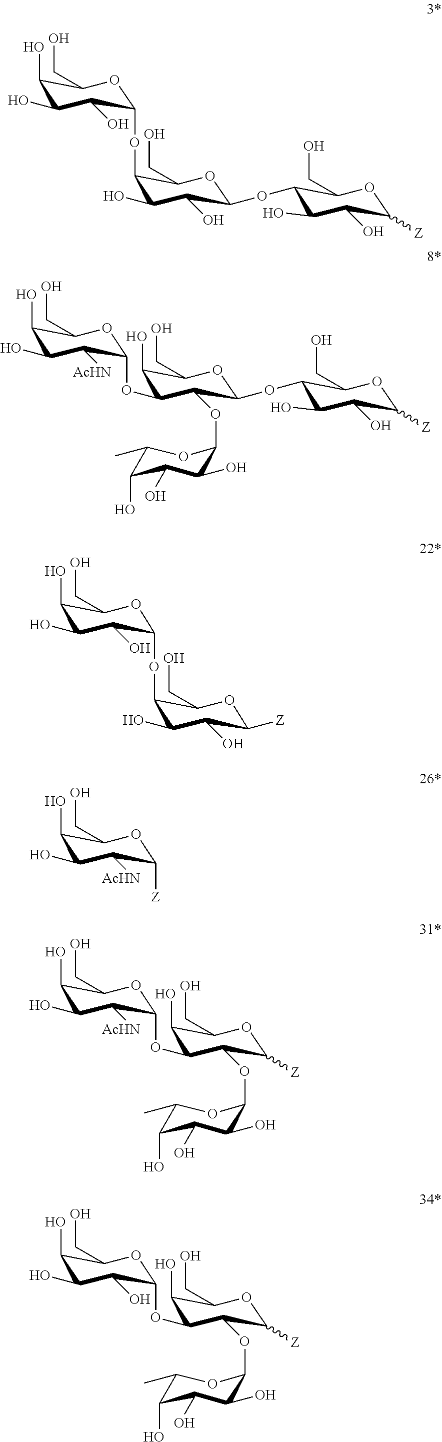

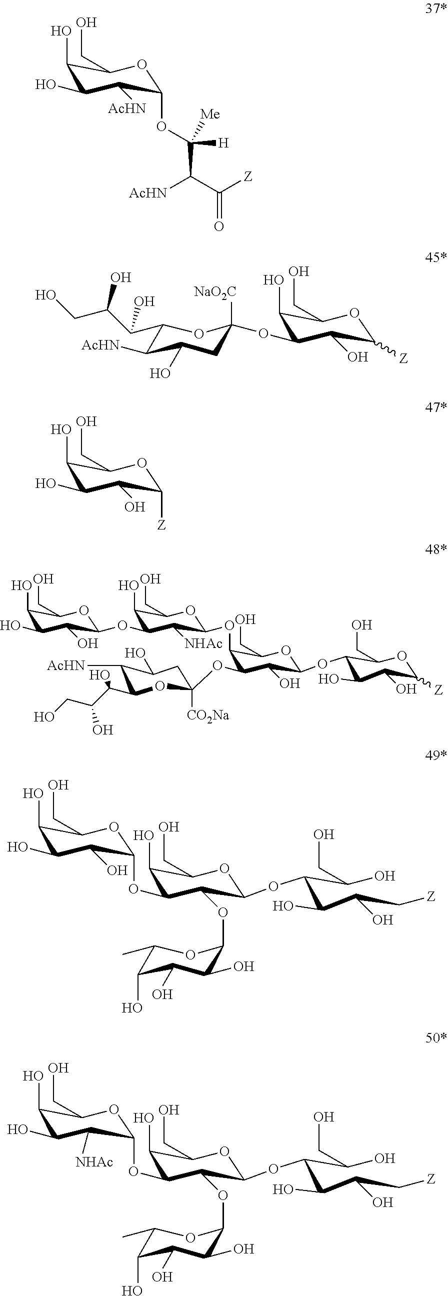

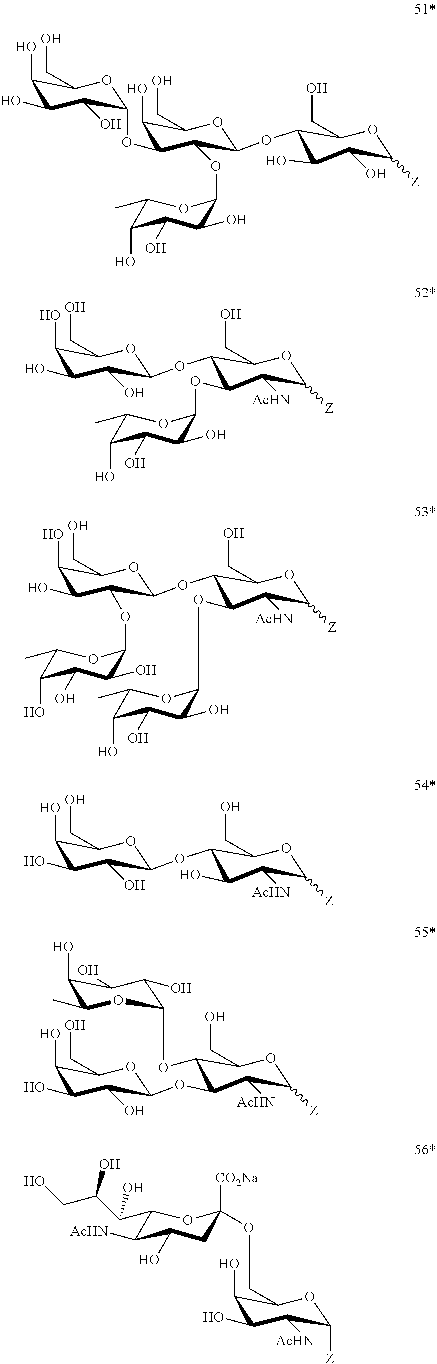

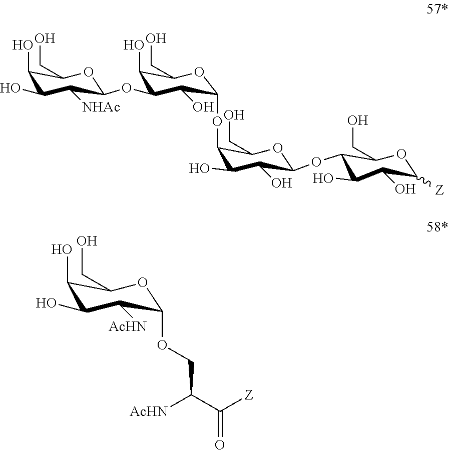

said linker Z is —X-A-(B)ₚ-(CH₂)_{q}—Y, wherein
X is O or N(R^{a});
R^{a} is H, C₁₋₄alkyl, C₁₋₄alkoxy, CH₂C₆H₅, CH₂CH₂C₆H₅, OCH₂C₆H₅, or OCH₂CH₂C₆H₅;
A is C₁₋₇alkylene, OC₁₋₇alkylene, C₁₋₄alkylene-(OCH₂CH₂)ᵣOC₁₋₄alkylene, OC₁₋₇alkylene-R^{b}, or R^{b}-C₁₋₇alkylene wherein R^{b} is an optionally substituted aryl or an optionally substituted heteroaryl, and wherein r is 0 to 6, preferably r is 1, 2 or 3, and further preferably r is 1;
B is NHC(O) or S;
p is 0 or 1;
q is 0 to 6, preferably q is 0 to 4, and further preferably q is 0, 2 or 3;
Y is SH, N₃ or NH₂; and

wherein said linker Z is covalently bound via its —X-group to the reducing end of said carbohydrate moiety; and wherein

said multitude of said compound is connected to the polymer backbone by way of said linker Z, and wherein said connection is effected via the Y-group of said linker Z.

In a further preferred embodiment, said compound is a compound of formula 3*, 22* or 57*. In a further preferred embodiment, said compound is a compound of formula 26*, 37*, 49* or 58*. In a further preferred embodiment, said compound is a compound of formula 26*, 37*, 56* or 58*. In a further preferred embodiment, said compound is a compound of any one of formula 8*, 31*, 34*, 37*, 45*, 47*, 50*-56*, 58*. In a further preferred embodiment, said compound is a compound of any one of formula 8*, 31*, 34*, 45*, 47*, 49*-55*. In a further preferred embodiment, said compound is a compound of any one of formula 3*, 8*, 22*. 26*, 31*, 34*, 37* and 48*. In a further preferred embodiment, said compound is a compound of formula 48*.

In a further preferred embodiment, said compound is a compound of formula 45*, 49*, 48* or 56* wherein at least one of sialic acid moiety is replaced by a replacement moiety as shown and defined in formula (IIa) or formula (IIb)

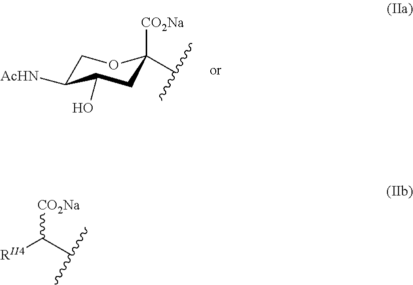

wherein for said replacement moiety of formula (IIb), R^{II4} is H, C₁₋₈alkyl, C₁₋₈alkenyl, C₁₋₈alkynyl, aryl, substituted aryl, wherein preferably said substitution of said aryl is by halogen, C₁₋₈alkoxy, C₁₋₈alkyl; heteroaryl, substituted heteroaryl, wherein preferably said substitution of said hetereoaryl is by halogen, C₁₋₈alkoxy, C₁₋₈alkyl; arylalkyl, substituted arylalkyl, wherein preferably said substitution of said arylalkyl is by halogen, C₁₋₈alkoxy, C₁₋₈alkyl; heteroarylalkyl, substituted heteroarylalkyl, wherein preferably said substitution of said heteroarylalkyl is by halogen, C₁₋₈alkoxy, C₁₋₈alkyl; cycloalkyl, t-butyl, adamantyl, triazolyl all of which independently substituted with C₁₋₈alkyl, aryl, heteroaryl, halogen.

In a preferred embodiment of said linker Z, X is N(R^{a}); R^{a} is H, C₁₋₄alkyl, C₁₋₄alkoxy, CH₂C₆H₅, CH₂CH₂C₆H₅, OCH₂C₆H₅, or OCH₂CH₂C₆H₅; A is C₁₋₇alkylene, OC₁₋₇alkylene, C₁₋₄alkylene-(OCH₂CH₂)ᵣO—C₁-C₄-alkylene, OC₁₋₇alkylene-R^{b} or R^{b}—C₁₋₇alkylene wherein R^{b} is an optionally substituted aryl or an optionally substituted heteroaryl, and wherein r is 0 to 6, preferably r is 1, 2 or 3, and further preferably r is 1; B is NHC(O) or S; p is 0 or 1; q is 0 to 6, preferably q is 0 to 4, and further preferably q is 0, 2 or 3; Y is SH, N₃ or NH₂.

In a further preferred embodiment, said X is N(R^{a}), and said R^{a} is H, CH₃, CH₂CH₃, CH₂CH₂CH₃, CH(CH₃)₂, OCH₃, OCH₂CH₃, OCH₂CH₂CH₃, CH₂C₆H₅, OCH₂C₆H₅; and said A is O(CH₂)ᵣ, (CH₂)ᵣ, CH₂CH₂(OCH₂CH₂)ᵣ, (OCH₂CH₂)ᵣ, O(CH₂)ᵣC₆H₅, C₆H₅(CH₂)ᵣ.

In a further preferred embodiment, said X is N(R^{a}), said R^{a} is CH₃ or OCH₃; said A is O(CH₂)ᵣ, (CH₂)ᵣ, CH₂(OCH₂CH₂)ᵣOCH₂, (OCH₂CH₂)ᵣOCH₂CH₂ or O(CH₂)ᵣC₆H₅; and said B is NHC(O).

In a further preferred embodiment, said X is N(R^{a}), said R^{a} is CH₃; A is O(CH₂)ᵣ, (OCH₂CH₂)ᵣOCH₂CH₂ or O(CH₂)ᵣC₆H₅; and B is NHC(O) or S. Preferably, when B is S and A is (CH₂)ᵣCH₂, then q is 1 to 5, preferably 1, 2 or 3.

In a further preferred embodiment, said X is N(R^{a}), said R^{a} is CH₃ or OCH₃; A is O(CH₂)ᵣ, (CH₂)ᵣ, CH₂(OCH₂CH₂)ᵣOCH₂, (OCH₂CH₂)ᵣOCH₂CH₂ or O(CH₂)ᵣC₆H₅; B is NHC(O) or S; and q is 1 to 5, preferably 1, 2 or 3, preferably 2 or 3.

In another embodiment, said X is N(R^{a}); R^{a} is H, CH₃, CH₂CH₃, CH₂CH₂CH₃, CH(CH₃)₂, OCH₃, OCH₂CH₃, OCH₂CH₂CH₃, CH₂C₆H₅, OCH₂C₆H₅; A is O(CH₂)ᵣ, (CH₂)ᵣ, CH₂CH₂(OCH₂CH₂)ᵣ, (OCH₂CH₂)ᵣ, O(CH₂)ᵣC₆H₅, C₆H₅(CH₂)ᵣ; B is NHC(O) or S; p is 0 or 1; q is 0 to 6, preferably q is 0 to 4, and further preferably q is 0, 2 or 3; Y is SH, N₃ or NH₂.

In another preferred embodiment, said X is N(R^{a}); and said R^{a} is CH₃ or OCH₃; A is O(CH₂)ᵣ, (CH₂)ᵣ, CH₂(OCH₂CH₂)ᵣOCH₂, (OCH₂CH₂)ᵣOCH₂CH₂ or O(CH₂)ᵣC₆H₅; and B is NHC(O) or S. Preferably, when B is S, and A is (CH₂)ᵣ, then q is 1 to 5, preferably 1, 2 or 3.

In a further preferred embodiment of said linker Z, said X is O; A is C₁₋₇alkylene, C₁₋₄alkylene-(OCH₂CH₂)ᵣOC₁₋₄alkylene or R^{b}—C₁₋₇alkylene, wherein R^{b} is an optionally substituted aryl or an optionally substituted heteroaryl, and wherein r is 0 to 6, preferably r is 1, 2 or 3, and further preferably r is 1; B is NHC(O) or S; p is 0 or 1, and wherein preferably p is 1; q is 0 to 6, preferably q is 0 to 4, and further preferably q is 0, 2 or 3; Y is SH, N₃ or NH₂.

In a further preferred embodiment, said X is O, A is C₁₋₇alkylene, C₁₋₄alkylene-(OCH₂CH₂)ᵣOC₁₋₄alkylene or R^{b}—C₁₋₇alkylene, wherein R^{b} is an optionally substituted aryl or an optionally substituted heteroaryl, and wherein r is 0 to 6, preferably r is 1, 2 or 3, and further preferably r is 1; B is NHC(O) or S; p is 1, q is 0 to 6, preferably q is 1 to 4, and further preferably q is 1, 2 or 3.

In a further preferred embodiment, said X is O; A is R^{b}—C₁₋₇alkylene wherein R^{b} is an optionally substituted aryl or an optionally substituted heteroaryl, and wherein r is 0 to 6, preferably r is 1, 2 or 3, and further preferably r is 1; B is NHC(O) or S; p is 0 or 1; q is 0 to 6, preferably q is 0 to 4, and further preferably q is 0, 2 or 3; Y is SH, N₃ or NH₂.

In a further preferred embodiment, said X is O; A is R^{b}—C₁₋₇alkylene wherein R^{b} is an optionally substituted aryl or an optionally substituted heteroaryl, and wherein r is 0 to 6, preferably r is 1, 2 or 3, and further preferably r is 1; B is NHC(O) or S; p is1; q is 1 to 6, preferably q is 1, 2, 3 or 4, and further preferably q is 1, 2 or 3; Y is SH, N₃ or NH₂.

In a further preferred embodiment of said linker Z, said X is O; □A is (CH₂)ᵣ, CH₂CH₂(OCH₂CH₂)ᵣ, C₆H₅(CH₂)ᵣ; B is NHC(O) or S; p is 0 or 1; q is 0 to 6, preferably q is 0 to 4, and further preferably q is 0, 2 or 3; Y is SH, N₃ or NH₂.

In a further preferred embodiment, said compound is a compound of formula (I), formula (II) or formula (III), and wherein said linker Z is —X-A-(B)ₚ-(CH₂)_{q}—Y, wherein X is N(R^{a}); R^{a} is H, C₁₋₄-alkyl, C₁₋₄-alkoxy, CH₂C₆H₅, CH₂CH₂C₆H₅, OCH₂C₆H₅, or OCH₂CH₂C₆H₅; A is C₁₋₇alkylene, OC₁₋₇alkylene C₁₋₄alkylene-(OCH₂CH₂)ᵣOC₁₋₄alkylene, OC₁₋₇alkylene-R^{b}, or R^{b}-C₁₋₇alkylene wherein R^{b} is an optionally substituted aryl or an optionally substituted heteroaryl, and wherein r is 0 to 6, preferably r is 1, 2 or 3, and further preferably r is 1; B is NHC(O) or S; p is 0 or 1; q is 0 to 6, preferably q is 0 to 4, and further preferably q is 0, 2 or 3; Y is SH, N₃ or NH₂.

In a further preferred embodiment, said compound is a compound of formula (I), formula (II) or formula (III), and wherein said linker Z is —X-A-(B)ₚ-(CH₂)_{q}—Y, wherein X is N(R^{a}); R^{a} is H, C₁₋₄-alkyl, C₁₋₄-alkoxy, CH₂C₆H₅, CH₂CH₂C₆H₅, OCH₂C₆H₅, or OCH₂CH₂C₆H₅; A is C₁₋₇alkylene, OC₁₋₇alkylene C₁₋₄alkylene-(OCH₂CH₂)ᵣOC₁₋₄alkylene, OC₁₋₇alkylene-R^{b}, or R^{b}—C₁₋₇alkylene wherein R^{b} is an optionally substituted aryl or an optionally substituted heteroaryl, and wherein r is 0 to 6, preferably r is 1, 2 or 3, and further preferably r is 1; B is NHC(O) or S; p is 0 or 1; q is 0 to 6, preferably q is 0 to 4, and further preferably q is 0, 2 or 3; Y is SH, N₃ or NH₂. Further preferably, said compound is compound of formula 3*, 22* or 58*. Further preferably, said compound is compound of formula 3* or a compound of formula 22*.

In a further preferred embodiment, said compound is a compound of formula (II), and wherein said linker Z is —X-A-(B)ₚ-(CH₂)_{q}—Y, wherein X is N(R^{a}); R^{a} is H, C₁₋₄-alkyl, C₁₋₄-alkoxy, CH₂C₆H₅, CH₂CH₂C₆H₅, OCH₂C₆H₅, or OCH₂CH₂C₆H₅; A is C₁₋₇alkylene, OC₁₋₇alkylene C₁₋₄alkylene-(OCH₂CH₂)ᵣOC₁₋₄alkylene, OC₁₋₇alkylene-R^{b}, or R^{b}—C₁₋₇alkylene wherein R^{b} is an optionally substituted aryl or an optionally substituted heteroaryl, and wherein r is 0 to 6, preferably r is 1, 2 or 3, and further preferably r is 1; B is NHC(O) or S; p is 0 or 1; q is 0 to 6, preferably q is 0 to 4, and further preferably q is 0, 2 or 3; Y is SH, N₃ or NH₂. Further preferably, said compound is compound of formula 26*, 37*, 49*, 58*. Further preferably, said compound is compound of formula 26*, 37*, 56*, 58*. Again further preferably, said compound is a compound of formula 26* or a compound of formula 37*.

In a further preferred embodiment, said compound is a compound of formula (I), formula (II), formula (III) or fomula (IV), and wherein said linker Z is —X-A-(B)ₚ-(CH₂)_{q}—Y, wherein X is O; A is C₁₋₇alkylene, C₁₋₄alkylene-(OCH₂CH₂)ᵣOC₁₋₄alkylene or R^{b}—C₁₋₇alkylene, wherein R^{b} is an optionally substituted aryl or an optionally substituted heteroaryl, and wherein r is 0 to 6, preferably r is 1, 2 or 3, and further preferably r is 1; B is NHC(O) or S; p is 0 or 1; q is 0 to 6, preferably q is 0 to 4, and further preferably q is 0, 2 or 3; Y is SH, N₃ or NH₂.

In a further preferred embodiment, said compound is a compound of formula (I), formula (II), formula (III) or formula (IV), and wherein said linker Z is —X-A-(B)ₚ-(CH₂)_{q}—Y, wherein X is O; A is C₁₋₇alkylene, C₁₋₄alkylene-(OCH₂CH₂)ᵣOC₁₋₄alkylene or R^{b}—C₁₋₇alkylene, wherein R^{b} is an optionally substituted aryl or an optionally substituted heteroaryl, and wherein r is 0 to 6, preferably r is 1, 2 or 3, and further preferably r is 1; B is NHC(O) or S; p is 1; q is 1 to 6, preferably q is 1, 2, 3 or 4, and further preferably q is 1, 2 or 3; Y is SH, N₃ or NH₂.

In a further preferred embodiment, said compound is a compound of formula (I), formula (II), formula (III) or fomula (IV), and wherein said linker Z is —X-A-(B)ₚ-(CH₂)_{q}—Y, wherein X is O; A is R^{b}—C₁₋₇alkylene, wherein R^{b} is an optionally substituted aryl or an optionally substituted heteroaryl, and wherein r is 0 to 6, preferably r is 1, 2 or 3, and further preferably r is 1; B is NHC(O) or S; p is 0 or 1; q is 0 to 6, preferably q is 0 to 4, and further preferably q is 0, 2 or 3; Y is SH, N₃ or NH₂.

In a further preferred embodiment, said compound is a compound of formula (I), formula (II), formula (III) or fomula (IV), and wherein said linker Z is —X-A-(B)ₚ-(CH₂)_{q}—Y, wherein X is O; A is R^{b}—C₁₋₇alkylene, wherein R^{b} is an optionally substituted aryl or an optionally substituted heteroaryl, and wherein r is 0 to 6, preferably r is 1, 2 or 3, and further preferably r is 1; B is NHC(O) or S; p is 1; q is 1 to 6, preferably q is 1, 2, 3 or 4, and further preferably q is 1, 2 or 3; Y is SH, N₃ or NH₂.

In a further preferred embodiment, said compound is a compound of formula (II), and wherein said linker Z is —X-A-(B)ₚ-(CH₂)_{q}—Y, wherein X is O; A is R^{b}—C₁₋₇alkylene, wherein R^{b} is an optionally substituted aryl or an optionally substituted heteroaryl, and wherein r is 0 to 6, preferably r is 1, 2 or 3, and further preferably r is 1; B is NHC(O) or S; p is 0 or 1; q is 0 to 6, preferably q is 0 to 4, and further preferably q is 0, 2 or 3; Y is SH, N₃ or NH₂. Further preferably, said compound is compound of formula 26*, 37*, 49*, 58*. Further preferably, said compound is compound of formula 26*, 37*, 56*, 58*. Again further preferably, said compound is a compound of formula 26* or a compound of formula 37*.

In a further preferred embodiment, said compound is a compound of formula (II), and wherein said linker Z is —X-A-(B)ₚ-(CH₂)_{q}—Y, wherein X is 0; A is R^{b}—C₁₋₇alkylene, wherein R^{b} is an optionally substituted aryl or an optionally substituted heteroaryl, and wherein r is 0 to 6, preferably r is 1, 2 or 3, and further preferably r is 1; B is NHC(O) or S; p is 1; q is 1 to 6, preferably q is 1, 2, 3 or 4, and further preferably q is 1, 2 or 3; Y is SH, N₃ or NH₂. Further preferably, said compound is compound of formula 26*, 37*, 49*, 58*. Further preferably, said compound is compound of formula 26*, 37*, 56*, 58*. Again further preferably, said compound is a compound of formula 26* or a compound of formula 37*.

In a further preferred embodiment, said compound is a compound of formula (III), and wherein said linker Z is —X-A-(B)ₚ-(CH₂)_{q}—Y, wherein X is O; A is C₁₋₇alkylene, C₁₋₄alkylene-(OCH₂CH₂)ᵣOC₁₋₄alkylene or R^{b}—C₁₋₇alkylene, wherein R^{b} is an optionally substituted aryl or an optionally substituted heteroaryl, and wherein r is 0 to 6, preferably r is 1, 2 or 3, and further preferably r is 1; B is NHC(O) or S; p is 0 or 1; q is 0 to 6, preferably q is 0 to 4, and further preferably q is 0, 2 or 3; Y is SH, N₃ or NH₂. Further preferably, said compound is compound of formula 8*, 31*, 34*, 37*, 45*, 47*, 50*-56*, 58*. Further preferably, said compound is compound of formula 8*, 31*, 34*, 45*, 47*, 49*-55*. Again further preferably, said compound is a compound of formula 8*, a compound of formula 31* or a compound of formula 34*.

In a further preferred embodiment, said compound is a compound of formula (III), and wherein said linker Z is —X-A-(B)ₚ-(CH₂)_{q}—Y, wherein X is O; A is C₁₋₇alkylene, C₁₋₄alkylene-(OCH₂CH₂)ᵣOC₁₋₄alkylene or R^{b}—C₁₋₇alkylene, wherein R^{b} is an optionally substituted aryl or an optionally substituted heteroaryl, and wherein r is 0 to 6, preferably r is 1, 2 or 3, and further preferably r is 1; B is NHC(O) or S; p is 1; q is 1 to 6, preferably q is 1, 2, 3 or 4, and further preferably q is 1, 2 or 3; Y is SH, N₃ or NH₂. Further preferably, said compound is compound of formula 8*, 31*, 34*, 37*, 45*, 47*, 50*-56*, 58*. Further preferably, said compound is compound of formula 8*, 31*, 34*, 45*, 47*, 49*-55*. Again further preferably, said compound is a compound of formula 8*, a compound of formula 31*or a compound of formula 34*.

In a further preferred embodiment, said compound is a compound of formula (III), and wherein said linker Z is —X-A-(B)ₚ-(CH₂)_{q}—Y, wherein X is O; A is R^{b}—C₁₋₇alkylene, wherein R^{b} is an optionally substituted aryl or an optionally substituted heteroaryl, and wherein r is 0 to 6, preferably r is 1, 2 or 3, and further preferably r is 1; B is NHC(O) or S; p is 0 or 1; q is 0 to 6, preferably q is 0 to 4, and further preferably q is 0, 2 or 3; Y is SH, N₃ or NH₂. Further preferably, said compound is compound of formula 8*, 31*, 34*, 37*, 45*, 47*, 50*-56*, 58*. Further preferably, said compound is compound of formula 8*, 31*, 34*, 45*, 47*, 49*-55*. Again further preferably, said compound is a compound of formula 8*, a compound of formula 31*or a compound of formula 34*.

In a further preferred embodiment, said compound is a compound of formula (III), and wherein said linker Z is —X-A-(B)ₚ-(CH₂)_{q}—Y, wherein X is O; A is R^{b}—C₁₋₇alkylene, wherein R^{b} is an optionally substituted aryl or an optionally substituted heteroaryl, and wherein r is 0 to 6, preferably r is 1, 2 or 3, and further preferably r is 1; B is NHC(O) or S; p is 1; q is 1 to 6, preferably q is 1, 2, 3 or 4, and further preferably q is 1, 2 or 3; Y is SH, N₃ or NH₂. Further preferably, said compound is compound of formula 8*, 31*, 34*, 37*, 45*, 50*-56*, 58*. Further preferably, said compound is compound of formula 8*, 31*, 34*, 45*, 47*, 49*-55*. Again further preferably, said compound is a compound of formula 8*, a compound of formula 31*or a compound of formula 34*.

In a further preferred embodiment, said compound is a compound of formula (IV), and wherein said linker Z is —X-A-(B)ₚ-(CH₂)_{q}—Y, wherein X is O; A is C₁₋₇alkylene, C₁₋₄alkylene-(OCH₂CH₂)ᵣOC₁₋₄alkylene or R^{b}—C₁₋₇alkylene, wherein R^{b} is an optionally substituted aryl or an optionally substituted heteroaryl, and wherein r is 0 to 6, preferably r is 1, 2 or 3, and further preferably r is 1; B is NHC(O) or S; p is 0 or 1; q is 0 to 6, preferably q is 0 to 4, and further preferably q is 0, 2 or 3; Y is SH, N₃ or NH₂. Further preferably, said compound is compound of formula 48*.

In a further preferred embodiment, said compound is a compound of formula (IV), and wherein said linker Z is —X-A-(B)ₚ-(CH₂)_{q}—Y, wherein X is O; A is C₁₋₇alkylene, C₁₋₄alkylene (OCH₂CH₂)ᵣOC₁₋₄alkylene or R^{b}—C₁₋₇alkylene, wherein R^{b} is an optionally substituted aryl or an optionally substituted heteroaryl, and wherein r is 0 to 6, preferably r is 1, 2 or 3, and further preferably r is 1; B is NHC(O) or S; p is 1; q is 1 to 6, preferably q is 1, 2, 3 or 4, and further preferably q is 1, 2 or 3; Y is SH, N₃ or NH₂. Further preferably, said compound is compound of formula 48*.

In a further preferred embodiment, said compound is a compound of formula (IV), and wherein said linker Z is —X-A-(B)ₚ-(CH₂)_{q}—Y, wherein X is O; A is R^{b}—C₁₋₇alkylene, wherein R^{b} is an optionally substituted aryl or an optionally substituted heteroaryl, and wherein r is 0 to 6, preferably r is 1, 2 or 3, and further preferably r is 1; B is NHC(O) or S; p is 0 or 1; q is 0 to 6, preferably q is 0 to 4, and further preferably q is 0, 2 or 3; Y is SH, N₃ or NH₂. Further preferably, said compound is compound of formula 48*.

In a further preferred embodiment, said compound is a compound of formula (IV), and wherein said linker Z is —X-A-(B)ₚ-(CH₂)_{q}—Y, wherein X is O; A is R^{b}—C₁₋₇alkylene, wherein R^{b} is an optionally substituted aryl or an optionally substituted heteroaryl, and wherein r is 0 to 6, preferably r is 1, 2 or 3, and further preferably r is 1; B is NHC(O) or S; p is 1; q is 1 to 6, preferably q is 1, 2, 3 or 4, and further preferably q is 1, 2 or 3; Y is SH, N₃ or NH₂. Further preferably, said compound is compound of formula 48*.

In a further preferred embodiment, said linker Z is of a formula selected from any one of the formula:

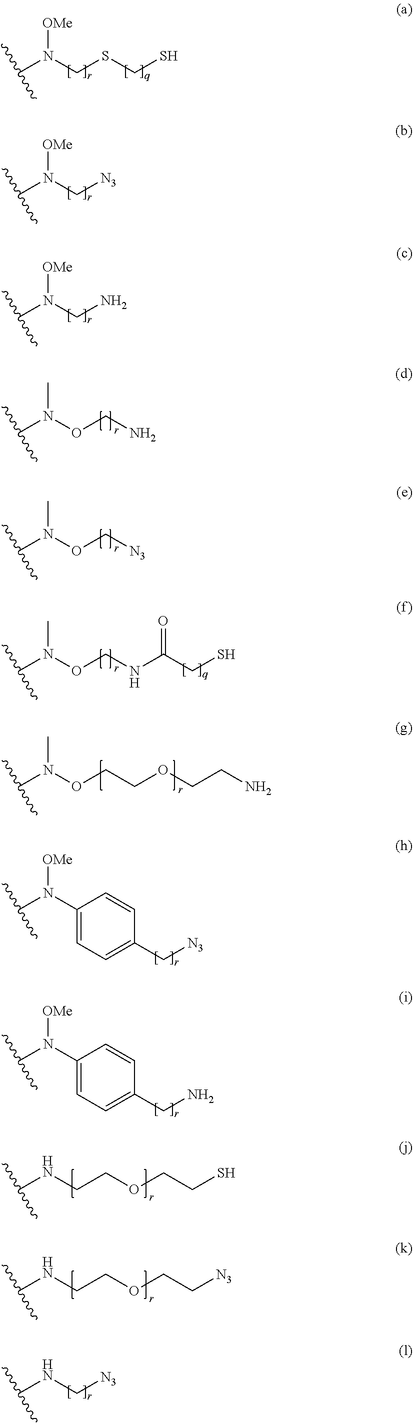

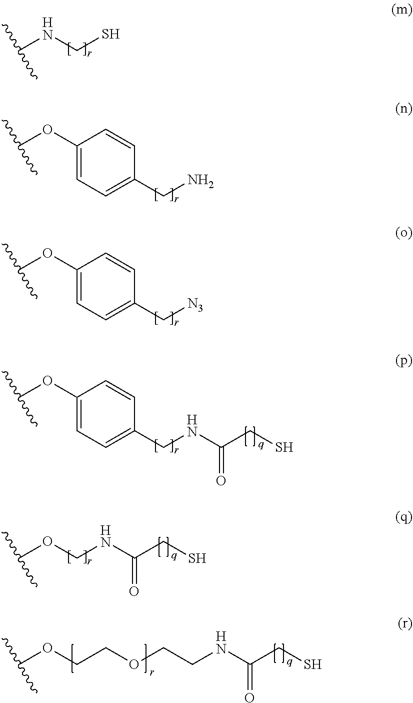

wherein r is 0 to 6, preferably 1 to 3, in particular 2 or 3, and q is 1 to 6, preferably 1 to 4, in particular 2 or 3.

In a further very preferred embodiment, said linker Z is formula (a)

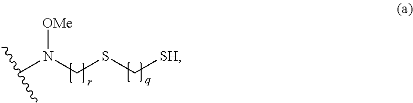

wherein r is 1 to 6, preferably 1 to 3, in particular 3, and q is 1 to 6, preferably 1, 2 and 3, in particular 2 or 3.

In a further preferred embodiment, said linker Z is formula (b)

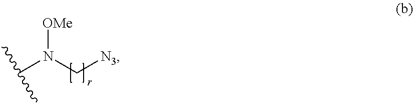

wherein r is 1 to 6, preferably 1 to 3, in particular 3.

In a further preferred embodiment, said linker Z is formula (c)

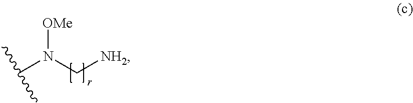

wherein r is 1 to 6, preferably 1 to 3, in particular 3.

In a further preferred embodiment, said linker Z is formula (d)

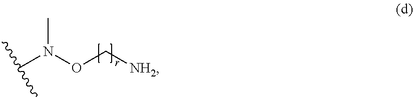

wherein r is 1 to 6, preferably 1 to 3, in particular 2.

In a further preferred embodiment, said linker Z is formula

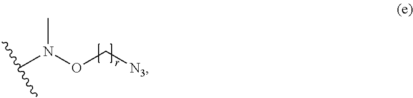

wherein r is 1 to 6, preferably 1 to 3, in particular 2.

In a further preferred embodiment, said linker Z is formula (f)

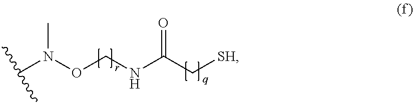

wherein r is 1 to 6, preferably 1 to 3, in particular 2, and q is 1 to 6, preferably 1, 2, 3 and 4, in particular 3.

In a further preferred embodiment, said linker Z is formula (g)

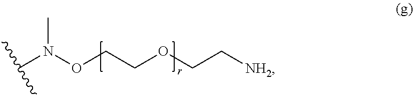

wherein r is 0 to 6, preferably 1 to 3, in particular 1.

In a further preferred embodiment, said linker Z is formula (h)

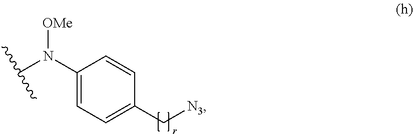

wherein r is 0 to 6, preferably 1 to 3, in particular 2.

In a further preferred embodiment, said linker Z is formula (i)

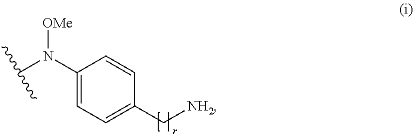

wherein r is 0 to 6, preferably 1 to 3, in particular 2.

In a further preferred embodiment, said linker Z is formula (j)

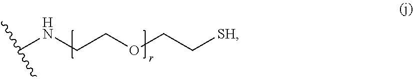

wherein r is 0 to 6, preferably 1 to 3, in particular 1.

In a further nreferreri emhrviiment. said linker Z is formula (k)

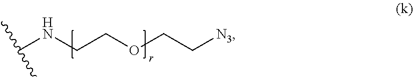

wherein r is 0 to 6, preferably 1 to 3, in particular 1.

In a further preferred embodiment, said linker Z is formula (l)

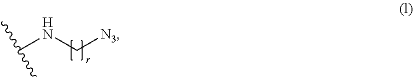

wherein r is 1 to 6, preferably 2 to 4, in particular 3.

In a further preferred embodiment, said linker Z is formula (m)

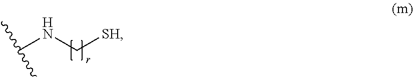

wherein r is 1 to 6, preferably 2 to 4, in particular 3.

In a further preferred embodiment, said linker Z is formula (n)

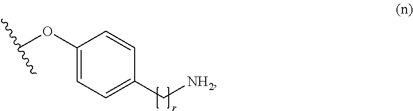

wherein r is 0 to 6, preferably 1 to 3, in particular 2.

In a further preferred embodiment, said linker Z is formula (o)

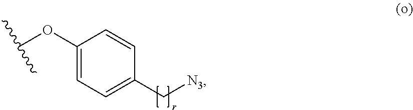

wherein r is 0 to 6, preferably 1 to 3, in particular 2.

In a further very preferred embodiment, said linker Z is formula (p)

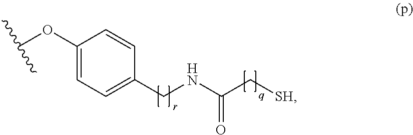

wherein r is 0 to 6, preferably 1 to 3, in particular 2, and q is 1 to 6, preferably 1, 2, 3 and 4, in particular 3.

In a further very preferred embodiment, said linker Z is formula (q)

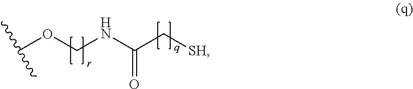

wherein r is 1 to 6, preferably 2 to 4, in particular 3, and q is 1 to 6, preferably 1, 2, 3 and 4, in particular 3.

In a preferred embodiment, said linker Z is of a formula selected from any one of the formula (a), (d), (l), (m), (n), (o), (p) or (q),

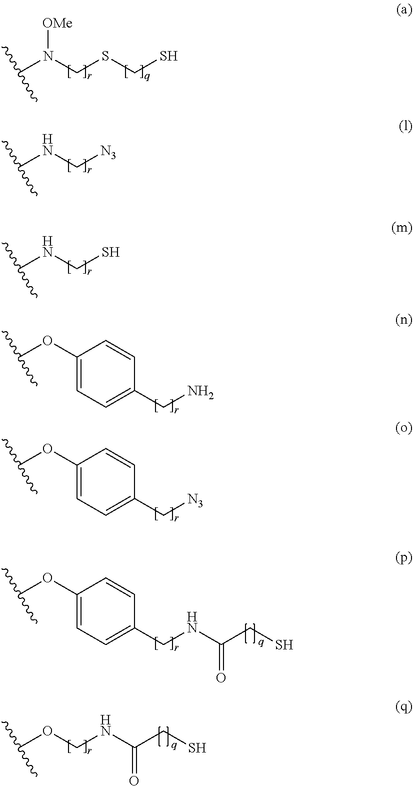

wherein r is 0 to 6, preferably 0 to 3, in particular 2 or 3, and q is 1 to 6, preferably 1, 2, 3 and 4, in particular 2 or 3.

In a further preferred embodiment, said linker Z is of a formula selected from any one of the formula (a), (p) or (q),

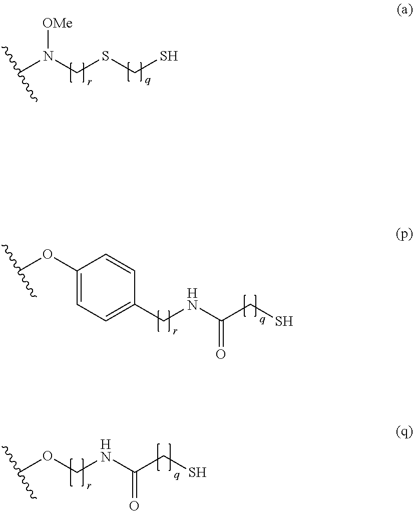

wherein r is 1 to 6, preferably 2 to 4, in particular 2 or 3, and q is 1 to 6, preferably 1, 2, 3 and 4, in particular 2 or 3.

In another preferred embodiment, said glycoepitope recognized by a CBP is selected from glycolipids such as the globo- and ganglio-types; red blood cell glycoantigens; the Tn and sialyl-Tn glycoantigens. In a further preferred embodiment, said glycoepitopes recognized by CBPs is a globoside, wherein preferably said globoside is selected from Gb3, Gb4. In a further preferred embodiment, said glycoepitopes recognized by CBPs is a red blood cell glycoantigen, wherein preferably said antigen is selected from the A antigen, B antigen, I antigen, P antigen, Lewis^{a} antigen, Lewis^{b} antigen, Lewis^{x} antigen, Lewis^{y} antigen. In a further preferred embodiment, said glycoepitopes recognized by CBPs is the Tn antigen and the sialyl-Tn antigen. In a further preferred embodiment, said glycoepitopes recognized by CBPs is a ganglioside, wherein preferably said ganglioside is selected from GM1a, GM1b, asialo GM1, GD1a, GD1b, GT1a, GT1b, GQ1b, asialo GM2, GM2, GD2, GM3, GD3.

In a further preferred embodiment, said carbohydrate moiety mimicking, or alternatively and preferably being, a glycoepitope recognized by a CBP is a carbohydrate moiety comprised by a compound of formula (I), and said glycoepitope is a glycoepitope of the globo-type.

In a further preferred embodiment, said carbohydrate moiety mimicking, or alternatively and preferably being, a glycoepitope recognized by a CBP is a carbohydrate moiety comprised by a compound of formula (II), and said glycoepitope is a glycoepitope of the Tn antigen or sialyl-Tn antigen.

In a further preferred embodiment, said carbohydrate moiety mimicking, or alternatively and preferably being, a glycoepitope recognized by a CBP is a carbohydrate moiety comprised by a compound of formula (III) and said glycoepitope is a glycoepitope of the A antigen, B antigen, I antigen, i antigen, P antigen and Lewis antigen system.

In a further preferred embodiment, said carbohydrate moiety mimicking, or alternatively and preferably being, a glycoepitope recognized by a CBP is a carbohydrate moiety comprised by a compound of formula (IV), and said glycoepitope is a glycoepitope of the ganglio-type.

In a further very preferred embodiment, said compound is a compound of formula 3, 8, 22 26, 31, 34, 37, 45 or 48:

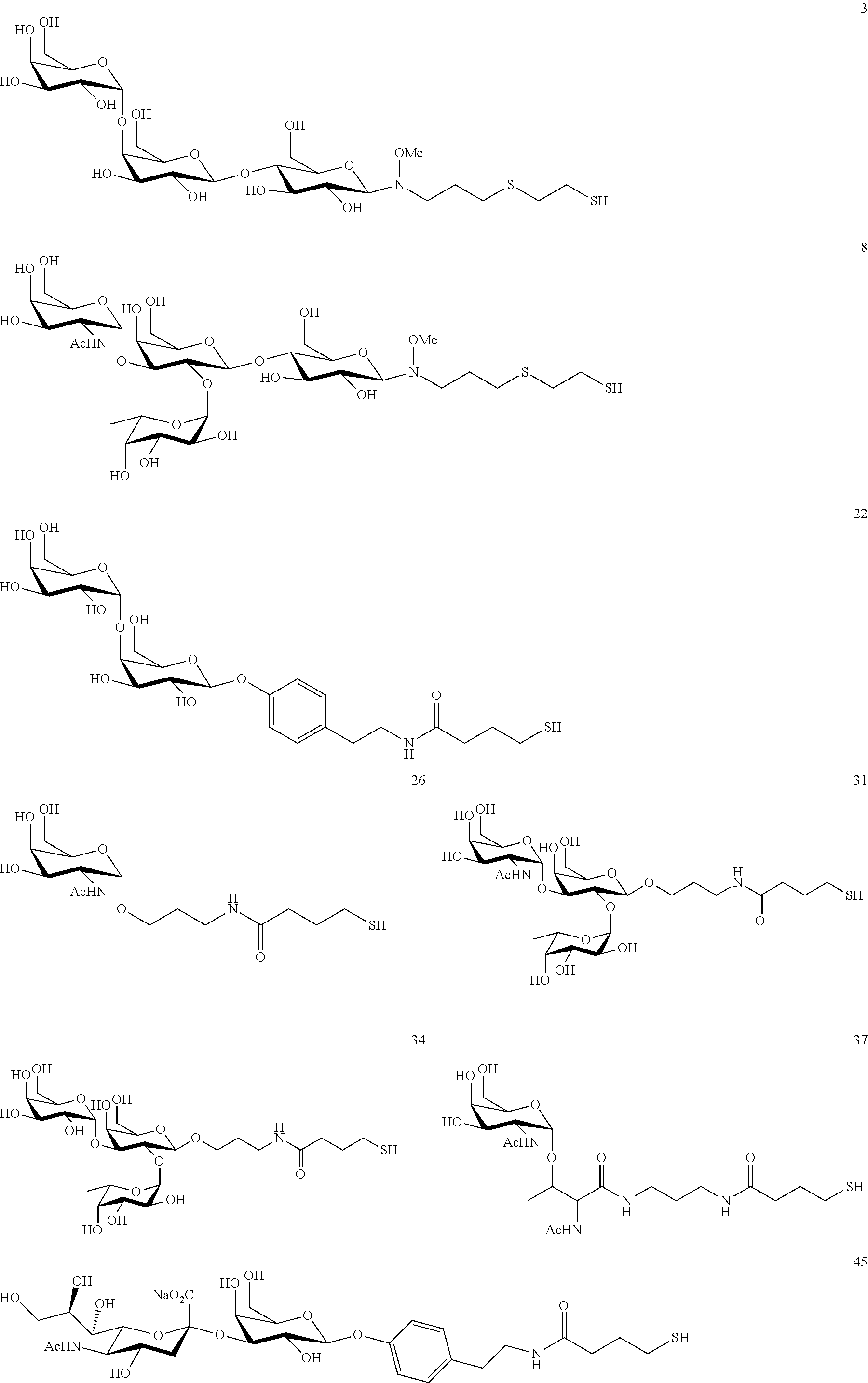

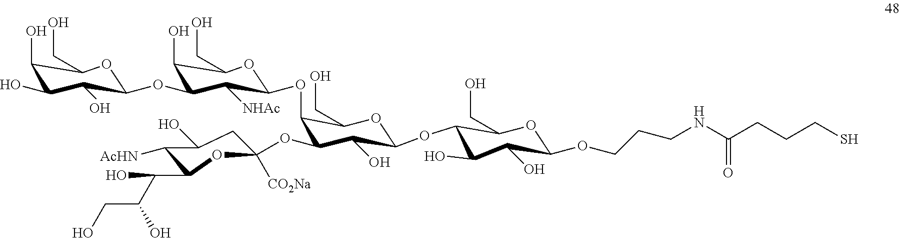

In a further very preferred embodiment, said compound is a compound of formula 3. In a further very preferred embodiment, said compound is a compound of formula 8. In a further very preferred embodiment, said compound is a compound of formula 22. In a further very preferred embodiment, said compound is a compound of formula 26. In a further very preferred embodiment, said compound is a compound of formula 31. In a further very preferred embodiment, said compound is a compound of formula 34. In a further very preferred embodiment, said compound is a compound of formula 37. In a further very preferred embodiment, said compound is a compound of formula 45. In a further very preferred embodiment, said compound is a compound of formula 48.

In a further preferred embodiment, said compound is a compound of formula (I), and wherein said linker Z is —X-A-(B)ₚ-(CH₂)_{q}—Y, wherein X is N(R^{a}); R^{a} is H, C₁₋₄-alkyl, C₁₄-alkoxy, CH₂C₆H₅, CH₂CH₂C₆H₅, OCH₂C₆H₅, or OCH₂CH₂C₆H₅; A is C₁₋₇alkylene, OC₁₋₇alkylene C₁₋₄alkylene-(OCH₂CH₂)ᵣOC₁₋₄alkylene, OC₁₋₇alkylene-R^{b}, or R^{b}—C₁₋₇alkylene wherein R^{b} is an optionally substituted aryl or an optionally substituted heteroaryl, and wherein r is 0 to 6, preferably r is 1, 2 or 3, and further preferably r is 1; B is NHC(O) or S; p is 0 or 1; q is 0 to 6, preferably q is 0 to 4, and further preferably q is 0, 2 or 3; Y is SH, N₃ or NH₂. Further preferably, said compound is compound of formula 3*, 22*, 57*. Again further preferably, said compound is a compound of formula 3* and 57*. And again further preferably, said compound is a compound of formula 3.

In a further preferred embodiment, said compound is a compound of formula (III), and wherein said linker Z is —X-A-(B)ₚ-(CH₂)_{q}—Y, wherein X is N(R^{a}); R^{a} is H, C₁₋₄-alkyl, C₁₋₄-alkoxy, CH₂C₆H₅, CH₂CH₂C₆H₅, OCH₂C₆H₅, or OCH₂CH₂C₆H₅; A is C₁₋₇alkylene, OC₁₋₇alkylene C₁₋₄alkylene-(OCH₂CH₂)ᵣOC₁₋₄alkylene, OC₁₋₇alkylene-R^{b}, or R^{b}—C₁₋₇alkylene wherein R^{b} is an optionally substituted aryl or an optionally substituted heteroaryl, and wherein r is 0 to 6, preferably r is 1, 2 or 3, and further preferably r is 1; B is NHC(O) or S; p is 0 or 1; q is 0 to 6, preferably q is 0 to 4, and further preferably q is 0, 2 or 3; Y is SH, N₃ or NH₂. Further preferably, said compound is compound of formula 8*, 31*, 34*, 37*, 45*, 47*, 50*-56*, 58*. Further preferably, said compound is compound of formula 8*, 31*, 34*, 45*, 47*, 49*-55*. Again further preferably, said compound is compound of formula 8*, 31*, 34*, 50*, 51*, 54*. Again further preferably, said compound is compound of formula 8*, 31* or 34*. Again further preferably, said compound is a compound of formula 8*. Alternatively further preferably, said compound is a compound of formula 31*. Alternatively further preferably, said compound is a compound of formula 34*. And again further preferably, said compound is a compound of formula 8, 31 or 34. Very preferably, said compound is a compound of formula 8. Alternatively very preferably, said compound is a compound of formula 31. Alternatively very preferably, said compound is a compound of formula 34.

In a further preferred embodiment, said compound is a compound of formula (I), and wherein said linker Z is —X-A-(B)ₚ-(CH₂)_{q}—Y, wherein X is 0; A is C₁₋₇alkylene, C₁₋₄alkylene-(OCH₂CH₂)ᵣOC₁₋₄alkylene or R^{b}—C₁₋₇alkylene, wherein R^{b} is an optionally substituted aryl or an optionally substituted heteroaryl, and wherein r is 0 to 6, preferably r is 1, 2 or 3, and further preferably r is 1; B is NHC(O) or S; p is 0 or 1; q is 0 to 6, preferably q is 0 to 4, and further preferably q is 0, 2 or 3; Y is SH, N₃ or NH₂. Further preferably, said compound is compound of formula 3*, 22*, 57*. Again further preferably, said compound is a compound of formula 22*. And again further preferably, said compound is a compound of formula 22.

In a further preferred embodiment, said compound is a compound of formula (I), and wherein said linker Z is —X-A-(B)ₚ-(CH₂)_{q}—Y, wherein X is O; A is C₁₋₇alkylene, C₁₋₄alkylene-(OCH₂CH₂)ᵣOC₁₋₄alkylene or R^{b}—C₁₋₇alkylene, wherein R^{b} is an optionally substituted aryl or an optionally substituted heteroaryl, and wherein r is 0 to 6, preferably r is 1, 2 or 3, and further preferably r is 1; B is NHC(O) or S; p is 1; q is 1 to 6, preferably q is 1, 2, 3 or 4, and further preferably q is 1, 2 or 3; Y is SH, N₃ or NH₂. Further preferably, said compound is compound of formula 3*, 22*, 57*. Again further preferably, said compound is a compound of formula 22*. And again further preferably, said compound is a compound of formula 22.

In a further preferred embodiment, said compound is a compound of formula (I), and wherein said linker Z is —X-A-(B)ₚ-(CH₂)_{q}—Y, wherein X is O; A is R^{b}—C₁₋₇alkylene, wherein R^{b} is an optionally substituted aryl or an optionally substituted heteroaryl, and wherein r is 0 to 6, preferably r is 1, 2 or 3, and further preferably r is 1; B is NHC(O) or S; p is 0 or 1; q is 0 to 6, preferably q is 0 to 4, and further preferably q is 0, 2 or 3; Y is SH, N₃ or NH₂. Further preferably, said compound is compound of formula 3*, 22*, 57*. Again further preferably, said compound is a compound of formula 22*. And again further preferably, said compound is a compound of formula 22.

In a further preferred embodiment, said compound is a compound of formula (I), and wherein said linker Z is —X-A-(B)ₚ-(CH₂)_{q}—Y, wherein X is O; A is R^{b}—C₁₋₇alkylene, wherein R^{b} is an optionally substituted aryl or an optionally substituted heteroaryl, and wherein r is 0 to 6, preferably r is 1, 2 or 3, and further preferably r is 1; B is NHC(O) or S; p is 1; q is 1 to 6, preferably q is 1, 2, 3 or 4, and further preferably q is 1, 2 or 3; Y is SH, N₃ or NH₂. Further preferably, said compound is compound of formula 3*, 22*, 57*. Again further preferably, said compound is a compound of formula 22*. And again further preferably, said compound is a compound of formula 22.

In a further preferred embodiment, said compound is a compound of formula (II), and wherein said linker Z is —X-A-(B)ₚ-(CH₂)_{q}—Y, wherein X is O; A is C₁₋₇alkylene, C₁₋₄alkylene-(OCH₂CH₂)ᵣOC₁₋₄alkylene or R^{b}—C₁₋₇alkylene, wherein R^{b} is an optionally substituted aryl or an optionally substituted heteroaryl, and wherein r is 0 to 6, preferably r is 1, 2 or 3, and further preferably r is 1; B is NHC(O) or S; p is 0 or 1; q is 0 to 6, preferably q is 0 to 4, and further preferably q is 0, 2 or 3; Y is SH, N₃ or NH₂. Further preferably, said compound is compound of formula 26*, 37*, 49*, 58*. Further preferably, said compound is compound of formula 26*, 37*, 56*, 58*. Again further preferably, said compound is a compound of formula 26* or a compound of formula 37*. Again further preferably, said compound is a compound of formula 26*. Alternatively further preferably said compound is a compound of formula 37*. And again further preferably, said compound is a compound of formula 26 or a compound of formula 37. Very preferably said compound is a compound of formula 26. Alternatively very preferably said compound is a compound of formula 37.

In a further preferred embodiment, said compound is a compound of formula (II), and wherein said linker Z is —X-A-(B)ₚ-(CH₂)_{q}—Y, wherein X is O; A is C₁₋₇alkylene, C₁₋₄alkylene (OCH₂CH₂)ᵣOC₁₋₄alkylene or R^{b}—C₁₋₇alkylene, wherein R^{b} is an optionally substituted aryl or an optionally substituted heteroaryl, and wherein r is 0 to 6, preferably r is 1, 2 or 3, and further preferably r is 1; B is NHC(O) or S; p is 1; q is 1 to 6, preferably q is 1, 2, 3 or 4, and further preferably q is 1, 2 or 3; Y is SH, N₃ or NH₂. Further preferably, said compound is compound of formula 26*, 37*, 49*, 58*. Further preferably, said compound is compound of formula 26*, 37*, 56*, 58*. Again further preferably, said compound is a compound of formula 26*. Alternatively further preferably said compound is a compound of formula 37*. And again further preferably, said compound is a compound of formula 26 or a compound of formula 37. Very preferably said compound is a compound of formula 26. Alternatively very preferably said compound is a compound of formula 37.

Furthermore, the invention relates to therapeutically acceptable, typically and preferably biodegradable, polymers comprising a multitude of substituents, wherein said compounds are connected to the polymer backbone by way of the linker Z, and wherein the connection is effected via the Y-moiety of linker Z. Typically and preferably, said inventive polymer further comprises spacer moieties for coupling of said Y-moieties of the linker Z to reactive moieties on the polymer backbone. Such spacer moieties are known to the skilled person in the art and preferred examples are described herein. In some embodiments, said Ymoieties of the linker Z are directly linked to reactive moieties on the polymer backbone without a further spacer, however.

Thus, in another aspect, the present invention provides for a polymer comprising a multitude of the inventive compounds, wherein said compounds are connected to the polymer backbone by way of said linker Z, and wherein said connection is effected via the Y-group of said linker Z, and said inventive polymer further optionally comprises a spacer moiety for coupling of said Y-moiety of the linker Z to a reactive moiety on the polymer backbone. Preferred examples are described herein.

In a further aspect, the present invention provides for a polymer comprising (i) a multitude of compounds of formula (I), (ii) a multitude of compounds of formula (II), (iii) a multitude of compounds of formula (III), (iv) a multitude of compounds of formula (IV) or (v) a multitude of compounds of formula (I), formula (II), formula (III) and formula (IV), wherein said compounds are connected to the polymer backbone by way of said linker Z, and wherein said connection is effected via the Y-group of said linker Z. Preferably said multitude of compounds of any one of formula (I) , formula (II), formula (III), formula (IV) are either identical compounds of formula (I),formula (II), formula (III), formula (IV) or different compounds selected independently from of formula (I), formula (II), formula (III), formula (IV). Typically, said inventive polymer further optionally comprises spacer moieties for coupling of said Ymoieties of the linker Z to reactive moieties on the polymer backbone. Preferred examples are described herein.

The invention further particularly relates to therapeutically acceptable polymers comprising independently of a multitude of any one of the compounds of formula (I), (II), (III) and (IV), including polymers with loading of a multitude of one identical compound of formula (I), (II), (III) or (IV) or a multitude being a combination of several different compounds of formula (I) (II), (III) or (IV). Preferred polymers in said context are polymers with loading of one or several of compounds of formula (I), (II), (III) or (IV), wherein said compounds of formula (I),

(II), (III) or (IV) are preferably selected from 3*, 8*, 22*, 26*, 31*, 34*, 37*, 45*, 47*-58*. In another preferred embodiment, the polymers of the invention are polymers with loading of one or several of identical compounds independently selected from any one of the formula (I), (II), (III) or (IV), wherein said compounds of formula (I), (II), (III) or (IV) are preferably selected from 3*, 8*, 22*, 26*, 31*, 34*, 37*, 45*, 47*-58*.

The inventive polymer comprising the multitude of identical or different, preferably of identical, compounds of formula (I) and/or (II) and/or (III) and/or (IV) wherein the Y-group of said linker Z connects said compounds to the polymer backbone, is preferably an α-amino acid polymer, and hereby typically and preferably a homomeric or heteromeric α-amino acid polymer, an acrylic acid or methacrylic acid polymer or copolymer, or a N-vinyl-2-pyrrolidone-vinylalcohol copolymer, a chitosan polymer, or a polyphosphazene polymer.

In a further preferred embodiment, said polymer backbone is an α-amino acid polymer, an acrylic acid or methacrylic acid polymer or copolymer, a N-vinyl-2-pyrrolidone-vinyl alcohol copolymer, a chitosan polymer, or a polyphosphazene polymer, wherein preferably said polymer backbone is an α-amino acid polymer, and wherein further preferably said α-amino acid of said α-amino acid polymer is lysine, ornithine, glutamine, asparagine, glutamic acid or aspartic acid.

In a further preferred embodiment, said multitude of a compound is a number of said compounds of 10 to 1000, preferably 20-700, further preferably, 50-300. In a further preferred embodiment, said multitude of a compound is a number of said compounds of 20-700. In a further preferred embodiment, said multitude of a compound is a number of said compounds of 50-300.

In a preferred embodiment, the polymer backbone is an α-amino acid polymer, an acrylic acid or methacrylic acid polymer or copolymer, a N-vinyl-2-pyrrolidone-vinyl alcohol copolymer, a chitosan polymer, or a polyphosphazene polymer.

In another preferred embodiment, the polymer backbone is an α-amino acid polymer.

In a further preferred embodiment, the polymer backbone is an α-amino acid polymer and said α-amino acid of said α-amino acid polymer is lysine, ornithine, glutamine, asparagine, glutamic acid, aspartic acid or serine.

In a further very preferred embodiment, said polymer backbone is an α-amino acid polymer, wherein said α-amino acid of said α-amino acid polymer is lysine, and wherein further preferably said poly-lysine is a biodegradable poly-lysine

In a very preferred embodiment, the polymer backbone is poly-lysine, preferably poly-L-lysine, and wherein preferably the molecular weight of said poly-lysine is 1,000 Da to 300,000 Da.

In a very preferred embodiment, the polymer backbone is poly-lysine, preferably poly-L-lysine, and wherein preferably the molecular weight of said poly-lysine is 30,000 Da to 150,000 Da.

In a further very preferred embodiment, the polymer backbone is a poly-L-lysine.

In a further very preferred embodiment, the polymer backbone is a biodegradable polymer backbone. In again a further very preferred embodiment, the polymer backbone is biodegradable polylysine, preferably a biodegradable poly-L-lysine.

In a further preferred embodiment, the percentage of loading of the carbohydrate moiety of said compound onto the polymer backbone is between 10 and 90%, preferably between 20 and 70%, and in particular between 30 and 60%. The latter means that 30 to 60% of the reactive polymer side chains and, if applicable the spacer moiety, are reacted with the —Y group of said linker Z. The percentage of loading of the carbohydrate moiety of said compound onto the polymer backbone is typically and preferably determined by NMR spectroscopy and refers to % mole/mole.

Further particular and preferred examples of polymers of the invention are depicted schematically in Table 1 and are described thereafter.

The introduction of the spacer, if present within the polymers of the present invention is typically and preferably effected by a first reaction of the polymer backbone with said spacer, which then is reacted with the Y moiety of the linker Z of the compound. In some embodiments, the introduction of the spacer, if present within the polymers of the present invention is effected by first a reaction of said spacer with the Y moiety of the linker Z of the compound, which then is reacted with the polymer backbone.

TABLE 1
Preferred examples of inventive polymers

| Example | Polymer backbone | Compound | Polymer |
|---|---|---|---|
| (A) | 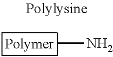 | 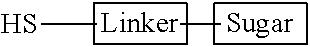 | 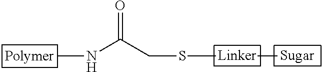 |
| | | | 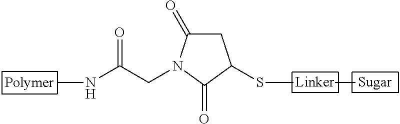 |
| | | 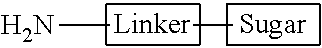 | 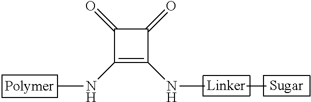 |
| | | 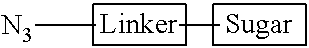 | 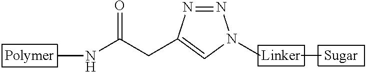 |
| (B) | 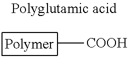 | 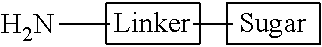 | 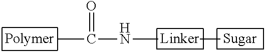 |
| (C) | 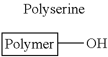 | 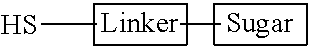 | 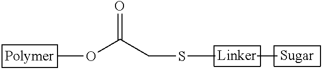 |
| (D) | 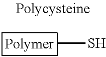 | 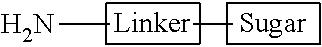 | 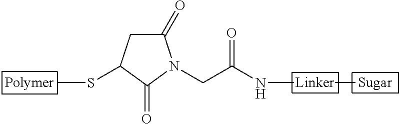 |
| (F) | 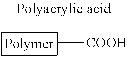 | 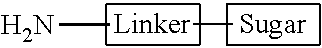 | 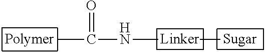 |
| (G) | 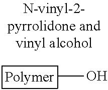 | 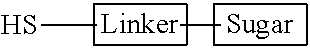 | 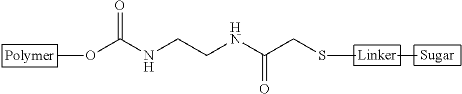 |
| (H) | 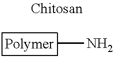 | 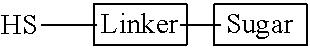 | 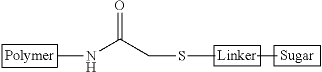 |
| (I) | 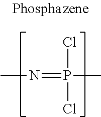 | 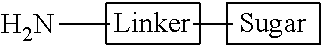 | 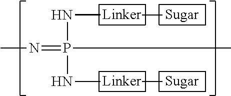 |

(A) a poly-α-amino acid, wherein the amino acid carries a side chain aminoalkyl function, such as in typically and preferably poly-lysine, in particular poly-L-lysine or poly-D-lysine, and the amino group is connected via a spacer moiety to the Y-group of said linker Z. In the case wherein Y is SH, a typical and preferred spacer moiety comprises a CH₂-group, typically and preferably a terminal CH₂-group, wherein said CH₂-group of said spacer moiety is connected to the S-group of said linker Z. A preferred spacer moiety is an acetyl group. Another preferred spacer moiety comprises a succinimide-group, typically and preferably a terminal succinimide-group, wherein said succinimide-group of said spacer moiety is connected to the S-group of said linker Z. In the case wherein Y is NH₂, a typical and preferred spacer moiety comprises a squaric acid diamide-group, typically and preferably a terminal squaric acid diamide-group, wherein said electrophilic carbon of said spacer moiety is connected to the NH-group of said linker Z. In the case wherein Y is N₃, a typical and preferred spacer moiety comprises an alkyne-group, typically and preferably a terminal alkyne-group, wherein said akyne-group of said spacer moiety is connected to the N₃-group of said linker Z via azide-alkyne cycloaddition.

(B) a poly-α-amino acid (D- and L-form), wherein the amino acid carries a side chain carbonylalkyl function, such as in typically and preferably poly-aspartic acid, poly-glutamic acid, poly-asparagine or poly-glutamine, and the carbonyl group (which corresponds to the original carboxy group in aspartic acid and glutamic acid, respectively) is connected to the Y-group of said linker Z. In the case wherein Y is NH₂, the carbonyl group is connected to the amine-group of said linker Z typically and preferably as an amide. In the case wherein Y is N₃, a typical and preferred spacer moiety comprises an alkyne group, typically and preferably a terminal alkyne-group, wherein said alkyne-group of said spacer moiety is connected to the N₃-group of said linker Z via azide-alkyne cycloaddition.

(C) a poly-α-amino acid (D- and L-form), wherein the amino acid carries a side chain hydroxyalkyl or hydroxyaryl function, such as in typically and preferably poly-serine, poly-threonine, poly-tyrosine, or poly-hydroxyproline, and the hydroxy group is connected via a spacer moiety to the Y-group of said linker Z. Exemplary spacer moieties include but are not limited to moieties comprising a terminal CH₂-group, wherein said terminal CH₂-group of said spacer moiety is connected to the S-group of said linker Z.

(D) a poly-α-amino acid, wherein the amino acid carries a side chain thiolalkyl function, such as in typically and preferably poly-cysteine, wherein the thiol group is connected via a spacer moiety to the terminal Y-group of linker Z. In the case wherein Y is NH₂, a typical and preferred spacer moiety comprises a terminal carbonyl-group, wherein said terminal carbonyl-group of said spacer moiety is connected to the NH-group of said linker Z;

(E) Co-polymers of two or more different α-amino acids connected typically and preferably via a spacer moiety, to the Y-group of said linker Z, as described in (A)-(D);

(F) poly-acrylic acid, poly-methacrylic acid or a copolymer of acrylic and methacrylic acid, wherein the carboxy group is connected to the Y-group of said linker Z. In the case wherein Y is NH₂, the carbonyl group is connected to the amine-group of said linker Z typically and preferably as a amide;

(G) a copolymer of N-vinyl-2-pyrrolidone and vinyl alcohol, wherein the hydroxy group of the vinyl alcohol part of the copolymer is connected via a spacer moiety to the Y-group of said linker Z. In the case wherein Y is SH, a typical and preferred spacer moiety comprises a terminal CH₂-group, wherein said terminal CH₂-group of said spacer moiety is connected to the S— of said linker Z. Exemplary spacer moieties include but are not limited to moieties comprising a terminal CH₂-group, wherein said terminal CH₂-group of said spacer moiety is connected to the S-group of said linker Z.

(H) chitosan, wherein the amino group is connected via a spacer moiety to the Y-group of said linker Z. In the case wherein Y is SH, a typical and preferred spacer moiety comprises a terminal CH₂-group, wherein said terminal CH₂-group of said spacer moiety is connected to the S-group of said linker Z. A preferred spacer moiety is an acetyl group. Another preferred spacer moiety comprises a terminal succinimide-group, wherein said terminal succinimide-group of said spacer moiety is connected to the S-group of said linker Z. In the case wherein Y is NH₂, a typical and preferred spacer moiety comprises a terminal squaric acid amide ester-group, wherein said terminal ester-group of said spacer moiety is connected to the NH group of said linker Z. In the case wherein Y is N₃, a typical and preferred spacer moiety comprises a terminal alkyne-group, wherein said terminal akyne-group of said spacer moiety is connected to the N₃-group of said linker Z via azide-alkyne cycloaddition.

(I) a polyphosphazene polymer, wherein the phosphorus is connected to the Y-group of said linker Z. In the case wherein Y is NH₂, the phophorus is connected to the amine-group of said linker Z.

In a particular embodiment, a polymer (A) comprises the partial formula (V)

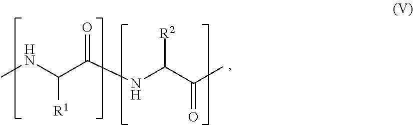

wherein

R¹ is an aminoalkyl substituent connected to said linker Z, wherein the Y-group of said linker

Z is connected to the terminal amino group of R¹ via a spacer moiety, wherein typically and preferably said spacer moiety is an acetyl group, a squaric acid group, succinimide group or alkyne, wherein preferably said spacer moiety is an acetyl group.

R² is 2,3-dihydroxypropyl substituent, which is a capped amino function having a solubilizing substituent,

and the relation between the two bracketed entities with R¹ and R², respectively, in the polymer indicates the relation of carbohydrate loading to capped amino function.

For example, R¹ is of formula (Va), (Vb), (Vc), (Vd)

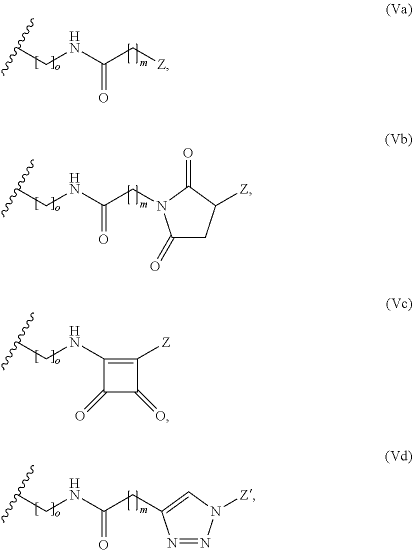

wherein Z′ is —X-A-(B)ₚ-(CH₂)_{q}—. as a consequence of the reaction of an alkyne group with said azide group (Y═N₃) of said linker Z.

and R² is of formula (Ve), (Vf), (Vg)

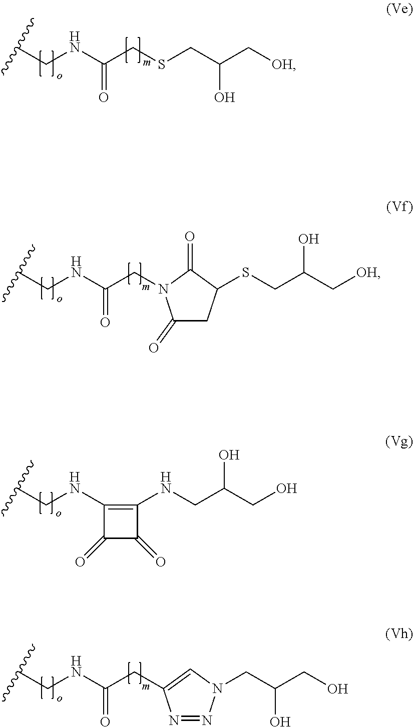

wherein o is between 1 and 6, preferably 3 or 4 and m is between 1 and 6, preferably between 1 and 2, in particular 1.

When o is 3, substituent R¹ represents a side chain of poly-ornithine, and when o is 4, substituent R¹ represents a side chain of poly-lysine, connected to said Y-group of said linker Z which linker Z is comprised by the inventive compounds, and preferably by the inventive compounds of formula (I), (II), (III) or (IV).

The poly-amino acid can be linear, hyperbranched or dendritic, as described by Z. Kadlecova et al., Biomacromolecules 2012, 13:3127-3137 and K. T. Al-Jamal et al., Journal of Drug Targeting 2006, 14:405-412, for poly-lysine as follows:

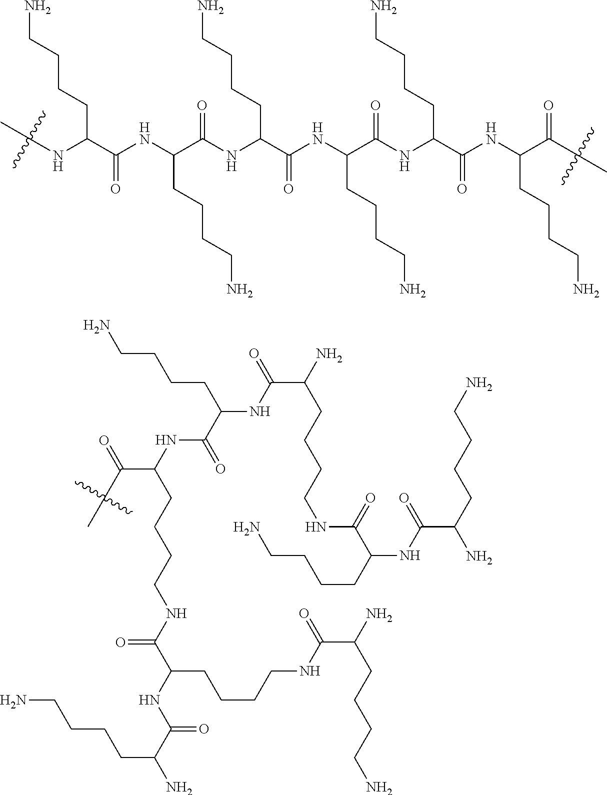 [SMILES: *NC(CCCCN)C(=O)NC(CCCCN)C(=O)NC(CCCCN)C(=O)NC(CCCCN)C(=O)NC(CCCCN)C(=O)NC(CCCCN)C(*)=O ; *C(=O)C(CCCCNC(=O)C(CCCCNC(=O)C(N)CCCCN)NC(=O)C(N)CCCCN)NC(=O)C(CCCCN)NC(=O)C(N)CCCCNC(=O)C(CCCCN)NC(=O)C(N)CCCCN]

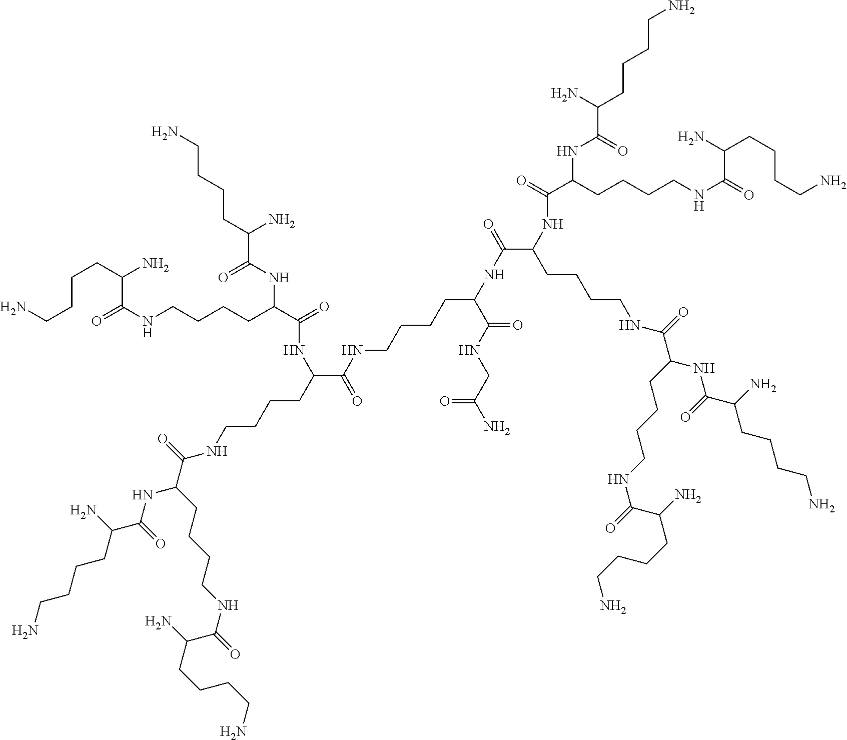 [SMILES: NCCCCC(N)C(=O)NCCCCC(NC(=O)C(N)CCCCN)C(=O)NCCCCC(NC(=O)C(CCCCNC(=O)C(N)CCCCN)NC(=O)C(N)CCCCN)C(=O)NCCCCC(NC(=O)C(CCCCNC(=O)C(CCCCNC(=O)C(N)CCCCN)NC(=O)C(N)CCCCN)NC(=O)C(CCCCNC(=O)C(N)CCCCN)NC(=O)C(N)CCCCN)C(=O)NCC(N)=O]

The poly-lysine used to prepare polymer (A) of formula (V) has preferably a molecular weight between 1,000 and 300,000 Da, in particular 30,000 to 150,000 Da, and such polymers further connected via the Y═SH group of the linker Z to compounds of formula (I) and/or (II) and/or (III) and/or (IV) and with a capping 2,3-dihydroxypropylthio-acetylylaminoalkyl residue are preferred. For example, the polylysine polymer is first functionalized by chloroacetylation. Reaction of the chloroacetylated polymer with said linker Z comprising the terminal thiol functionality by nucleophilic substitution gives access to the desired polymers.

In a particular embodiment, a polymer (B) comprises the partial formula (V)

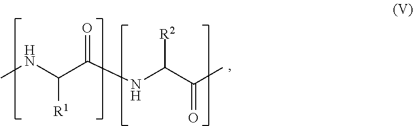

wherein

R¹ is a carbonylalkyl substituent connected to said linker Z, wherein the Y-group of said linker Z is connected to the carbonyl-group of R¹. Typically and preferably, said Y is NH₂, and the carbonyl group is directly connected to said amine group of said linker Z by forming an amide bond.

R² is 2,3-dihydroxypropylaminoacetyl-alkyl,

and the relation between the two bracketed entities with R¹ and R², respectively, in the polymer indicates the relation of carbohydrate loading to capped carbonyl or carboxy function.

For example, R¹ is of formula (Vi)

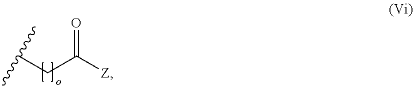

and R² is of formula (Vj)

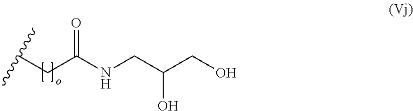

wherein o is between 1 and 6, preferably 1 or 2.

When o is 1 substituent R¹ represents a side chain of poly-asparagine, and when o is 2, substituent R¹ represents a side chain of poly-glutamine, connected to said Y-group preferably to said Y═NH₂ group, of said linker Z which linker Z is comprised by the inventive compounds, and preferably by the inventive compounds of formula (I), (II), (III) or (IV), and R² is 2,3-dihydroxy-carbonylalkyl, i.e. a capped amide function having a solubilizing substituent.

The poly-aspartic acid used to prepare polymer (B) of formula (V) has preferably a molecular weight between 1,000 and 300,000 Da, in particular 30,000 to 100,000 Da, and such polymers further connected via the Y-group of said linker Z to compounds of formula (I) and/or (II) and/or (III) and/or (IV) and with a capping 2,3-dihydroxypropylaminoacetyl-alkyl residue are preferred. For example, polyaspartic acid is directly coupled to said linker Z comprising the terminal amine functionality by amide formation gives access to the desired polyasparagine polymers.

In case of poly-aspartic acid or poly-glutamic acid the polymer can be linear, hyperbranched or dendritic.

In a particular embodiment, a polymer (C) comprises the partial formula (V)

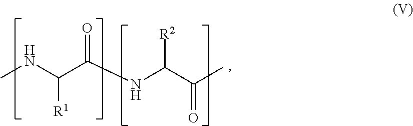

wherein

R¹ is a hydroxyalkyl or hydroxyaryl substituent connected to said linker Z, wherein the SH-group of said linker Z is connected to the —CH₂-group of R¹,

R² is 2,3-dihydroxypropylthioacetyl-hydroxyalkyl (or -hydroxyaryl),

and the relation between the two bracketed entities with R¹ and R², respectively, in the polymer indicates the relation of carbohydrate loading to capped hydroxy function.

For example, in the case of poly-serine and analogs, R¹ is of formula (Vk)

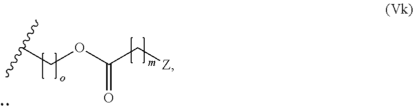

and R² is of formula (Vm)

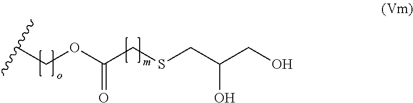

wherein o is between 1 and 6, preferably 1 or 2, in partcular 1, m is between 1 and 6, preferably between 1 and 2, in particular 1.

When o is 1, substituent R¹ represents a side chain of poly-serine, connected to said Y-group, preferably to said Y═SH group, of said linker Z, which linker Z is comprised by the inventive compounds, and preferably by the inventive compounds of formula (I) or (II) or (III) or (IV), and R² is 2,3-dihydroxypropylthio-hydroxyalkyl, i.e. a capped hydroxy function having a solubilizing substituent.

The poly-serine (and other hydroxy-functionalized α-amino acid side-chains) used to prepare polymer (C) of formula (V) has preferably a molecular weight between 1,000 and 300,000 Da, in particular 30,000 to 70,000 Da, and such polymers further connected via the Y-group of said linker Z to compounds of formula (I) and/or (II) and/or (III) and/or (IV) and with a capping 2,3-dihydroxypropylthio-hydroxyalkyl residue are preferred. For example, polyserine is first functionalized by chloroacetylation of the hydroxyl groups. Reaction of the chloroacetylated polymer with said linker Z comprising the terminal thiol functionality by nucleophilic substitution gives access to the desired polymers.

In a particular embodiment, a polymer (D) comprises the partial formula (V)

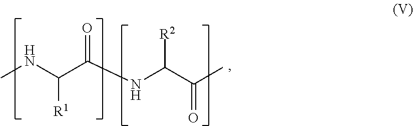

wherein

R¹ is a thiolalkyl-carbonyl substituent connected to said linker Z, wherein the Y-group of said linker Z is connected to the carbonyl-group of R¹,

R² is 2,3-dihydroxypropyl substituent,

and the relation between the two bracketed entities with R¹ and R², respectively, in the polymer indicates the relation of carbohydrate loading to capped thiol function.

For example, R¹ is of formula (Vn)

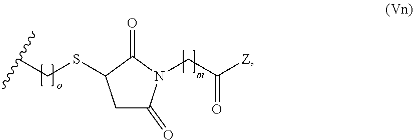

and R² is of formula (Vo)

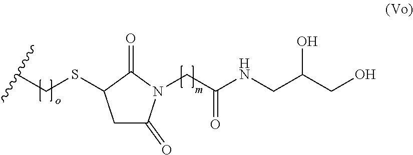

wherein o is between 1 and 6, preferably 1 or 2, in particular 1 and m is between 1 and 6, preferably between 1 and 2.

When o is 1, substituent R¹ represents a side chain of poly-cysteine, connected to said Y-group, preferably to said Y═NH₂ group, of said linker Z, which linker Z is comprised by the inventive compounds, and preferably by the inventive compounds of formula (I), (II), (III) or (IV), and R² is 2,3-dihydroxypropylthio-alkyl, i.e. a capped thiol function having a solubilizing substituent.

The poly-cysteine used to prepare polymer (D) of formula (V) has preferably a molecular weight between 1,000 and 300,000 Da, in particular 30,000 to 70,000 Da, and such polymers further connected via the Y-group of said linker Z to compounds of formula (I) and/or (II) and/or (III) and/or (IV) with a capping 2,3-dihydroxypropyl substituent residue are preferred. For example, linker Z, and hereby typically and preferably said Y═NH₂ group of said linker Z, is first functionalized by a peptide coupling reaction with an heterobifunctional maleimide-activated ester crosslinker. Reaction of the maleimide-functionalized linker Z with said polymer comprising the terminal thiol functionality by nucleophilic addition gives access to the desired polymers.

In a particular embodiment, a polymer (F) comprises the partial formula (VI)

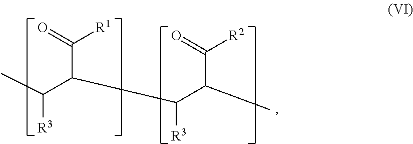

wherein

R¹ is said linker Z, wherein the Y is NH₂.

R² is 2,3-dihydroxypropylamino or a related amino substituent, and

R³ is hydrogen or methyl;

and the relation between the two bracketed entities with R¹ and R², respectively, in the polymer indicates the relation of carbohydrate loading to capped amide function.

For example, R¹ is Z

and R² is of formula (VIa),

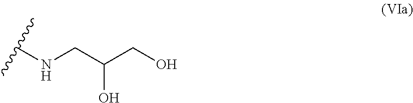 [SMILES: *NCC(O)CO]

The poly-acrylic acid used to prepare polymer (F) of formula (VI) has preferably a molecular weight between 1,000 and 400,000 Da, in particular 30,000 to 160,000 Da, and such polymers further connected via the NH-group of said linker Z to compounds of formula (I) and/or (II) and/or (III) and/or (IV) and with a capping 2,3-dihydroxypropylamino residue are preferred. For example, poly-acrylic acid is directly coupled to said linker Z comprising the terminal amine functionality by amide formation gives access to the desired polymers.

In a particular embodiment, a polymer (G) comprises the partial formula (VII)

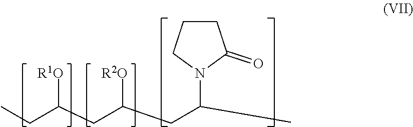

wherein

R¹ is a hydroxyalkyl or hydroxyaryl substituent connected to said linker Z, wherein the SH-group of said linker Z is connected to the —CH₂-group of R¹,

R² is 2,3-dihydroxypropylthioacetyl-hydroxyalkyl (or -hydroxyaryl),

and the relation between the two bracketed entities with R¹ and R², respectively, in the polymer indicates the relation of carbohydrate loading to capped hydroxy function.

For example, R¹ is of formula (VIIa)

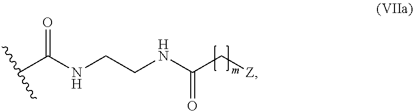

and R² is of formula (VIIIb)

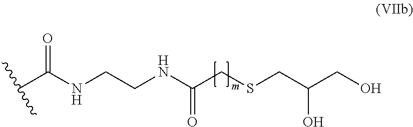

wherein m is between 1 and 10, preferably between 1 and 4.

The copolymer used to prepare polymer (G) of formula (VII) has preferably a molecular weight between 1,000 and 400,000 Da, in particular 30,000 to 160,000 Da, and such polymers further connected via the SH-group of said linker Z to compounds of formula (I) and/or (II) and/or (III) and/or (IV) and with a capping 2,3-dihydroxypropylthio-carbonylaminoalkylaminocarbonyl residue are preferred.

In a particular embodiment, a polymer (H) comprises the partial formula (VIII)

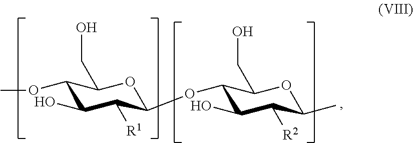

wherein

R¹ is an aminoalkyl substituent connected to said linker Z, wherein the Y-group of of said linker Z is connected to the terminal amino group of R¹ via a spacer moiety, wherein typically and preferably said spacer moiety is an acetyl group.

R² is 2,3-dihydroxypropylthioacetyl- acetylamine, which is a capped amino function having a solubilizing substituent,

and the relation between the two bracketed entities with R¹ and R², respectively, in the polymer indicates the relation of carbohydrate loading to capped amino function.

For example, R¹ is of formula (VIIIa)

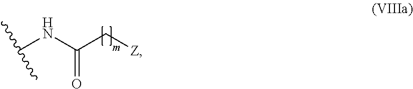

and R² is of formula (VIIIb)

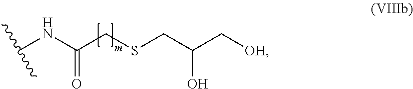

wherein m is between 1 and 6, preferably between 1 and 2, in particular 1.

The chitosan used to prepare polymer (H) of formula (VIII) has preferably a molecular weight between 1,000 and 300,000 Da, in particular 30,000 to 70,000 Da, and such polymers connected via the Y-group of said linker Z to compounds of formula (I) and/or (II) and/or (III) and/or (IV) and connected to a capping 2,3-dihydroxypropylthio-acetylamine residue are preferred. For example, the chitosan polymer is first functionalized by chloroacetylation of the amino groups. Reaction of the chloroacetylated polymer with said linker Z comprising the terminal thiol functionality by nucleophilic substitution gives access to the desired polymers.

In a particular embodiment, a polymer (I) comprises the partial formula (IX)

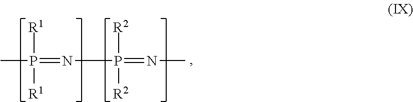

wherein

R¹ is a said linker Z. In the case wherein Y is NH₂, the phophorus group is connected to the amine-group of said linker Z.

R² is 2,3-dihydroxypropyl-amine,

and the relation between the two bracketed entities with R¹ and R², respectively, in the polymer indicates the relation of carbohydrate loading to capped carboxy function.

For example, R¹ is Z,

and R² is of formula (IXa)

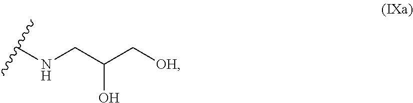 [SMILES: *NCC(O)CO]

The polyphosphazene used to prepare polymer (I) of formula (IX) has preferably a molecular weight between 1,000 and 300,000 Da, in particular 30,000 to 70,000 Da, and such polymers further connected via the Y-group of said linker Z to compounds of formula (I) and/or (II) and/or (III) and/or (IV) and with a capping 2,3-dihydroxypropylamine residue are preferred. For example, the polyphosphazene is first coupled by substitution to said linker Z comprising the terminal amino functionality gives access to the desired polymers.

From the group of polymers (A)-(I), preferred polymers are α-amino acid polymers (D- and L-form) or combinations (co-polymers) of different α-amino acids (A)-(D). More preferred are α-amino acid polymers consisting of poly-lysine, poly-ornithine, poly-aspartic acid, poly-glutamic acid, poly-glutamine, poly-asparagine. Particularly preferred among these α-amino acid polymers is poly-L-lysine.

In a further very preferred embodiment, said polymer is a polymer of formula 5, 6, 9, 23, 27, 32, 35, 38, 39 or 59, wherein said formulas are shown in the experimental section, and wherein for each of said polymer n is independently 20-1200, preferably 100-1100, further preferably 200-500, and wherein for each of said polymer x is independently 10-90, preferably 30-60, and further preferably 40-50.

In a further very preferred embodiment, said polymer is a polymer of formula 5, 6, 9, 23, 27, 32, 35, 38, 39 or 59, wherein said formulas are shown in the experimental section, and wherein for each of said polymer n is independently 100-1100, preferably 200-500, and wherein for each of said polymer x is independently 30-60, and further preferably 40-50.

In a further very preferred embodiment, said polymer is a polymer of formula 5, wherein said formula is shown in the experimental section, and wherein for said polymer n is 20-1200, preferably 100-1100, further preferably 200-500, and wherein for said polymer x is independently 10-90, preferably 15-60, and further preferably 20-40. In a further very preferred embodiment, said polymer is a polymer of formula 5, wherein said formula is shown in the experimental section, and wherein for said polymer n is 200-500, preferably 400, and wherein for said polymer x is independently 15-60, preferably 20-40, further preferably 25.

In a further very preferred embodiment, said polymer is a polymer of formula 6, wherein said formula is shown in the experimental section, and wherein for said polymer n is 20-1200, preferably 100-1100, further preferably 200-500, and wherein for said polymer x is independently 10-90, preferably 20-60, and further preferably 30-50. In a further very preferred embodiment, said polymer is a polymer of formula 6, wherein said formula is shown in the experimental section, and wherein for said polymer n is 200-500, preferably 400, and wherein for said polymer x is independently 15-60, preferably 20-40, further preferably 40.

In a further very preferred embodiment, said polymer is a polymer of formula 9, wherein said formula is shown in the experimental section, and wherein for said polymer n is 20-1200, preferably 100-1100, further preferably 200-500, and wherein for said polymer x is independently 10-90, preferably 20-80, and further preferably 30-75. In a further very preferred embodiment, said polymer is a polymer of formula 9, wherein said formula is shown in the experimental section, and wherein for said polymer n is 200-500, preferably 400, and wherein for said polymer x is independently 20-80, preferably 30-75, further preferably 68.

In a further very preferred embodiment, said polymer is a polymer of formula 23, wherein said formula is shown in the experimental section, and wherein for said polymer n is 20-1200, preferably 100-1100, further preferably 200-500, and wherein for said polymer x is independently 10-90, preferably 30-60, and further preferably 40-50. In a further very preferred embodiment, said polymer is a polymer of formula 23, wherein said formula is shown in the experimental section, and wherein for said polymer n is 200-500, preferably 400, and wherein for said polymer x is independently 30-60, preferably 40-50, further preferably 42.

In a further very preferred embodiment, said polymer is a polymer of formula 27, wherein said formula is shown in the experimental section, and wherein for said polymer n is 20-1200, preferably 100-1100, further preferably 200-500, and wherein for said polymer x is independently 10-90, preferably 15-60, and further preferably 20-40. In a further very preferred embodiment, said polymer is a polymer of formula 27, wherein said formula is shown in the experimental section, and wherein for said polymer n is 200-500, preferably 400, and wherein for said polymer x is independently 20-40, preferably 25.

In a further very preferred embodiment, said polymer is a polymer of formula 32, wherein said formula is shown in the experimental section, and wherein for said polymer n is 20-1200, preferably 100-1100, further preferably 200-500, and wherein for said polymer x is independently 10-90, preferably 15-60, and further preferably 20-40. In a further very preferred embodiment, said polymer is a polymer of formula 32, wherein said formula is shown in the experimental section, and wherein for said polymer n is 200-500, preferably 400, and wherein for said polymer x is independently 15-60, preferably 20-40 and further preferably 35.

In a further very preferred embodiment, said polymer is a polymer of formula 35, wherein said formula is shown in the experimental section, and wherein for said polymer n is 20-1200, preferably 100-1100, further preferably 200-500, and wherein for said polymer x is independently 10-90, preferably 15-60, and further preferably 20-40. In a further very preferred embodiment, said polymer is a polymer of formula 35, wherein said formula is shown in the experimental section, and wherein for said polymer n is 200-500, preferably 400, and wherein for said polymer x is independently 15-60, preferably 20-40 and further preferably 25.

In a further very preferred embodiment, said polymer is a polymer of formula 38, wherein said formula is shown in the experimental section, and wherein for said polymer n is 20-1200, preferably 100-1100, further preferably 200-500, and wherein for said polymer x is independently 10-90, preferably 15-60, and further preferably 20-40. In a further very preferred embodiment, said polymer is a polymer of formula 38, wherein said formula is shown in the experimental section, and wherein for said polymer n is 200-500, preferably 400, and wherein for said polymer x is independently 15-60, preferably 20-40, and further preferably 25.

In a further very preferred embodiment, said polymer is a polymer of formula 39, wherein said formula is shown in the experimental section, and wherein for said polymer n is 20-1200, preferably 100-1100, further preferably 200-500, and wherein for said polymer x is independently 10-90, preferably 15-60, and further preferably 20-40. In a further very preferred embodiment, said polymer is a polymer of formula 39, wherein said formula is shown in the experimental section, and wherein for said polymer n is 200-500, preferably 400, and wherein for said polymer x is independently 15-60, preferably 20-40, and further preferably 35.

In a further very preferred embodiment, said polymer is a polymer of formula 59, wherein said formula is shown in the experimental section, and wherein for said polymer n is 20-1200, preferably 100-1100, further preferably 200-500, and wherein for said polymer x is independently 10-90, preferably 15-60, and further preferably 20-40. In a further very preferred embodiment, said polymer is a polymer of formula 59, wherein said formula is shown in the experimental section, and wherein for said polymer n is 200-500, preferably 400, and wherein for said polymer x is independently 15-60, preferably 20-40, and further preferably 28.

The general terms used hereinbefore and hereinafter preferably have within the context of this disclosure the following meanings, unless otherwise indicated:

Where the plural form is used for compounds and the like, this is taken to mean also a single compound, or the like.

The term “glycoepitope”, as used herein, refers to the carbohydrate moiety that is recognized by a carbohydrate-binding protein (CBP). Preferably, the term “glycoepitope”, as used herein, refers to a carbohydrate moiety selected from glycolipids such as the globo- and ganglio-types; red blood cell glycoantigens; the Tn and sialyl-Tn antigens. Preferably, the term “glycoepitope”, as used herein, refers to the carbohydrate moiety that is recognized by a CBP, wherein said glycoepitope is comprised by compounds of formula (I) or formula (II) or formula (III) or formula (IV).

The term “reducing end”, as used herein in the context of the glycoepitope of the present invention and of the specific inventive compounds, refers to the terminal monosaccharide of the glycoepitope with a free anomeric carbon that is not involved in a glycosidic bond, wherein said free anomeric carbon bears a hemiacetal group.

The term “biodegradable”, as used herein, relates to metabolic biodegradability, cell- mediated biodegradablity, enzymatic biodegradability, hydrolytic biodegradability of the biodegradable polymeric backbone of the inventive polymer.

The term “C₁₋₄alkyl”, as used herein refers to straight or branched chain of 1 to 4 carbon atoms and includes butyl, such as n-butyl, sec-butyl, iso-butyl, tert-butyl, propyl, such as n-propyl or iso-propyl, ethyl or methyl. Preferably the term “C₁₋₄alkyl”, refers to methyl or ethyl, n-propyl or iso-propyl. Further preferably, the term “C₁₋₄alkyl”, refers to methyl. Correspondingly, the term “C₁₋₈alkyl”, as used herein refers to straight or branched chain of 1 to 8 carbon atoms. The term “C₁₋₄alkyl-(OCH₂CH₂)ₚO—C₁₋₄alkyl”, as used herein, and when referring to the linker Z defined as —X-A-(B)ₚ-(CH₂)_{q}—Y, and when referring to A within said linker Z, should refer, as evident from the description and examples herein, to a bivalent “C₁₋₄alkyl-(OCH₂CH₂)ₚO—C₁₋₄alkyl” group including groups such as —(CH₂)ₙ—(OCH₂CH₂)ₚO—(CH₂)ₙ— with n requal 1 to 4.

The term “C₁₋₇alkylene”, as used herein, refers to a straight or branched bivalent alkyl chain, preferably to a straight or branched bivalent alkyl chain of 1 to 7 carbon atoms, and includes, for example, —CH₂—, —CH₂—CH₂—, —CH(CH₃)—, —CH₂—CH₂—CH₂—, —CH(CH₃)—CH₂—, or —CH(CH₂CH₃)—.

The term “C₁₋₇alkoxy”, as used herein, refers to an alkoxy with a straight or branched chain of 1 to 7 carbon atoms. The term “C₁₋₄alkoxy”, as used herein, refers to an alkoxy with a straight or branched chain of 1 to 4 carbon atoms and includes methoxy, ethoxy, propoxy, iso-propoxy, n-butoxy, sec-butoxy and tert-butoxy. Preferably, the term “C₁₋₄alkoxy”, as used herein, refers to methoxy, ethoxy, propoxy. Further preferably, the term “C₁₋₄alkoxy”, as used herein, refers to methoxy. The term “C₁₋₇alkoxy”, as used herein, and when referring to the linker Z defined as —X-A-(B)ₚ-(CH₂)_{q}—Y, and when referring to said A within said linker Z, should refer, as evident from the description and examples herein, to a bivalent C₁-C₇-alkoxy group including groups such as —(CH₂)ₙO— or —O(CH₂)ₙ— with n requal 1 to 7, typically and very preferably to groups such as —O(CH₂)ₙ— forming with said X═N(R^{a}) of the linker Z a preferred bonding N(R^{a})—O(CH₂)ₙ—.

The term “C₁-C₈-alkenyl”, as used herein, refers to is a straight or branched chain containing one or more, e.g. two or three, double bonds, and is preferably Cᵢ₋₄alkenyl, such as 1- or 2-butenyl, 1-propenyl, allyl or vinyl.

Double bonds in principle can have E- or Z-configuration. The compounds of this invention may therefore exist as isomeric mixtures or single isomers. If not specified both isomeric forms are intended.

The term “C₁₋₈alkynyl”, as used herein, refers to a straight or branched chain comprising one or more, preferably one triple bond. Preferred are C₁-C₄-alkynyl, such as propargyl or acetylenyl.

Any asymmetric carbon atoms may be present in the (R)-, (S)- or (R,S)-configuration, preferably in the (R)- or (S)-configuration. The compounds may thus be present as mixtures of isomers or as pure isomers, preferably as enantiomer-pure diastereomers.

The term “aryl”, as used herein, refers to a mono- or bicyclic fused ring aromatic group with 5 to 10 carbon atoms optionally carrying substituents, such as phenyl, 1-naphthyl or 2-naphthyl, or also a partially saturated bicyclic fused ring comprising a phenyl group, such as indanyl, indolinyl, dihydro- or tetrahydronaphthyl, all optionally substituted. Preferably, aryl is phenyl, indanyl, indolinyl or tetrahydronaphthyl, in particular phenyl.

The term “heteroaryl”, as used herein, refers to an aromatic mono- or bicyclic ring system containing at least one heteroatom, and preferably up to three heteroatoms selected from nitrogen, oxygen and sulfur as ring members. Heteroaryl rings do not contain adjacent oxygen atoms, adjacent sulfur atoms, or adjacent oxygen and sulfur atoms within the ring. Monocyclic heteroaryl preferably refers to 5 or 6 membered heteroaryl groups and bicyclic heteroaryl preferably refers to 9 or 10 membered fused-ring heteroaryl groups. Examples of heteroaryl include pyrrolyl, thienyl, furyl, pyrazolyl, imidazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, and benzo or pyridazo fused derivatives of such monocyclic heteroaryl groups, such as indolyl, benzimidazolyl, benzofuryl, quinolinyl, isoquinolinyl, quinazolinyl, pyrrolopyridine, imidazopyridine, or purinyl, all optionally substituted.

Preferably, the term “heteroaryl” refers to a 5- or 6-membered aromatic monocyclic ring system containing at least one heteroatom, and preferably up to three heteroatoms selected from nitrogen, oxygen and sulfur as ring members. Preferably, heteroaryl is pyridyl, pyrimdinyl, pyrazinyl, pyridazinyl, thienyl, pyrazolyl, imidazolyl, thiazolyl, oxadiazolyl, triazolyl, oxazolyl, isoxazolyl, isothiazolyl, pyrrolyl, indolyl, pyrrolopyridine or imidazopyridine; in particular pyridyl, pyrimidinyl, pyrazinyl, pyridazinyl, pyrazolyl, imidazolyl, thiazolyl, oxadiazolyl, triazolyl, indolyl, pyrrolopyridine or imidazopyridine

The term “optionally substituted aryl”, as used herein, refers to aryl substituted by up to four substituents, preferably up to two substituents. In optionally substituted aryl, preferably in optionally substituted phenyl, substituents are preferably and independently selected from C₁-C₄-alkyl, C₁-C₄-alkoxy, amino-C₁-C₄-alkyl, acylamino-C₁-C₄-alkyl, aryl-C₁-C₄-alkyl hydroxy, carboxy, C₁-C₄-alkoxycarbonyl, aminocarbonyl, hydroxylaminocarbonyl, tetrazolyl, hydroxysulfonyl, aminosulfonyl, halo, or nitro, in particular C₁-C₄-alkyl, C₁- C₄-alkoxy, amino-C₁-C₄-alkyl, acylamino-C₁-C₄-alkyl, carboxy, C₁-C₄-alkoxycarbonyl, aminocarbonyl, hydroxylaminocarbonyl, tetrazolyl, or aminosulfonyl.

The term “optionally substituted heteroaryl”, as used herein, refers to heteroaryl substituted by up to three substituents, preferably up to two substituents. In optionally substituted heteroaryl, substituents are preferably and independently selected from C₁-C₄alkyl, C₁-C₄alkoxy, halo-C₁₋₄alkyl, hydroxy, C₁-C₄-alkoxycarbonyl, aminocarbonyl, hydroxylaminocarbonyl, tetrazolyl, aminosulfonyl, halo, aryl-C₁₋₄alkyl, or nitro.

Cycloalkyl has preferably 3 to 7 ring carbon atoms, and may be unsubstituted or substituted, e.g. by C₁₋₄alkyl or C₁₋₄alkoxy. Cycloalkyl is, for example, cyclohexyl, cyclopentyl, methylcyclopentyl, or cyclopropyl, in particular cyclopropyl.

Acyl designates, for example, alkylcarbonyl, cycloalkylcarbonyl, arylcarbonyl, aryl-C₁-C₄-alkylcarbonyl, or heteroarylcarbonyl. C₁₋₄acyl is preferably lower alkylcarbonyl, in particular propionyl or acetyl. Ac stands for acetyl.

Hydroxyalkyl is especially hydroxy-C₁-C₄a1kyl, preferably hydroxymethyl, 2-hydroxyethyl or 2-hydroxy-2-propyl.

Haloalkyl is preferably fluoroalkyl, especially trifluoromethyl, 3,3,3-trifluoroethyl or pentafluoroethyl.

Halogen is fluorine, chlorine, bromine, or iodine.

Arylalkyl includes aryl and alkyl as defined hereinbefore, and is e.g. benzyl, 1-phenethyl or 2-phenethyl.

Heteroarylalkyl includes heteroaryl and alkyl as defined hereinbefore, and is e.g. 2-, 3- or 4-pyridylmethyl, 1- or 2-pyrrolylmethyl, 1-pyrazolylmethyl, 1-imidazolylmethyl, 2-(1-imidazolyl)ethyl or 3-(1-imidazolyl)propyl.

In substituted amino, the substituents are preferably those mentioned as substituents hereinbefore. In particular, substituted amino is alkylamino, dialkylamino, optionally substituted arylamino, optionally substituted arylalkylamino, lower alkylcarbonylamino, benzoylamino, pyridylcarbonylamino, lower alkoxycarbonylamino or optionally substituted aminocarbonylamino.

Particular salts considered are those replacing the hydrogen atoms of carboxylic acid function. Suitable cations are, e.g., sodium, potassium, calcium, magnesium or ammonium cations, or also cations derived by protonation from primary, secondary or tertiary amines containing, for example, C₁-C₄-alkyl, hydroxy-C₁-C₄-alkyl or hydroxy-C₁-C₄-alkoxy-C₁-C₄-alkyl groups, e.g., 2-hydroxyethylammonium, 2-(2-hydroxyethoxy)ethyldimethylammonium, diethylammonium, di(2-hydroxyethyl)ammonium, trimethylammonium, triethylammonium, 2-hydroxyethyldimethylammonium, or di(2-hydroxyethyl)methylammonium, also from correspondingly substituted cyclic secondary and tertiary amines, e.g., N-methylpyrrolidinium, N-methylpiperidinium, N-methylmorpholinium, N-2-hydroethylpyrrolidinium, N-2-hydroxyethylpiperidinium, or N-2-hydroxyethylmorpholinium, and the like.

In view of the close relationship between the novel compounds in free form and those in the form of their salts, including those salts that can be used as intermediates, for example in the purification or identification of the novel compounds, any reference to the free compounds hereinbefore and hereinafter is to be understood as referring also to the corresponding salts, and vice versa, as appropriate and expedient

A preferred polymer backbone in the inventive polymers comprising a multitude of compounds of formula (I), formula (II), formula (III) or formula (IV) is polylysine, in particular poly-L-lysine.

Preferably the molecular weight of the polylysine is 1,000 to 300,000 Da, preferably 10,000 to 200,000 Da. Particularly preferred is a molecular weight of approximately 30,000 Da, 50,000 Da, 70,000 Da, 125,000 Da or 200,000 Da. Most preferred is a molecular weight of approximately 50,000 Da. Typically and preferably, the polylysine used in the present invention, and in particular the polylysine used for the described examples herein, has been purchased in the form of its hydrobromide salt. Typically and preferably, the preferred ranges of the molecular weight of the polylysine of the preferred embodiments of the present invention refers to the molecular weight of the polylysine and not its hydrobromide salt.

In particular the invention relates to such polymers wherein the relative loading of polymer backbone with the carbohydrate moiety of said compound of formula (I) and/or (II) and/or (III) and/or (IV) is 10-90%, meaning that 10-90% of all lysine side chains in the polymer are connected to said Y-group of said linker Z, which linker Z is comprised by the inventive compounds, and preferably by the inventive compounds of formula (I) or (II), or (III), or (IV), the remaining amino functions being capped. Preferably the loading of the polymer is 20-70%, more preferably 30-60%. Further preferred polymers in said context are polymers with loading of one or several of compounds of formula (I), (II), (III) or (IV), wherein said compounds of formula (I), (II), (III) or (IV), are selected from 3*, 8*, 22*, 26*, 31*, 34*, 37*, 45*, 47*-58*.

The polymers of the present invention comprising said multitude of compounds which compounds comprise carbohydrate moieties and linkers Z, wherein said carbohydrate moieties mimic glycoepitopes recognized by CBPs, allow straightforward coupling of said carbohydrate moieties to biodegradable poly-L-lysine and other functionalized biodegradable polymers. Thus, the resulting inventive chemically defined glycoconjugates/glycopolymers based on biodegradable polymer backbones can be used in a clinical context, either therapeutic and diagnostic, to detect or neutralize or remove pathogenic CBPs. Moreover, the multivalent presentation of the carbohydrate moieties mimicking glycoepitopes recognized by CBPs, on, preferably, poly-L-lysine, can substantially increase their binding affinity towards the CBPs.

In a particularly preferred embodiment, the invention relates to polymers comprising a multitude of compounds of formula (I), and/or (II), and/or (III), and/or (IV), wherein the polymer is poly-L-lysine and wherein said polymer further comprises said linker Z connecting said compounds to the polymer backbone via a spacer moeity. Poly-L-lysine is biodegradable and therefore in particular suitable for therapeutical application.

The polymers, compounds and compositions of the invention have valuable pharmacological properties. The invention also relates to polymers, compounds and compositions as defined hereinbefore for use as medicaments. A polymer, compound and composition according to the invention shows prophylactic and therapeutic efficacy especially against diseases associated with CBP-mediated cytotoxicity, agglutination or immune complex deposit formation.

One or multiple compounds of formula (I), and/or (II) and/or (III) and/or (IV) or polymers comprising these, can be administered alone or in combination with one or more other therapeutic agents, possible combination therapy taking the form of fixed combinations, or the administration of a polymer, compounds or composition of the invention and one or more other therapeutic agents being staggered or given independently of one another, or the combined administration of fixed combinations and one or more other therapeutic agents.

Therapeutic agents for possible combination are particularly antibiotics or immunosuppressive agents/ therapies. Examples for antibiotics are penicillins, cephalosporins, fluoroquinolones or aminoglycosides. Examples for immunosuppressive agents/therapies are purine analogues such as fludarabine and/or cladribine, plasmapheresis, intravenous immunoglobulins, furthermore anti-CD20⁺ antibodies such as rituximab.

In another particular embodiment, the invention relates to the use of the polymers, compounds and compositions of the invention in a diagnostic assay for diseases associated with CBP-mediated cytotoxicity, agglutination or immune complex deposit formation. In particular, the invention relates to kits comprising the compounds of formula (I), and/or (II), and/or (III), and/or (IV), as defined above, and also polymers of the invention comprising such compounds as substituents.

The present invention relates to a method of diagnosis of diseases associated with CBP-mediated cytotoxicity, agglutination or immune complex deposit formation, wherein the level of CBP is determined in a body fluid sample, e.g. serum, and a high level is indicative of the development and the severity of a particular disease.

Other body fluids than serum are useful for determination of CBPs and are, e.g., whole blood, cerebrospinal fluid or extracts from solid tissue.

Any known method may be used for the determination of the level of CBPs in body fluids. Methods considered are, e.g., ELISA, RIA, EIA, microarray, and glycanarray analysis.

A preferred method for the determination of CBPs, e.g. in serum, is an ELISA. In such an embodiment, microtiter plates are coated with compounds of formula (I), and/or (II), and/or (III), and/or (IV), or preferably polymers of the invention comprising such compounds as substituents. The plates are then blocked and the sample or a standard solution is loaded. After incubation, a CBP is applied, e.g. an CBP directly conjugated with a suitable label, e.g. with an enzyme for chromogenic detection. Alternatively, a polyclonal rabbit (or mouse) anti-CBP antibody is added. A second antibody detecting the particular type of CBP, e.g. an anti-rabbit (or anti-mouse) antibody, conjugated with a suitable label, e.g. the enzyme for chromogenic detection, is then added. Finally the plate is developed with a substrate for the label in order to detect and quantify the label, being a measure for the presence and amount of CBP. If the label is an enzyme for chromogenic detection, the substrate is a colour-generating substrate of the conjugated enzyme. The colour reaction is then detected in a microplate reader and compared to standards.

It is also possible to use antibody fragments. Suitable labels are chromogenic labels, i.e. enzymes which can be used to convert a substrate to a detectable colored or fluorescent or luminescent compound, spectroscopic labels, e.g. fluorescent or luminescent labels or labels presenting a visible color, affinity labels which may be developed by a further compound specific for the label and allowing easy detection and quantification, or any other label used in standard ELISA.

Other preferred methods of detection of CBPs are radioimmunoassay or competitive immunoassay and chemiluminescence detection on automated commercial analytical robots. Microparticle enhanced fluorescence, fluorescence polarized methodologies, or mass spectrometry may also be used. Detection devices, e.g. microarrays, glycanarrays, are useful components as readout systems for CBPs.

In a further embodiment the invention relates to a kit suitable for an assay as described above, in particular an ELISA, comprising compounds of formula (I), and/or (II) and/or (III) and/or (IV) or polymers comprising such compounds as substituents. The kits further contain CBPs (or CBP fragments) carrying a suitable label, or CBPs and second antibodies carrying such a suitable label, and reagents or equipment to detect the label, e.g. reagents reacting with enzymes used as labels and indicating the presence of such a label by a colour formation or fluorescence or luminescence, standard equipment, such as microtiter plates, pipettes and the like, standard solutions and wash solutions.

The ELISA can be also designed in a way that patient blood or serum samples are used for the coating of microtiter plates with the subsequent detection of CBPs with labelled compounds of formula (I), and/or (II), and/or (III), and/or (IV), or labelled polymers comprising such compounds as substituents. The label is either directly detectable or indirectly detectable via an antibody.

The polymer carrying compounds of formula (I), and/or (II), and/or (III), and/or (IV), of the invention typically and preferably binds to the pathogenic CBPs. It allows a targeted treatment for diseases associated with CBP-mediated cytotoxicity, agglutination or immune complex deposit formation.

Furthermore the invention relates to a pharmaceutical composition comprising a compound of formula (I), and/or (II), and/or (III), and/or (IV), or a polymer in accordance with the present invention carrying said compounds of formula (I), and/or (II), and/or (III), and/or (IV), of the invention.

Pharmaceutical compositions for parenteral administration, such as subcutaneous, intravenous, intrahepatic or intramuscular administration, to warm-blooded animals, especially humans, are considered. The compositions comprise the active ingredient(s) alone or, preferably, together with a pharmaceutically acceptable carrier. The dosage of the active ingredient(s) depends upon the age, weight, and individual condition of the patient, the individual pharmacokinetic data, and the mode of administration.

For parenteral administration preference is given to the use of suspensions or dispersions of the carbohydrate polymer of the invention, especially isotonic aqueous dispersions or suspensions which, for example, can be made up shortly before use. The pharmaceutical compositions may be sterilized and/or may comprise excipients, for example preservatives, stabilizers, wetting agents and/or emulsifiers, solubilizers, viscosity-increasing agents, salts for regulating osmotic pressure and/or buffers and are prepared in a manner known per se, for example by means of conventional dissolving and lyophilizing processes.

Suitable carriers for enteral administration, such as nasal, buccal, rectal or oral administration, are especially fillers, such as sugars, for example lactose, saccharose, mannitol or sorbitol, cellulose preparations, and/or calcium phosphates, for example tricalcium phosphate or calcium hydrogen phosphate, and also binders, such as starches, for example corn, wheat, rice or potato starch, methylcellulose, hydroxypropyl methylcellulose, sodium carboxymethylcellulose, and/or polyvinylpyrrolidone, and/or, if desired, disintegrators, such as the above-mentioned starches, also carboxymethyl starch, crosslinked polyvinylpyrrolidone, alginic acid or a salt thereof, such as sodium alginate. Additional excipients are especially flow conditioners and lubricants, for example silicic acid, talc, stearic acid or salts thereof, such as magnesium or calcium stearate, and/or polyethylene glycol, or derivatives thereof.

Tablet cores can be provided with suitable, optionally enteric, coatings through the use of, inter alia, concentrated sugar solutions which may comprise gum arabic, talc, polyvinyl-pyrrolidone, polyethylene glycol and/or titanium dioxide, or coating solutions in suitable organic solvents or solvent mixtures, or, for the preparation of enteric coatings, solutions of suitable cellulose preparations, such as acetylcellulose phthalate or hydroxypropyl-methylcellulose phthalate. Dyes or pigments may be added to the tablets or tablet coatings, for example for identification purposes or to indicate different doses of active ingredient(s).

Pharmaceutical compositions for oral administration also include hard capsules consisting of gelatin, and also soft, sealed capsules consisting of gelatin and a plasticizer, such as glycerol or sorbitol. The hard capsules may contain the active ingredient in the form of granules, for example in admixture with fillers, such as corn starch, binders, and/or glidants, such as talc or magnesium stearate, and optionally stabilizers. In soft capsules, the active ingredient is preferably dissolved or suspended in suitable liquid excipients, such as fatty oils, paraffin oil or liquid polyethylene glycols or fatty acid esters of ethylene or propylene glycol, to which stabilizers and detergents, for example of the polyoxyethylene sorbitan fatty acid ester type, may also be added.

Pharmaceutical compositions suitable for rectal administration are, for example, suppositories that consist of a combination of the active ingredient and a suppository base. Suitable suppository bases are, for example, natural or synthetic triglycerides, paraffin hydrocarbons, polyethylene glycols or higher alkanols.

The mentioned pharmaceutical compositions according to the invention may contain separate tablets, granules or other forms of orally acceptable formulation of the active ingredients, or may contain a mixture of active ingredients in one suitable pharmaceutical dosage form, as described above. In particular the separate orally acceptable formulations or the mixture in one suitable pharmaceutical dosage form may be slow release and controlled release pharmaceutical compositions.

The pharmaceutical compositions comprise from approximately 1% to approximately 95% active ingredient or mixture of active ingredients, single-dose administration forms comprising in the preferred embodiment from approximately 20% to approximately 90% active ingredient(s) and forms that are not of single-dose type comprising in the preferred embodiment from approximately 5% to approximately 20% active ingredient(s).

The invention also relates to the mentioned pharmaceutical compositions as medicaments in the treatment of diseases associated with CBP-mediated cytotoxicity, agglutination or immune complex deposit formation.

Thus, in a further aspect, the present invention provides for a polymer in accordance with the present invention, a compound in accordance with the present invention, or a pharmaceutical composition in accordance with the present invention for use in a method of treating a disease or disorder, wherein said disease or disorder is selected from a bacterial infection, an agglutination disorder or a disorder caused by immune complex deposits, wherein preferably said bacterial infection is caused by Shigella, preferably by S. dysenteriae, Escherichia coli, Vibrio cholerae, Clostridium difficile, Clostridium botulinum, Clostridium tetani, Bordetella pertussis; and wherein preferably said agglutination disorder is caused by anti-A agglutinins, anti-B agglutinins, anti-I system agglutinins, anti-P system agglutinins, or anti-Tn and anti-sialyl-Tn agluttinins; and wherein preferably said disorder caused by immune complex deposit-forming immunoglobulins is caused by immunoglobulins binding to the Tn and sialyl-Tn antigen on other immunoglobulins, preferably selected from IgG, IgA, IgM.

In a further preferred embodiment, said disease or disorder is a bacterial infection, wherein preferably said bacterial infection is caused by Shigella, and wherein said bacterial infection is preferably shigellosis, bacillary dysentery, Marlow syndrome or hemolytic-uremic syndrome (HUS).

In a further preferred embodiment, said disease or disorder is a bacterial infection, wherein preferably said bacterial infection is caused by Escherichia coli, and wherein said bacterial infection is preferably travelers' diarrhea.

In a further preferred embodiment, said disease or disorder is a bacterial infection, wherein preferably said bacterial infection is caused by Vibrio cholerae, and wherein said bacterial infection is preferably cholera.

In a further preferred embodiment, said disease or disorder is a bacterial infection, wherein preferably said bacterial infection is caused by Clostridium difficile, and wherein said bacterial infection is preferably Clostridium difficile infection.

In a further preferred embodiment, said disease or disorder is a bacterial infection, wherein preferably said bacterial infection is caused by Clostridium botulinum, and wherein said bacterial infection is preferably botulinism.

In a further preferred embodiment, said disease or disorder is a bacterial infection, wherein preferably said bacterial infection is caused by Clostridium tetani, and wherein said bacterial infection is preferably tetanus.

In a further preferred embodiment, said disease or disorder is a bacterial infection, wherein preferably said bacterial infection is caused by Bordetella pertussis, and wherein said bacterial infection is preferably pertussis or whooping cough.

In a further preferred embodiment, said disease or disorder is an agglutination disorder, wherein preferably said agglutination disorder is caused by anti-A agglutinins, and wherein preferably said agglutination disorder is ABH-incompatible transplantation/transfusion.

In a further preferred embodiment, said disease or disorder is an agglutination disorder, wherein preferably said agglutination disorder is caused by anti-B agglutinins, and wherein preferably said agglutination disorder is ABH-incompatible transplantation/transfusion.

In a further preferred embodiment, said disease or disorder is an agglutination disorder, wherein preferably said agglutination disorder is caused by anti-I system agglutinins, and wherein preferably said agglutination disorder is cold agluttinin disease.

In a further preferred embodiment, said disease or disorder is an agglutination disorder, wherein preferably said agglutination disorder is caused by anti-P system agglutinins, and wherein preferably said agglutination disorder is paroxysmal cold hemoglobinuria.

In a further preferred embodiment, said disease or disorder is an agglutination disorder, wherein preferably said agglutination disorder is caused by anti-Tn or anti-sialyl-Tn agluttinins, and wherein preferably said agglutination disorder is Tn polyagglutinability syndrome.

In a further preferred embodiment, said disease or disorder is a disorder caused by immune complex deposit-forming immunoglobulins, wherein preferably said disorder is IgA nephropathy (also known as IgA nephritis or Berger disease or synpharyngitic glomerulonephritis) or IgA vasculitis (also known as Henoch Schönlein Purpura (HSP).

In a further aspect, the present invention provides for a polymer in accordance with the present invention, a compound in accordance with the present invention, or a pharmaceutical composition in accordance with the present invention for use in a method of treating a disease or disorder, wherein said disease or disorder is selected from a bacterial infection, an agglutination disorder or a disorder caused by immune complex deposits, and wherein preferably said disease or disorder is selected from shigellosis, bacillary dysentery, Marlow syndrome, hemolytic-uremic syndrome (HUS), travelers' diarrhea, cholera, Clostridium difficile infection, botulinism, tetanus, pertussis or whooping cough, ABH-incompatible transplantation/transfusion, cold agluttinin disease, paroxysmal cold hemoglobinuria, Tn polyagglutinability syndrome, IgA nephropathy (also known as IgA nephritis or Berger disease or synpharyngitic glomerulonephritis) or IgA vasculitis (also known as Henoch Schönlein Purpura (HSP).

In a further aspect, the present invention provides for a polymer or use in a method of treating a disease or disorder, wherein said disease or disorder is selected from a bacterial infection, an agglutination disorder or a disorder caused by immune complex deposits, preferably a bacterial infection, and wherein preferably said disease or disorder is selected from shigellosis, bacillary dysentery, Marlow syndrome, hemolytic-uremic syndrome (HUS), travelers' diarrhea, cholera, Clostridium difficile infection, botulinism, tetanus, pertussis or whooping cough, ABH-incompatible transplantation/transfusion, cold agluttinin disease, paroxysmal cold hemoglobinuria, Tn polyagglutinability syndrome, IgA nephropathy (also known as IgA nephritis or Berger disease or synpharyngitic glomerulonephritis) or IgA vasculitis (also known as Henoch Schönlein Purpura (HSP); and wherein said polymer comprises a multitude of a compound, wherein said compound comprises a carbohydrate moiety and a linker Z, and wherein said carbohydrate moiety mimics a glycoepitope recognized by a carbohydrate-binding protein (CBP), wherein said CBP is selected from a bacterial exotoxin, an agluttinin and an immune complex deposit-forming immunoglobulin, and wherein said linker Z is —X-A-(B)ₚ-(CH₂)_{q}—Y, wherein
X is O or N(R^{a});
R^{a} is H, C₁₋₄-alkyl, C₁₋₄alkoxy, CH₂C₆H₅, CH₂CH₂C₆H₅, OCH₂C₆H₅, or OCH₂CH₂C₆H₅;
A is C₁₋₇alkylene, OC₁₋₇alkylene, C₁₋₄alkylene-(OCH₂CH₂)ᵣOC₁₋₄alkylene, OC₁₋₇alkylene-R^{b}, or R^{b}-C₁₋₇alkylene wherein R^{b} is an optionally substituted aryl or an optionally substituted heteroaryl, and wherein r is 0 to 6, preferably r is 1, 2 or 3, and further preferably r is 1;
B is NHC(O) or S;
p is 0 or 1;
q is 0 to 6, preferably q is 0 to 4, and further preferably q is 0, 2 or 3;
Y is SH, N₃ or NH₂; and
wherein said linker Z is covalently bound via its —X-group to the reducing end of said carbohydrate moiety; and wherein

said multitude of said compound is connected to the polymer backbone by way of said linker Z, and wherein said connection is effected via the Y-group of said linker Z.

In a further aspect, the present invention provides for a polymer in accordance with the present invention, a compound in accordance with the present invention, or a pharmaceutical composition in accordance with the present invention for use in a method of diagnosis of a disease or disorder, wherein said disease or disorder is selected from a bacterial infection, an agglutination disorder or a disorder caused by immune complex deposit-forming immunoglobulins, wherein preferably said disease or disorder is selected from shigellosis, bacillary dysentery, Marlow syndrome, hemolytic-uremic syndrome (HUS), travelers' diarrhea, cholera, Clostridium difficile infection, botulinism, tetanus, pertussis or whooping cough, ABH-incompatible transplantation/transfusion, cold agluttinin disease, paroxysmal cold hemoglobinuria, Tn polyagglutinability syndrome, IgA nephropathy (also known as IgA nephritis or Berger disease or synpharyngitic glomerulonephritis) or IgA vasculitis (also known as Henoch Schönlein Purpura (HSP).

In a further aspect, the present invention provides for a polymer in accordance with the present invention, a compound in accordance with the present invention, or a pharmaceutical composition in accordance with the present invention for use in a method of diagnosis of a disease or disorder, wherein said disease or disorder is selected from a bacterial infection, an agglutination disorder or a disorder caused by immune complex deposit-forming immunoglobulins, wherein preferably said bacterial infection is caused by Shigella, Escherichia coli, Vibrio cholerae, Clostridium difficile, Clostridium botulinum, Clostridium tetani, Bordetella pertussis; and wherein preferably said agglutination disorder is caused by anti-A agglutinins, anti-B agglutinins, anti-I/i system agglutinins, anti-P system agglutinins, or anti-Tn and anti-sialyl-Tn agluttinins; and wherein preferably said disorder caused by immune complex deposits is caused by immunoglobulins binding to the Tn and sialyl-Tn antigen on other immunoglobulins, typically and preferably on IgG, IgA, IgM).

The present invention, thus, relates furthermore to a method of treatment of treatment of a disease associated with CBP-mediated cytotoxicity, agglutination or immune complex deposit formation, which comprises administering a polymer or composition according to the invention in a quantity effective against said disease, to a warm-blooded animal requiring such treatment. The pharmaceutical compositions can be administered prophylactically or therapeutically, preferably in an amount effective against the said diseases, to a warm-blooded animal, for example a human, requiring such treatment. In the case of an individual having a bodyweight of about 70 kg the daily, weekly or monthly dose administered is from approximately 0.01 g to approximately 5 g, preferably from approximately 0.1 g to approximately 1.5 g, of the active ingredients in a composition of the present invention.

The following Examples serve to illustrate the invention without limiting the invention in its scope.

### EXAMPLES

General Methods

NMR spectra were obtained on a Bruker Avance DMX-500 (500 MHz) spectrometer. Assignment of ¹H and ¹³C NMR spectra was achieved using 2D methods (COSY, HSQC and HMBC). Chemical shifts are expressed in ppm using residual HDO as references. IR spectra were recorded using a Perkin-Elmer Spectrum One FT-IR spectrometer. Electron spray ionization mass spectra (ESI-MS) were obtained on a Waters micromass ZQ. HRMS analysis was carried using an Agilent 1100LC equipped with a photodiode array detector and a micromass QTOF I equipped with a 4 GHz digital-time converter. Reactions were monitored by ESI-MS and TLC using glass plates coated with silica gel 60 F₂₅₄ (Merck) and visualized by using UV light and/or by charring with mostain (a 0.02 M solution of ammonium cerium sulfate dihydrate and ammonium molybdate tetrahydrate in 10% aq H₂SO₄). Column chromatography was performed on silica gel (Redisep normal phase silica gel column 35/70) or RP-18 (Merck LiChroprep® RP-18 40/63). Dimethylformamide (DMF) was purchased from Acros (99.8%, extra dry, over molecular sieves). Molecular sieves (MS, 4 Å) were activated in vacuo at 400° C. for 30 min immediately before use. Size-exclusion chromatography was performed on polyacrylamide gel (Biogel P-2 Fine). Dialysis was performed on a Biotech Cellulose Ester (CE) Membrane (SpectrumLabs, molecular weight cutoff: 100-500 Da). Centrifugations were carried out with an Eppendorf Centrifuge 5804 R. rt=room temperature.

Nine glycopolymers were synthesized (5, 6, Scheme 1; 9, Scheme 2; 23, Scheme 4; 27, Scheme 5; 32, Scheme 7; 35, Scheme 8; 38, 39, Scheme 9; 59, Scheme 11) for biological evaluation. Polylysine glycoconjugates 5, 6 bear the same carbohydrate (Gb3) but different in the ligand loading. Polylysine glycoconjugate 9 bears the blood group A antigen type 5 tetrasaccharide. Polylysine glycoconjugate 23 bears a mimetic of the Gb3 trisaccharide. Polylysine glycoconjugate 27 bears the Tn antigen. The synthesis of the galactose acceptor 18 is described in Scheme 3. The synthesis of linker 2 is described in Scheme 6. Polylysine glycoconjugate 32 bears the blood group A trisaccharide antigen. Polylysine glycoconjugate 35 bears the blood group B trisaccharide antigen. Polylysine glycoconjugates 38, 39 bear the Tn-Thr antigen. Polylysine glycoconjugate 59 bears the GM1a antigen. The synthesis of thiol 45 is described in Scheme 10. All reagents were bought from Sigma Aldrich, Acros, Alfa-Aesar, Elicityl or Alamanda Polymers. Chloroacetylated poly-L-lysine 4 (400 lysine repeating units) was synthesized from commercial poly-L-lysine hydrobromide polymer (obtained from Alamanda Polymers, MW=84 kDa), according to a published procedure (G. Thoma et al., J Am Chem Soc 1999, 121, 5919-5929). Derivatives 12 and 19 were synthesized according to published procedures (respectively R. Bukowski, et al. Eur J Org Chem, 2001, 2697-2705; Z. Zhang, et al, J Am Chem Soc, 1999, 121, 734-753). Compound 30 and 33 were synthesized according to published procedure Geeta Karki, et al. Glycoconj J, 2016, 63-78. Compound 36 was synthesized according to published procedure Geert-Jan Boons et al. 2012, US 20120039984 A1. Compound 46 was synthesized according to published procedure Sun, B., et al. Sci. China Chem., 2012, 55, 31-35.

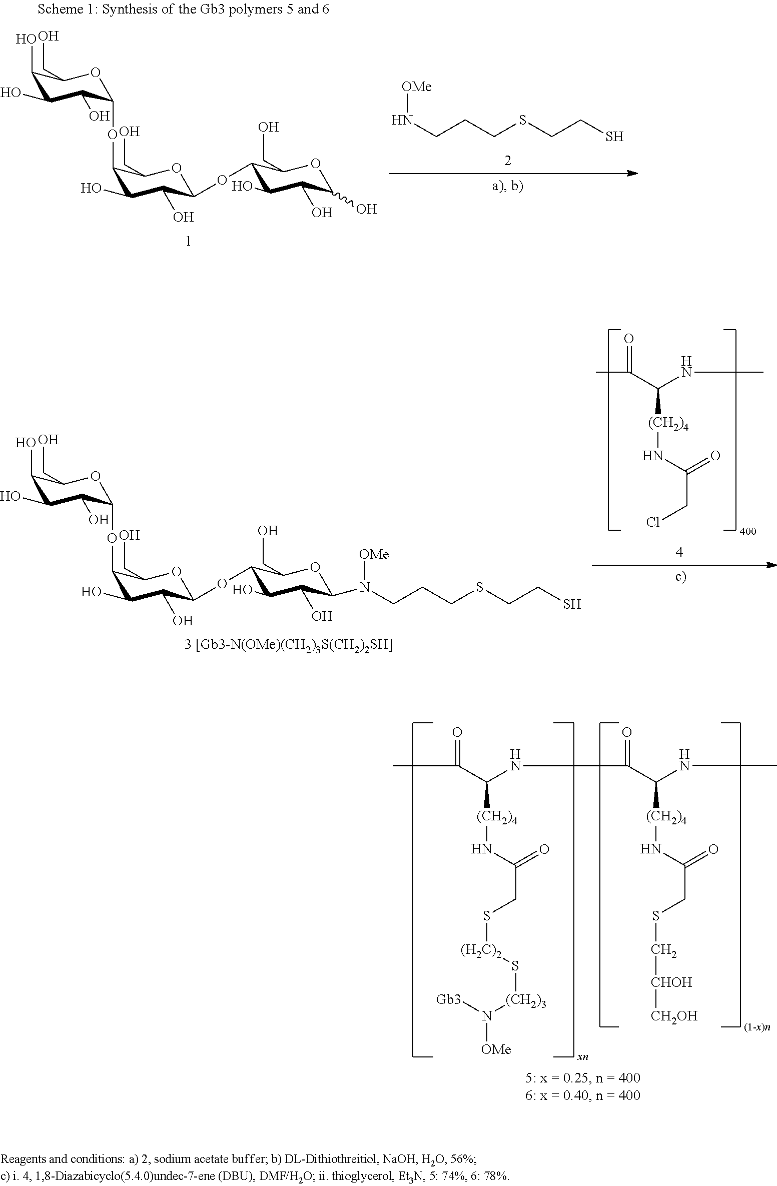

### N-(O-Methyl-N-[2-(2-ethylthio)propylthio]hydroxylamino)-α-D-galactopyranosyl-(1→4)-β-D-galactopyranosyl-(1→4)-β-D-glucopyranoside (3)

To a solution of hemiacetal 1 (78.0 mg, 155 μmol) in NaOAc/AcOH buffer (2.0 M, pH 4.5, 1.6 mL) was added oxyamine 2 (140 mg, 773 μmol, 5.0 equiv). The reaction mixture was stirred for 48 h at 40° C. After that time, the reaction mixture was concentrated. The crude residue was dissolved in water (2.0 mL). DL-Dithiothreitol (240 mg, 1.57 mmol, 10 equiv) was added followed by a few drops of a 1.0 M aq NaOH solution, until pH reached 9. The reaction mixture was stirred at rt under Ar for 3 h. Purification by reverse phase chromatography (0→100% MeOH in H₂O) gave compound 3 (58 mg, 86.9 μmol, 56%) as a white fluffy solid.

¹H-NMR (500 MHz, D₂O): δ 4.88 (d, J=3.9 Hz, 1H), 4.44 (d, J=7.8 Hz, 1H), 4.28 (t, J=6.5 Hz, 1H), 4.13 (d, J=8.8 Hz, 1H), 3.97 (d, J=3.9 Hz, 1H), 3.96 (d, J=3.9 Hz, 1H), 3.90 (m, 1H), 3.86 (m, 1H), 3.84 (dd, J=3.1 Hz, 1H), 3.80-3.74 (m, 3H), 3.72 (m, 1H), 3.67 (dd, J=10.3, 3.1 Hz, 1H), 3.65-3.62 (m, 2H), 3.60-3.54 (m, 6H), 3.51 (dd, J=6.8, 10.4 Hz, 1H), 3.45 (m, 1H), 3.10 (td, J=6.8, 13.2 Hz, 1H), 2.92 (m, 1H), 2.76-2.73 (m, 1H), 2.71-2.69 (m, 1H), 2.62 (t, J=7.3 Hz, 2H), 1.88-1.79 (m, 2H).

MS (ESI⁺): m/z 690.33 (calc. for C₂₄H₄₅NO₁₆S₂Na⁺ [M+Na]⁺ 690.21).

### Gb3 Polymer (5)

A solution of polymer 4 (5.3 mg, 26 μmol) in DMF (260 μL) was added to thiol 3 (3.5 mg, 5.2 μmol, 0.2 equiv). Water (13 μL) and a solution of DBU (2.3 μL, 16 μmol, 0.6 equiv) in DMF (21 μL) were successively added to the reaction mixture. After stirring for 90 min at rt, thioglycerol (6.7 μL, 78 μmol, 3.0 equiv) and Et₃N (10.8 μL, 78 μmol, 3.0 equiv) were added. The reaction mixture was stirred at rt for another 16 h. The product was precipitated by slow addition to a stirring solution of EtOH/Et₂O (1:1, 4 mL). The precipitate was filtered off, washed with EtOH and dried. Further purification was achieved by ultrafiltration (Sartorius Stedim Vivaspin tubes, 6 mL, molecular weight cutoff 10 kDa, 5000 rpm). Freeze-drying gave the Gb3 polymer 5 (7.86 mg, 74%) as a white solid. According to ¹H NMR, the product contained approximately 25% of the lysine side-chains substituted by the carbohydrate epitope 3.

### Gb3 Polymer (6)

A solution of polymer 4 (4.0 mg, 20 μmol) in DMF (195 μL) was added to thiol 3 (5.2 mg, 7.8 μmol, 0.4 equiv). Water (10 μL) and a solution of DBU (3.5 μL, 24 μmol, 1.2 equiv) in DMF (32 μL) were successively added to the reaction mixture. After stirring for 90 min at rt, thioglycerol (5.1 μL, 59 μmol, 3.0 equiv) and Et₃N (8.2 μL, 59 μmol, 3.0 equiv) were added. The reaction mixture was stirred at rt for another 16 h. The product was precipitated by slow addition to a stirring solution of EtOH/Et₂O (1:1, 4 mL). The precipitate was filtered off, washed with EtOH and dried. Further purification was achieved by ultrafiltration (Sartorius Stedim Vivaspin tubes, 6 mL, molecular weight cutoff 10 kDa, 5000 rpm). Freeze-drying gave the Gb3 polymer 6 (7.50 mg, 78%) as a white solid. According to ¹H NMR, the product contained approximately 40% of the lysine side-chains substituted by the carbohydrate epitope 3.

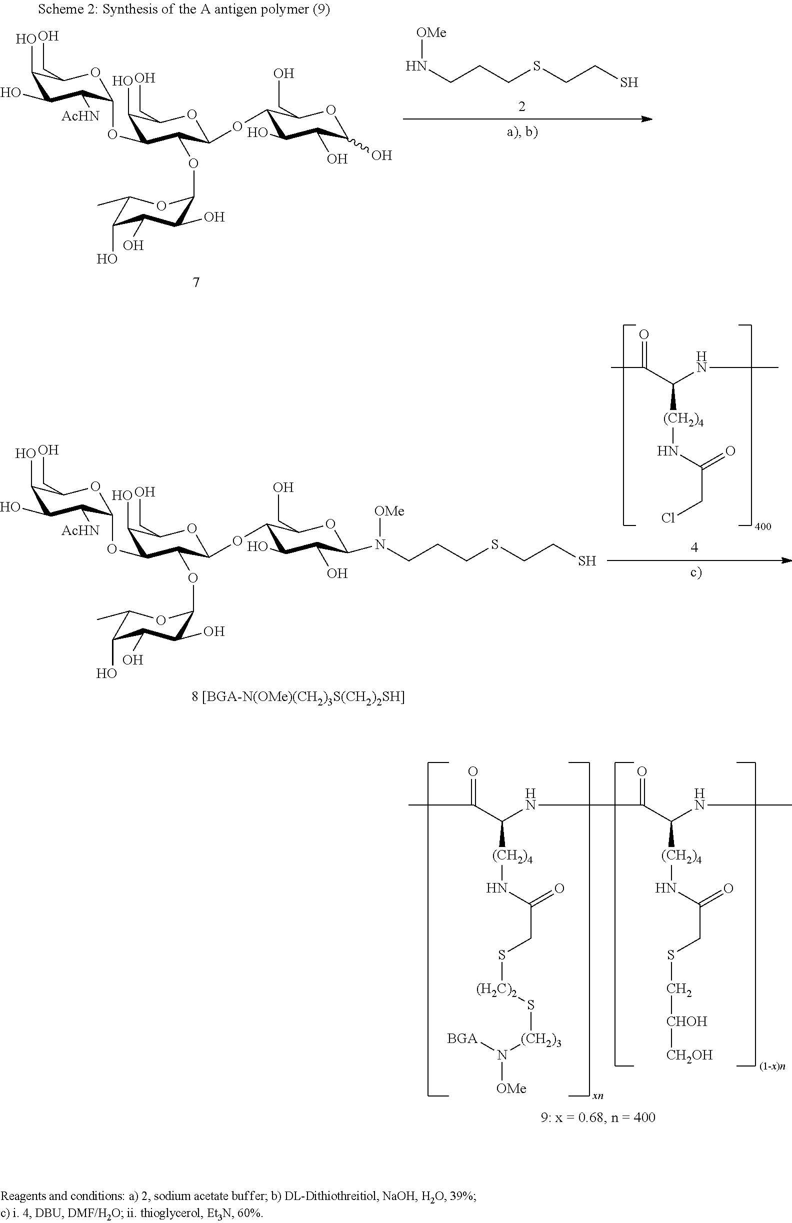

### N-(O-Methyl-N-[2-(2-ethylthio)propylthio]hydroxylamino)-α-D-galactopyranosyl-(1→3)-[α-L-fucopyranosyl-(1→2)]-β-D-galactopyranosyl-(1→4)-β-D-glucopyranoside (8)

To a solution of hemiacetal 7 (10.3 mg, 14.9 μmol) in NaOAc/AcOH buffer (2.0 M, pH 4.5, 75 μL) was added oxyamine 2 (14 mg, 75 μmol, 5.0 equiv). The reaction mixture was stirred for 40 h at 40° C. After that time, the reaction mixture was concentrated. The crude residue was dissolved in water (0.3 mL). DL-Dithiothreitol (30 mg, 0.19 mmol, 13 equiv) was added followed by a few drops of a 1.0 M aq NaOH solution, until pH reached 9. The reaction mixture was stirred at rt under Ar for 2 h. Purification by reverse phase chromatography (0→100% MeOH in H₂O) gave compound 3 (5.0 mg, 5.85 μmol, 39%) as a white fluffy solid.

¹H-NMR (500 MHz, D₂O): δ 5.31 (d, J=4.1 Hz, 1H), 5.14 (d, J=3.8 Hz, 1H), 4.55 (d, J=7.6 Hz, 1H), 4.29 (q, J=6.6 Hz, 1H), 4.21-4.16 (m, 3H), 4.12 (d, J=8.7 Hz, 1H), 3.96-3.91 (m, 3H), 3.89-3.84 (m, 2H), 3.80 (m, 1H), 3.78-3.75 (m, 1H), 3.75-3.69 (m, 5H), 3.68 (m, 1H), 3.65 (m, 1H), 3.62 (m, 1H), 3.59 (s, 3H), 3.58 (m, 1H), 3.56 (m, 1H), 3.34 (ddd, J=9.6, 5.7, 1.5 Hz, 1H), 3.14 (td, J=12.6, 7.2 Hz, 1H), 2.96 (m, 2H), 2.94 (m, 2H), 2.22 (m, 2H), 2.76-2.62 (m, 2H), 2.00 (s, 3H), 1.86 (dq, J=14.4, 7.2 Hz, 2H), 1.21 (d, J=6.6 Hz, 3H).

MS (ESI⁺): m/z 877.46 (calc. for C₃₂H₅₈N₂O₂₀S₂Na⁺ [M+Na]⁺ 877.29).

### A Antigen Polymer (9)

A solution of polymer 4 (1.8 mg, 8.6 μmol) in DMF (86 μL) was added to thiol 8 (3.4 mg, 4.3 μmol, 0.2 equiv). Water (4.0 μL) and a solution of DBU (1.3 μL, 9.6 μmol, 0.6 equiv) in DMF (12 μL) were successively added to the reaction mixture. After stirring for 1 h at rt, thioglycerol (2.2 μL, 26 μmol, 3.0 equiv) and Et₃N (3.6 μL, 26 μmol, 3.0 equiv) were added. The reaction mixture was stirred at rt for another 16 h. The product was precipitated by slow addition to a stirring solution of EtOH/Et₂O (1:1, 2 mL). The precipitate was filtered off, washed with EtOH and dried. Further purification was achieved by ultrafiltration (Sartorius Stedim Vivaspin tubes, 6 mL, molecular weight cutoff 10 kDa, 5000 rpm). Freeze-drying gave the blood group A antigen polymer 9 (4.07 mg, 60%) as a white solid. According to ¹H NMR, the product contained approximately 68% of the lysine side-chains substituted by the carbohydrate epitope 8.

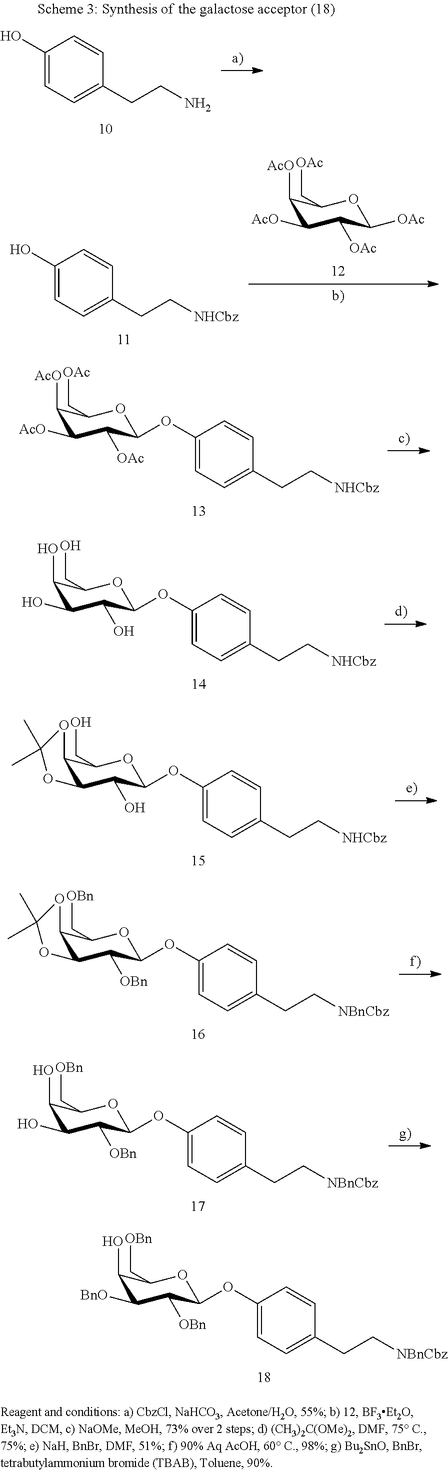

### Benzyl 4-hydroxyphenethylcarbamate (11)

A mixture of tyramine 10 (25.3 g, 185 mmol), acetone (200 mL), H₂O (100 mL), and satd aq NaHCO₃ was cooled to 0° C. and stirred under argon. CbzCl (26.0 mL, 183 mmol) was added portionwise during 20 min. The reaction was allowed to slowly warmed to rt and stirred for 2 d at rt. After that time, the acetone was evaporated and some yellow precipitate formed. The mixture was extracted with DCM (300 mL). The DCM phase was washed subsequently with 1 M aq H₂SO₄ (50 mL) and satd aq NaHCO₃ (50 mL), dried over Na₂SO₄, filtered, and concentrated in vacuo to give an off-white solid. The solid was recrystallized from EtOAc/n-heptane to give derivative 11 (27.8 g, 103 mmol, 55%).

¹H NMR (500 MHz, CDCl₃): δ 7.33 (m, 5H), 7.02 (d, J=8.0 Hz, 2H), 6.75 (d, J=8.3 Hz, 2H), 5.23 (s, 1H), 5.09 (s, 2H), 4.76 (s, 1H), 3.42 (q, J=6.5 Hz, 2H), 2.74 (t, J=6.9 Hz, 2H).

### 4-(2-((Benzyloxycarbonyl)amino)ethyl)phenyl β-D-galactopyranoside (14)

A mixture of donor 12 (16.6 g, 42.6 mmol), acceptor 11 (10.5 g, 38.7 mmol) and NEt₃ (3.2 mL) in dry DCM (40 mL) was cooled to 0° C. BF₃.Et₂O (8.6 mL, 69.7 mmol) was added dropwise. The solution was warmed to rt and stirred for 1 d. After that time, additional BF₃.Et₂O (8.6 mL, 69.7 mmol) was added dropwise and the solution was stirred for additional 12 h until completion was reached. The reaction mixture was poured into a mixture of ice-water (100 mL) and DCM (200 mL). The DCM phase was separated, washed with satd aq NaHCO₃ (50 mL), dried over Na₂SO₄, filtered, and concentrated in vacuo to give intermediate 13 as an oil. The oil was dissolved in MeOH (300 mL) and treated with NaOMe (36.1 mmol in 20 mL MeOH) overnight at rt. Water (100 mL) was added and the pH was adjusted to −5 with 1 N aq HCl. The solution was concentrated to ˜300 mL and additional water (100 mL) was added. Then the remaining MeOH was evaporated and a precipitate formed. The precipitate was filtered, washed with water (200 mL) and dried in vacuo (20 mbar) at rt for 1 h. The solid was recrystallized from propan-2-ol/EtOAc/n-heptane to give alcohol 14 (8.95 g, 25.1 mmol, 73%) as off-white needles.

¹H NMR (500 MHz, CDCl₃/CD₃OD): δ 7.34 (t, J=7.1 Hz, 5H), 7.10 (d, J=8.0 Hz, 2H), 7.01 (d, J=8.3 Hz, 2H), 5.08 (s, 2H), 4.83 (d, J=7.7 Hz, 1H), 3.97 (d, J=3.1 Hz, 1H), 3.81 (m, 3H), 3.64 (t, J=5.8 Hz, 1H), 3.60 (dd, J=3.3, 9.6 Hz, 1H), 3.37 (m, 2H), 2.76 (t, J=6.8 Hz, 2H).

MS (ESI⁺): m/z 456.08 (calcd for C₂₂H₂₇NNaO₈⁺ [M+Na]⁺: 456.16).

### 4-(2-((Benzyloxycarbonyl)amino)ethyl)phenyl 3,4-O-isopropylidene-β-D-galactopyranoside (15)

To a solution of tetraol 14 (4.90 g, 11.3 mmol) in DMF (20 mL) were added 2,2-dimethoxypropane (4.20 mL, 34.3 mmol) and p-TsOH.H₂O (41 mg) under argon. After stirring overnight at 75° C., water (20 mL) was added and the mixture was heated to 90° C. for 1 h. The reaction mixture was neutralized with NEt₃ (0.5 mL), concentrated under reduced pressure and co-evaporated with xylene (30 mL) to remove DMF and water. The residue was purified by flash chromatography on silica (petrol ether/EtOAc+10% MeOH, gradient 20-80%) to yield acetal 12 (4.01 g, 8.48 mmol, 75%) as an oil.

¹H NMR (500 MHz, CDCl₃): δ 7.31 (m, 5H), 7.10 (d, J=8.3 Hz, 2H), 6.92 (d, J=8.4 Hz, 2H), 4.99 (s, 2H), 4.79 (d, J=8.0 Hz, 1H), 4.17 (dd, J=1.4, 5.5 Hz, 1H), 4.05 (t, J=6.6 Hz, 1H), 3.97 (t, J=5.7 Hz, 1H), 3.58 (dd, J=5.3, 11.1 Hz, 1H), 3.53 (dd, J=7.3, 11.1 Hz, 1H), 3.46 (t, J=7.5 Hz, 1H), 3.18 (t, J=7.0 Hz, 2H), 2.65 (t, J=7.2 Hz, 2H), 1.42, 1.27 (2 s, 6H).

### 4-(2-(Benzyl(benzyloxycarbonyl)amino)ethyl)phenyl 2,6-di-O-benzyl-3,4-O-isopropylidene-β-D-galactopyranoside (16)

To a solution of diol 15 (3.00 g, 6.34 mmol) in DMF (12 mL) was added NaH (60% in mineral oil, 1.34 g, 33.4 mmol) at 0° C. and the mixture was stirred at rt for 30 min. After cooling to 0° C., benzyl bromide (3.0 mL, 25.3 mmol) was added and the reaction was stirred at 0° C. to rt overnight. Then the reaction mixture was quenched with diethylamine (10 mL), the excess of diethylamine was evaporated in vacuo below 30° C. and the remains were poured into cooled 1 N aq HCl (200 mL). The mixture was extracted with EtOAc (2×100 mL). The combined organic phases were washed with satd aq NaHCO₃ (100 mL), dried over Na₂SO₄, filtered, and then concentrated in vacuo. The residue was purified by flash chromatography on silica (petrol ether/acetone, gradient 10-80%) to yield fully protected derivative 16 (2.38 g, 3.20 mmol, 51%) as an oil.

¹H NMR (500 MHz, CDCl₃, mixture of rotamers): δ 7.41-6.93 (m, 14H), 5.19 (bs, 1.6H), 4.88 (m, 3H), 4.61 (d, J=11.8 Hz, 1H), 4.53 (d, J=11.8 Hz, 1H), 4.42-4.37 (m, 2H), 4.25 (t, J=6.1 Hz, 1H), 4.21 (dd, J=1.8, 5.6 Hz, 1H), 4.04 (m, 1H), 3.83 (dd, J=4.9, 10.3 Hz, 1H), 3.78 (m, 1H), 3.66 (t, J=7.2 Hz, 1H), 3.44-3.37 (m, 2H), 2.74 (m, 2H), 1.41, 1.35 (2 s, 6H).

MS (ESI⁺): m/z 766.35 (calcd for C₄₆H₄₉NNaO₈⁺ [M+Na]⁺: 766.34).

### 4-(2-(Benzyl(benzyloxycarbonyl)amino)ethyl)phenyl 2,6-di-O-benzyl-β-D-galactopyranoside (17)

A solution of acetal 16 (2.24 g, 3.02 mmol) in 90% aq AcOH (20 ml) was stirred at 70° C. overnight. The solvents were evaporated and the residue was purified by flash chromatography on silica (DCM/MeOH, 1:0 to 9:1) to yield diol 17 (2.08 g, 2.96 mmol, 98%) as an oil.

¹H NMR (500 MHz, CDCl₃, mixture of rotamers): δ 7.21 (m, 24H), 5.18 (bs, 2H), 5.03 (d, J=11.4 Hz, 1H), 4.93 (d, J=7.6 Hz, 1H), 4.77 (d, J=11.4 Hz, 1H), 4.56 (s, 2H), 4.41 (m, 2H), 4.05 (t, J=3.0 Hz, 1H), 3.82 (dd, J=5.2, 9.9 Hz, 1H), 3.77 (m, 3H), 3.67 (dt, 1H, J=3.8, 9.3 Hz), 3.41 (m, 2H), 2.75 (m, 2H), 2.64 (d, J=3.1 Hz, 1H), 2.54 (d, J=4.3 Hz, 1H).

MS (ESI⁺): m/z 726.37 (calcd for C₄₃H₄₅NNaO₈⁺ [M+Na]⁺: 726.30).

### 4-(2-(Benzyl(benzyloxycarbonyl)amino)ethyl)phenyl 2,3,6-tri-O-benzyl-β-D-galactopyranoside (18)

Bu₂SnO (210 mg, 0.85 mmol, 1.2 equiv) was added to a solution of diol 17 (500 mg, 0.71 mmol) in anhyd Tol (11 mL). The mixture was stirred for 5 h at reflux using a ‘Dean-Stark’ apparatus. After cooling to rt, dry tetrabutylammonium bromide (TBAB, 115 mg, 0.36 mmol, 0.5 equiv), and benzyl bromide (118 μL, 0.99 mmol, 1.4 equiv) were added. The re-action mixture was stirred for 3 h at reflux. The temperature was lowered to rt and the reaction quenched with MeOH. After stirring at rt for 20 min, the volatiles were evaporated under reduced pressure. The residue was purified by flash chromatography (Tol/EtOAc 9:1 to 85:15) to give acceptor 18 (537 mg, 90%) as a white solid.

¹H NMR (500 MHz, CDCl₃, mixture of rotamers): δ 7.42-7.19 (m, 24H), 5.18 (s, 2H), 4.98 (d, J=10.9 Hz, 1H), 4.91 (d, J=7.4 Hz, 1H, H-1), 4.82 (d, J=11.0 Hz, 1H), 4.74 (s, 2H), 4.56 (s, 2H), 4.43, 4.37 (2 s, 2H), 4.07 (t, 1H), 3.92 (t, J=8.6 Hz, 1H), 3.82 (dd, J=5.6, 9.9 Hz, 1H), 3.75 (dd, J=6.4, 9.6 Hz, 1H), 3.68 (t, 1H), 3.58 (dd, J=3.3, 9.3 Hz, 1H), 3.44, 3.37 (2 s, 2H), 2.79, 2.70 (2 s, 2H).

MS (ESI⁺): m/z 816.28 (calcd for C₅₀H₅₁NNaO₈⁺ [M+Na]⁺: 816.35).

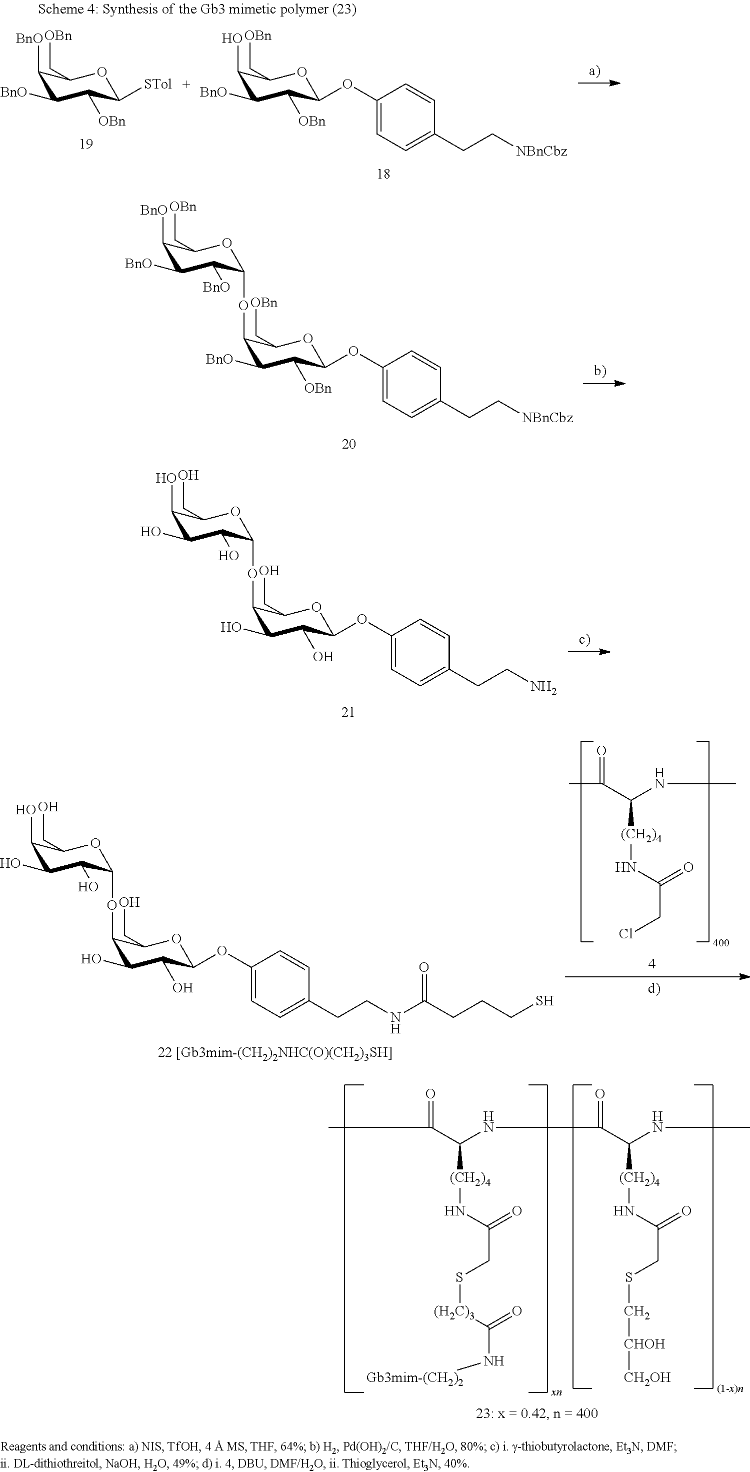

### 4-(2-(Benzyl(benzyloxycarbonyl)amino)ethyl)phenyl 2,3,4,6-tetra-O-benzyl-α-D-galactopyranosyl-(1→4)-2,3,6-tri-O-benzyl-β-D-galactopyranoside (20)

To a solution of acceptor 18 (100 mg, 0.13 mmol) and donor 19 (90 mg, 0.14 mmol, 1.1 equiv) in anhyd THF (2.6 mL) was added previously activated 4 Å MS and the suspension was stirred at rt for 30 min under Ar. After that time, NIS (57 mg, 0.25 mmol, 2.0 equiv) was added and the reaction mixture was cooled to −78° C., followed by addition of TfOH (1.1 μL, 0.01 mmol, 0.1 equiv). The reaction mixture was stirred for 1 h at −78° C. The reaction mixture was neutralized with Et₃N and the suspension was filtered over Celite. The filtrated was diluted with EtOAc, washed with satd aq Na₂S₂O₃ and brine and dried over anhyd Na₂SO₄. The suspension was filtrated and concentrated under reduced pressure. Purification by flash chromatography eluting with toluene/EtOAc (1:0→95:5) yielded the disaccharide 20 (109 mg, 0.083 mmol, 64%) as a colourless oil.

¹H-NMR (500 MHz, CDCl₃) δ 7.39-7.12 (m, 49H), 5.18 (s, 2H), 5.04 (s, 1H), 4.95 (d, J=10.6 Hz, 1H), 4.92 (d, J=11.6 Hz, 1H), 4.90 (d, J=8.2 Hz, 1H), 4.88 (d, J=12.0 Hz, 1H), 4.84 (d, J=11.0 Hz), 4.81 (d, J=12.4 Hz, 1H), 4.77 (s, 2H), 4.67 (d, J=11.8 Hz, 1H), 4.56 (d, J=11.6 Hz, 1H), 4.45 (dd, J=5.0, 9.1 Hz, 1H), 4.42, 4.36 (2 s, 2H), 4.20, 4.17 (2 d, J=2.3 Hz, 4H), 4.11 (m, 3H), 4.06 (d, J=2.7 Hz, 1H), 3.95 (m, 2H), 3.61-3.58 (m, 2H), 3.56 (t, J=8.8 Hz, 1H), 3.46 (dd, J=2.6, 10.0 Hz, 1H), 3.43, 3.36 (2 s, 2H), 3.27 (dd, J=4.8, 8.4 Hz, 1H), 2.78, 2.69 (2 s, 2H).

MS (ESI⁺): m/z 1338.69 (calcd for C₈₄H₈₅NNaO₁₃⁺ [M+Na]⁺: 1338.59).

### 4-(2-(amino)ethyl)phenyl α-D-galactopyranosyl-(1→4)-β-D-galactopyranoside (21)

To a solution of derivative 20 (57 mg, 43 μmol) in THF/AcOH (10:1, 5.5 mL) was added Pd(OH)₂/C (37 mg). The reaction mixture was stirred under an H₂ atmosphere for 17 h. After that time, additional Pd(OH)₂/C (25 mg) was added to the reaction mixture. After stirring for another 6 h under an Ar atmosphere, the suspension was filtered over a PTFE Acrodisc 0.45 μm membrane. The membrane was washed with 1-2 mL of THF. Additional Pd(OH)₂/C (30 mg) was added to the filtrate and the suspension was stirred over an H₂ atmosphere for another 17 h. After that time, the reaction mixture was filtered over a PTFE Acrodisc 0.45 μm membrane and concentrated under reduced pressure. The crude amine 21 (16 mg, 35 μmol, 80%) was used directly in the next step without further purification.

¹H NMR (500 MHz, D₂O) δ 7.24 (d, J=8.7 Hz, 2H), 7.08 (d, J=8.7 Hz, 2H), 5.09-5.06 (m, 1H), 4.93 (d, J=3.9 Hz, 1H), 4.33 (t, J=6.5 Hz, 1H), 4.04 (s, 1H), 3.98 (d, J=2.5 Hz, 1H), 3.88-3.84 (m, 3H), 3.82-3.76 (m, 5H), 3.66 (dd, J=6.4, 1.5 Hz, 2H), 3.19 (t, J=7.3 Hz, 2H), 2.90 (t, J=7.2 Hz, 2H).

MS (ESI⁺): m/z 462.21 (calcd for C₂₀H₃₂NO₁₁⁺ [M+H]⁺: 462.20).

### 4-(2-(4-mercaptobutanamido)ethyl)phenyl α-D-galactopyranosyl-(1→4)-β-D-galactopyranoside (22):

To a suspension of amine 21 (15 mg, 29 μmop in anhyd DMF (1.0 μL) were successively added γ-thiobutyrolactone (25 μL, 288 μmol, 10 equiv) and Et₃N (40 μL, 288 μmol, 10 equiv). The reaction mixture was stirred for 48 h at 40° C. After that time, the reaction mixture was concentrated. The crude residue was dissolved in water (0.5 mL). DL-Dithiothreitol (89 mg, 575 μmol, 20 equiv) was added followed by a few drops of a 1.0 M aq NaOH solution, until pH reached 9. The reaction mixture was stirred at rt under Ar for 3 h. After that time, the reaction mixture was neutralized with 20% aq AcOH, diluted with water and washed with EtOAc. The aqueous phase was concentrated in vacuo. Purification by reverse phase chromatography (0→100% MeOH in H₂O) gave compound 22 (8 mg, 14 μmol, 49%) as a white fluffy solid.

¹H NMR (500 MHz, D₂O) δ 7.19-7.15 (m, 2H), 7.05-7.01 (m, 2H), 5.04 (d, J=7.6 Hz, 1H), 4.93 (d, J=4.0 Hz, 1H), 4.33 (dt, J=1.0, 6.6 Hz, 1H), 4.04 (d, J=1.4 Hz 1H), 3.98 (dd, J=0.7, 2.5 Hz, 1H), 3.87 (dd, J=3.2, 10.5 Hz, 1H), 3.85-3.75 (m, 6H), 3.66-3.64 (m, 2H), 3.39 (t, J=6.6 Hz, 2H), 2.72 (t, J=6.6 Hz, 2H), 2.25 (t, J=7.1 Hz, 2H), 2.18 (t, J=7.2 Hz, 2H), 1.70-1.64 (m, 2H).

MS (ESI⁺): m/z 586.11 (calcd for C₂₄H₃₇NNaO₁₂S⁺ [M+Na]⁺: 586.19).

### Gb3 Mimetic Polymer (23)

A solution of polymer 4 (7.5 mg, 37 μmol) in DMF (365 μL) was added to thiol 22 (8.3 mg, 15 μmol, 0.4 equiv). Water (19 μL) and a solution of DBU (5.4 μL, 37 ∥mol, 1.0 equiv) in DMF (10 μL) were successively added to the reaction mixture. After stirring for 60 min at rt, thioglycerol (9.5 μL, 110 μmol, 3.0 equiv) and Et₃N (6.1 μL, 44 μmol, 1.2 equiv) were added. The reaction mixture was stirred at rt for another 17 h. The product was precipitated by slow addition to a stirring solution of EtOH/Et₂O (1:1, 4 mL). The precipitate was filtered off, washed with EtOH and dried. Further purification was achieved by ultrafiltration (Sartorius Stedim Vivaspin tubes, 6 mL, molecular weight cutoff 50 kDa, 5000 rpm). Freeze-drying gave the Gb3 mimetic polymer 23 (6.7 mg, 40%) as a white solid. According to ¹H NMR, the product contained approximately 42% of the lysine side-chains substituted by the carbohydrate epitope 22.

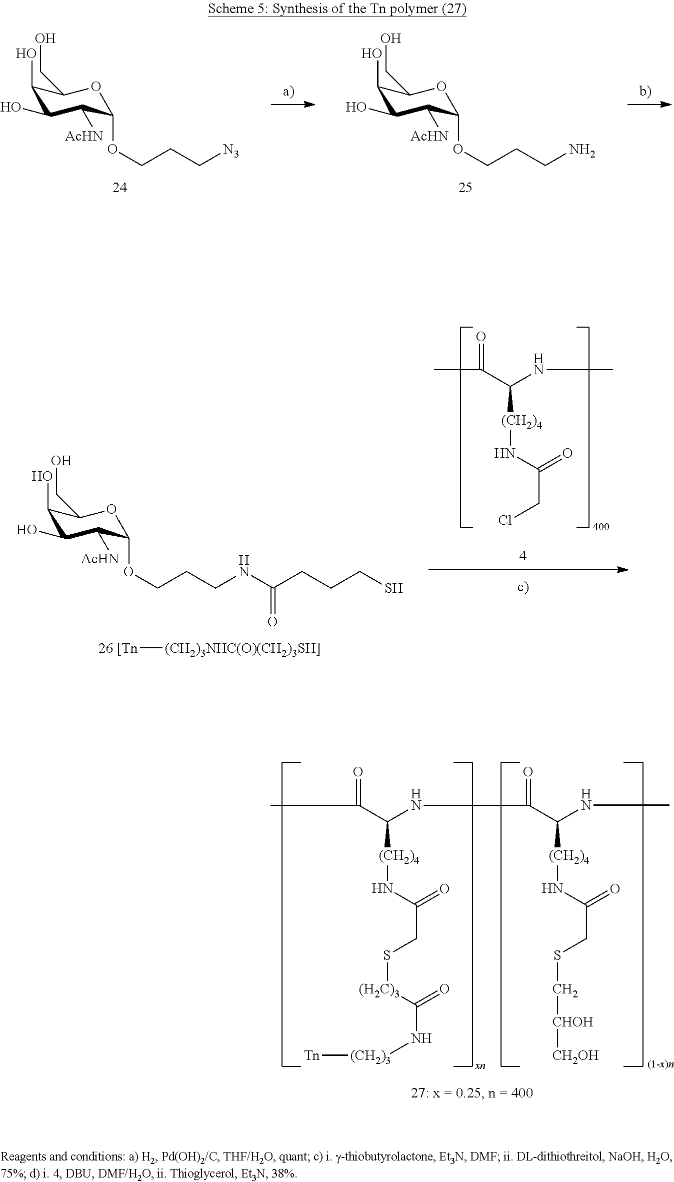

### 3-Aminopropyl 2-acetamido-α-D-galactopyranoside (25)

To a solution of azide 24 (28 mg, 9.2 μmol) in 20% aq AcOH (5.0 mL) was added Pd(OH)₂/C (30 mg). The reaction mixture was stirred under an H₂ atmosphere for 3 h. After that time, the reaction mixture was filtered over a PTFE Acrodisc 0.45 μm membrane and concentrated under reduced pressure. The crude amine 21 (isolated as the acetate salt, 26 mg, 9.2 μmol, quant) was used directly in the next step without further purification.

¹H NMR (500 MHz, D₂O) δ 4.92 (d, J=3.8 Hz, 1H), 4.18 (dd, J=3.8, 11.1 Hz, 1H), 4.00 (d, J=2.9 Hz, 1H), 3.96-3.93 (m, 1H), 3.93 (dd, J=3.2, 11.2 Hz, 1H), 3.85-3.80 (m, 1H), 3.77 (m, 2H), 3.61-3.55 (m, 1H), 3.13 (t, J=7.6 Hz, 2H), 2.06 (s, 3H), 2.04-1.99 (m, 2H), 1.98 (s, 3H).

MS (ESI⁺): m/z 279.12 (calcd for C₁₁H₂₃N₂O₆⁺ [M+Na]⁺: 279.16).

### 3-(4-mercaptobutanamido)propyl 2-acetamido-α-D-galactopyranoside (26)

To a suspension of amine 25 (25 mg, 74 μmol) in anhyd DMF (1.0 μL) were successively added γ-thiobutyrolactone (63 μL, 739 μmol, 10 equiv) and Et₃N (10 μL, 739 μmol, 10 equiv). The reaction mixture was stirred for 48 h at 40° C. After that time, the reaction mixture was concentrated. The crude residue was dissolved in water (0.5 mL). DL-Dithiothreitol (225 mg, 1.46 mmol, 20 equiv) was added followed by a few drops of a 1.0 M aq NaOH solution, until pH reached 9. The reaction mixture was stirred at rt under Ar for 3 h. Purification by reverse phase chromatography (0→100% MeOH in H₂O) followed by P2 size-exclusion chromatography gave compound 26 (21 mg, 55 μmol, 75%) as a white fluffy solid.

¹H NMR (500 MHz, MeOD) δ 4.79 (d, J=3.6 Hz, 1H), 4.26 (dd, J=11.0, 3.6 Hz, 1H), 3.88 (d, J=2.2 Hz, 1H), 3.85-3.66 (m, 5H), 3.41 (m, 1H), 3.34-3.27 (m, 2H), 2.52 (t, J=7.1 Hz, 2H), 2.31 (t, J=7.4 Hz, 2H), 2.01 (s, 3H), 1.88 (dq, J=14.3, 7.2 Hz, 2H), 1.81-1.77 (m, 2H).

MS (ESI⁺): m/z 403.13 (calcd for C₁₅H₂₈N₂NaO₇S⁺ [M+Na]⁺: 403.15).

### Tn Antigen Polymer (27)

A solution of polymer 4 (7.5 mg, 37 μmol) in DMF (365 μL) was added to thiol 26 (5.6 mg, 15 μmol, 0.4 equiv). Water (19 μL) and a solution of DBU (5.5 μL, 37 μmol, 1.0 equiv) in DMF (10 μL) were successively added to the reaction mixture. After stirring for 60 min at rt, thioglycerol (9.5 μL, 110 μmol, 3.0 equiv) and Et₃N (6.1 μL, 44 μmol, 1.2 equiv) were added. The reaction mixture was stirred at rt for another 17 h. The product was precipitated by slow addition to a stirring solution of EtOH/Et₂O (1:1, 2 mL). The precipitate was filtered off, washed with EtOH and dried. Further purification was achieved by ultrafiltration (Sartorius Stedim Vivaspin tubes, 6 mL, molecular weight cutoff 50 kDa, 5000 rpm). Freeze-drying gave the Tn antigen polymer 27 (4.8 mg, 38%) as a white solid. According to ¹H NMR, the product contained approximately 25% of the lysine side-chains substituted by the carbohydrate epitope 26.

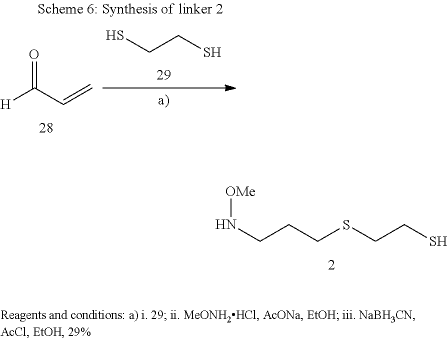

### 3-(3-(Methoxyamino)propylthio)propane-1-thiol (2)

Acrolein 28 (0.20 mL, 3.0 mmol) was added dropwise to 1,2-ethanedithiol 29 (1.3 mL, 15.0 mmol, 5.0 equiv) and the reaction mixture was stirred for 3 h at rt. After that time, the reaction mixture was diluted with EtOH (5.0 mL) and methoxyamine hydrochloride (300 mg, 3.6 mmol, 1.2 equiv) and NaOAc (492 mg, 6.0 mmol, 2.0 equiv) were added and the reaction mixture was stirred overnight at rt. After that time, NaBH₃CN (282 mg, 4.5 mmol, 1.5 equiv) was added to the reaction mixture, followed by dropwise addition of 1.0 M ethanolic HCl (10 mL, freshly prepared from AcCl and EtOH). After stirring for 1 h at rt, the reaction was neutralized by addition of satd aq NaHCO₃. The reaction mixture was diluted with H₂O and extracted with DCM (3×). The organic phases were pooled, washed with brine and dried over anhyd Na₂SO₄. The suspension was filtrated and concentrated under reduced pressure. Purification by flash chromatography eluting with Tol/Acetone (8:2) yielded the aminoalcohol 2 (159 mg, 0.88 mmol, 29%) as a colourless oil.

¹H-NMR (500 MHz, CDCl₃): δ 5.60 (s, 1H), 3.53 (s, 3H), 3.01 (t, 2H), 2.76 (m, 2H), 2.73 (m, 2H), 2.62 (t, 2H), 1.82 (m, 2H), 1.72 (dd, 1H).

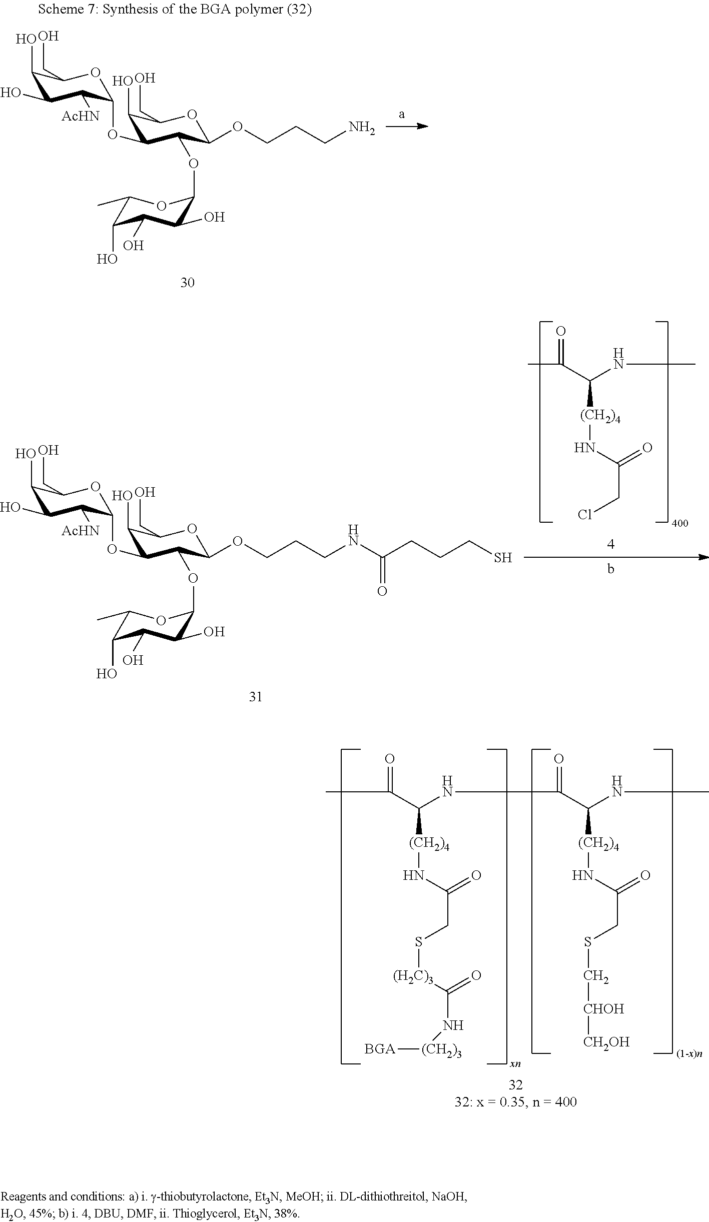

### 3-(4-mercaptobutanamido)propyl O-(2-acetamido-2-deoxy-α-D-galactopyranosyl)-(I→3)-[O-(α-L-fucopyranosyl)-(I→2)]-β-D-galactopyranoside (31)

To a suspension of amine 30 (20 mg, 0.03 μmol) in anhyd MeOH (2.0 μL) were successively added γ-thiobutyrolactone (35 μL, 0.34 μmol, 10 equiv) and Et₃N (34 μL, 0.34 μmol, 10 equiv). The reaction mixture was stirred for 24 h at 40° C. After that time, the reaction mixture was concentrated. The crude residue was dissolved in water (0.5 mL). DL-Dithiothreitol (105 mg, 20 equiv) was added followed by a few drops of a 1.0 M aq NaOH solution, until pH reached 9. The reaction mixture was stirred at rt under Ar for 3 h. Purification by reverse phase chromatography (0→100% MeOH in H₂O) followed by P2 size-exclusion chromatography gave compound 31 (12 mg, 54%) as a white fluffy solid.

¹H NMR (500 MHz, D₂O): 5.28 (d, J=3.7 Hz, 1H), 5.17 (d, J=3.7 Hz, 1H), 4.58 (d, J=7.7 Hz, 1H), 4.39-4.34 (m, 1H), 4.28-4.21 (m, 3H), 4.13-3.73 (m, 7H), 3.72-3.54 (m, 7H), 3.34-3.21 (m, 2H), 2.59-2.51 (m, 2H), 2.38 (t, J=7.2 Hz, 2H), 2.05 (s, 3H), 2.02-1.92 (m, 2H), 1.24 (d, J=6.5 Hz, 3H).

MS (ESI⁺): m/z 688.74 (calcd for C₂₇H₄₈N₂NaO₁₆S⁺ [M+Na]⁺: 711.26).

### BGA Antigen Polymer (32)

A solution of polymer 4 (2 mg, 9.8 μmol150) in DMF (150 μL) was added to thiol 31 (3.4 mg, 4.9 μmol) dissolved in 80 μL DMF. A solution of DBU (1.5 μL, 9.8 μmol) in DMF (10 μL) were successively added to the reaction mixture. After stirring for 60 min at rt, thioglycerol (2.5 μL, 29.3 μmol) and Et₃N (4.1 μL, 29.3 μmol) were added. The reaction mixture was stirred at rt for another 17 h. The product was precipitated by slow addition to a stirring solution of EtOH/Et₂O (1:1, 2 mL). The precipitate was filtered off, washed with EtOH and dried. Further purification was achieved by ultrafiltration (Sartorius Stedim Vivaspin tubes, 6 mL, molecular weight cutoff 50 kDa, 5000 rpm). Freeze-drying gave the BGA antigen polymer 32 (4.5 mg, 38%) as a white solid. According to ¹H NMR, the product contained approximately 35% of the lysine side-chains substituted by the carbohydrate epitope 31.

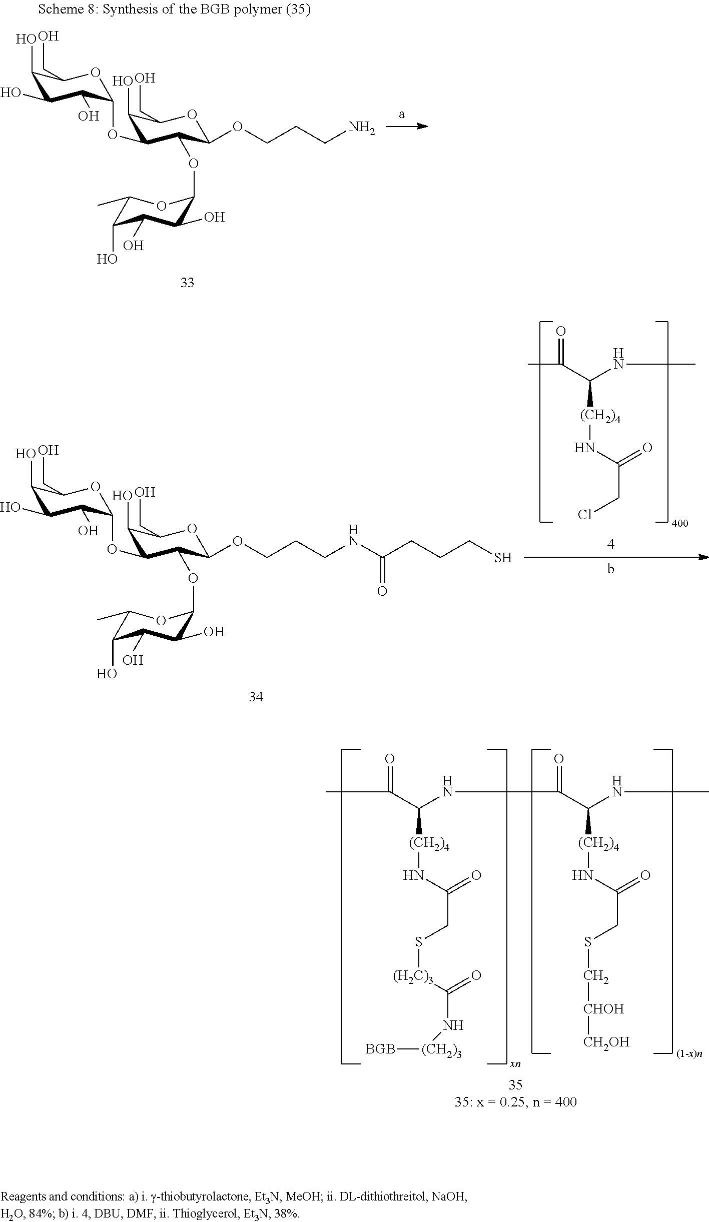

### 3-(4-mercaptobutanamido)propyl O-(α-D-galactopyranosyl)-(I→3)-[O-(α-L-fucopyranosyl)-(I→2)]-β-D-galactopyranoside (34)

To a suspension of amine 33 (12 mg, 0.02 μmol) in anhyd MeOH (2.0 μL) were successively added γ-thiobutyrolactone (19 μL, 0.22 □mol, 10 equiv) and Et₃N (31 μL, 0.22 μmol, 10 equiv). The reaction mixture was stirred for 24 h at 40° C. After that time, the reaction mixture was concentrated. The crude residue was dissolved in water (0.5 mL). DL-Dithiothreitol (68 mg, 20 equiv) was added followed by a few drops of a 1.0 M aq NaOH solution, until pH reached 9. The reaction mixture was stirred at rt under Ar for 3 h. Purification by reverse phase chromatography (0→100% MeOH in H₂O) followed by P2 size-exclusion chromatography gave compound 34 (12 mg, 84.2%) as a white fluffy solid.

¹H NMR (500 MHz, D₂O): 5.27 (d, J=3.5 Hz, 1H), 5.25 (d, J=3.3 Hz, 1H), 4.59 (d, J=7.3 Hz, 1H), 4.38-4.36 (m, 1H), 4.23 (m, 2H), 4.02-3.97 (m, 3H), 3.93-3.86 (m, 4H), 3.85-3.78 (m, 6H), 3.76-3.70 (m, 3H), 3.17-3.14 (m, 2H), 2.57-2.51 (m, 2H), 2.38 (t, J=7.2 Hz, 2H), 2.03-1.99 (m, 2H), 1.22 (d, J=6.5 Hz, 3H).

MS (ESI⁺): m/z 647.69 (calcd for C₂₅H₄₅NNaO₁₆S⁺ [M+Na]⁺: 670.24).

### BGB Antigen Polymer (35)

A solution of polymer 4 (3.0 mg, 14.7 μmol) in DMF (150 μL) was added to thiol 34 (3.8 mg, 5.9 μmol, 0.4 equiv). A solution of DBU (2.2 μL, 14.7 μmol, 1.0 equiv) in DMF (10 μL) were successively added to the reaction mixture. After stirring for 60 min at rt, thioglycerol (3.8 μL, 44 μmol, 3.0 equiv) and Et₃N (6.1 μL, 44 μmol, 3 equiv) were added. The reaction mixture as stirred at rt for another 17 h. The product was precipitated by slow addition to a stirring solution of EtOH/Et₂O (1:1, 2 mL). The precipitate was filtered off, washed with EtOH and dried. Further purification was achieved by ultrafiltration (Sartorius Stedim Vivaspin tubes, 6 mL, molecular weight cutoff 50 kDa, 5000 rpm). Freeze-drying gave the BGB antigen polymer 35 (4.5 mg, 41%) as a white solid. According to ¹H NMR, the product contained approximately 25% of the lysine side-chains substituted by the carbohydrate epitope 33.

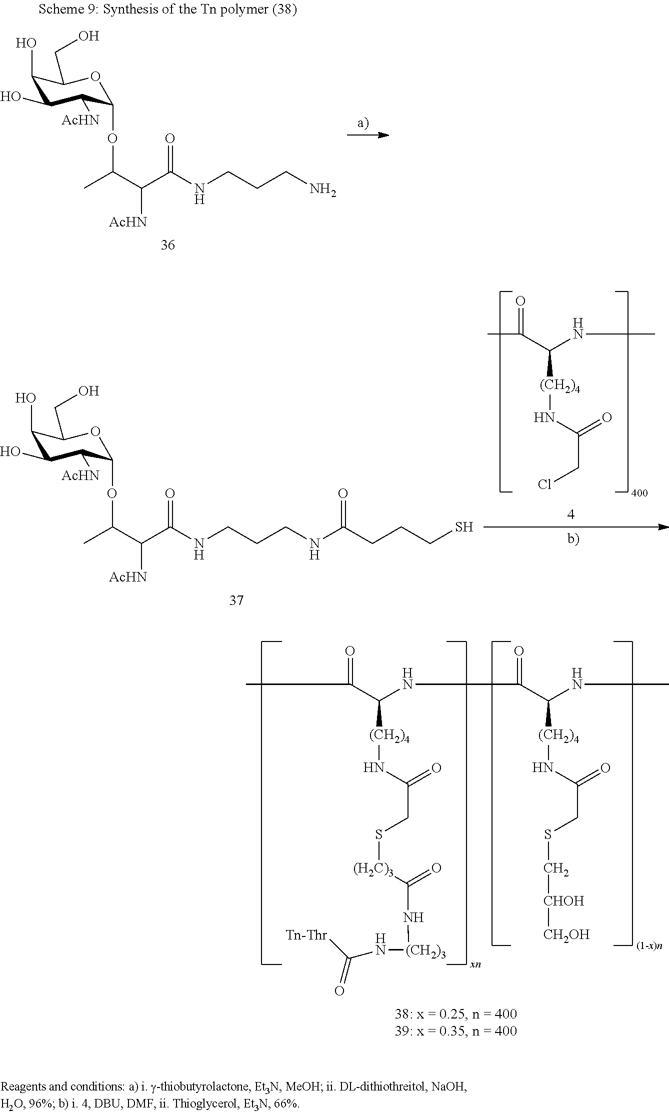

### Synthesis of Tn Thiol (37)

To a suspension of amine 36 (10 mg, 0.02 μmol) in anhyd MeOH (2.0 μL) were successively added γ-thiobutyrolactone (21 μL, 0.24 μl, 10 equiv) and Et₃N (33 μL, 0.24 μmol, 10 equiv). The reaction mixture was stirred for 24 h at 40° C. After that time, the reaction mixture was concentrated. The crude residue was dissolved in water (0.5 mL). DL-Dithiothreitol (73 mg, 20 equiv) was added followed by a few drops of a 1.0 M aq NaOH solution, until pH reached 9. The reaction mixture was stirred at rt under Ar for 3 h. Purification by reverse phase chromatography (0→100% MeOH in H₂O) followed by P2 size-exclusion chromatography gave compound 37 (11.6 mg, 96%) as a white fluffy solid.

¹H NMR (500 MHz, MeOD): 4.92 (d, J=3.6 Hz, 1H), 4.47-4.40 (m, 2H), 4.12 (dd, J=11.0, 3.8 Hz, 1H), 4.05 (t, J=6.1 Hz, 1H), 4.01 (d, J=2.9 Hz, 1H), 3.92 (dd, J=11.1, 3.1 Hz, 1H), 3.77 (t, J=6.8Hz, 2H), 3.43-3.34 (m, 3H), 3.26 (m, 1H), 2.80-2.72 (m, 2H), 2.55-2.49 (m, 1H), 2.40-2.32 (m, 2H), 2.18 (s, 3H), 2.08 (s, 3H), 1.93-1.86 (m, 1H), 1.74-1.67(m, 2H), 1.29 (d, J=6.3Hz, 3H).

MS (ESI⁺): m/z 538.61 (calcd for C₂₀H₃₈N₄NaO₁₀S⁺ [M+Na]⁺: 561.22).

### Tn Antigen Polymer (38)

A solution of polymer 4 (3.0 mg, 14.7 μmol) in DMF (150 μL) was added to thiol 37 (2.7 mg, 5.1 μmol, 0.35 equiv). A solution of DBU (2.2 μL, 14.7 μmol, 1.0 equiv) in DMF (10 μL) were successively added to the reaction mixture. After stirring for 60 min at rt, thioglycerol (3.8 μL, 44 μmol, 3.0 equiv) and Et₃N (6.1 μL, 44 μmol, 3 equiv) were added. The reaction mixture was stirred at rt for another 17 h. The product was precipitated by slow addition to a stirring solution of EtOH/Et₂O (1:1, 2 mL). The precipitate was filtered off, washed with EtOH and dried. Further purification was achieved by ultrafiltration (Sartorius Stedim Vivaspin tubes, 6 mL, molecular weight cutoff 50 kDa, 5000 rpm). Freeze-drying gave the Tn antigen polymer 38 (4.0 mg, 66%) as a white solid. According to ¹H NMR, the product contained approximately 25% of the lysine side-chains substituted by the carbohydrate epitope 37.

### Tn Antigen Polymer (39)

A solution of polymer 4 (3.0 mg, 14.7 μmol) in DMF (150 μL) was added to thiol 37 (3.8 mg, 7.3 μmol, 0.5 equiv). A solution of DBU (2.2 μL, 14.7 μmol, 1.0 equiv) in DMF (10 μL) were successively added to the reaction mixture. After stirring for 60 min at rt, thioglycerol (3.8 μL, 44 μmol, 3.0 equiv) and Et₃N (6.1 μL, 44 μmol, 3 equiv) were added. The reaction mixture was stirred at rt for another 17 h. The product was precipitated by slow addition to a stirring solution of EtOH/Et₂O (1:1, 2 mL). The precipitate was filtered off, washed with EtOH and dried. Further purification was achieved by ultrafiltration (Sartorius Stedim Vivaspin tubes, 6 mL, molecular weight cutoff 50 kDa, 5000 rpm). Freeze-drying gave the Tn antigen polymer 39 (4.0 mg, 65%) as a white solid. According to ¹H NMR, the product contained approximately 35% of the lysine side-chains substituted by the carbohydrate epitope 37.

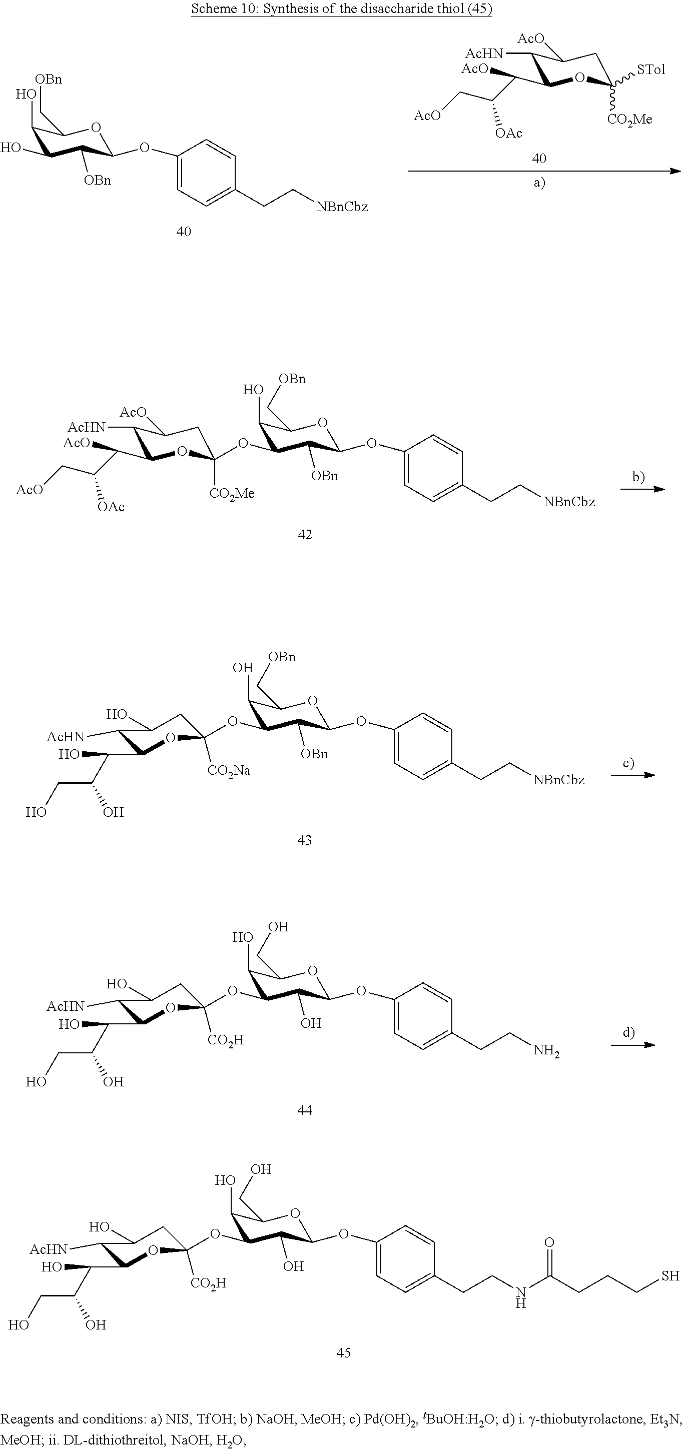

### Synthesis of the Disaccharide (42)

A solution of 41 Donor (1.06 g, 1.78 mmol) and 40 Acceptor (1.0 g, 1.42 mmol) in a 50% Acetonitrile CH₂Cl₂ mixture was stirred for 1 h with freshly dried molecular sieves (400 mg). N-Iodosuccinimide (415 mg, 1.85 mmol) was added, the suspension was stirred for 30 min and cooled to −40° C. TfOH (13 μL, 0.14 mmol) was added, the suspension was stirred over night at −40° C., diluted with CH₂Cl₂ (50 ml), filtered through a pad of Celite and the remaining solution was washed with diluted Na₂S₂O₃, sat. NaHCO₃, and brine. The organic layer was dried (Na₂SO₄), filtered, concentrated and the residue was purified on silica using a gradient from CH₂Cl₂:Acetone:2-Propanol 93:7:0.2 to CH₂Cl₂:Acetone:2-Propanol 80:20:0.2. Yield 42: 710 mg (42%); R_{f}: 0.51 (CH₂Cl₂:Acetone:2-Propanol 4:1:0.1).

¹H NMR (500 MHz, CDCl₃): δ ppm 7.43-6.87 (m, 24H, Ar—H), 5.41 (ddd, J=8.3, 6.0, 2.6 Hz, 1H, Neu5Ac—H8), 5.31 (dd, J=8.0, 2.0 Hz, 1H, Neu5Ac—H7), 5.25 (d, J=9.6 Hz, 1H, NH), 5.19 (s, 2H, CO₂CH₂Ar), 5.01 (br, 1H, Gal—H1), 4.90 (d, J=11.9 Hz, 2H, CH₂Ar), 4.86 (ddd, J=12.1, 10.0, 5.0 Hz, 1H, Neu5Ac—H4), 4.81 (d, J=11.8 Hz, 1H, CH₂Ar), 4.56 (d, J=1.7 Hz, 2H, CH₂Ar), 4.40 (br d, 2H, NCH₂Ar), 4.33 (dd, J=12.5, 1.9 Hz, 1H, Neu5Ac—H9a), 4.28 (dd, J=9.5, 2.8 Hz, 1H, Gal—H5), 4.04 (dd, J=10.7, 2.1 Hz, 1H, Neu5Ac—H5), 3.95 (dd, J=12.5, 6.0 Hz, 1H, Neu5Ac—H6), 3.85-3.74 (m, 5H, Gal—H2 Gal—H3 Gal—H4 Gal—H6), 3.79 (s, 3H, CO₂CH₃), 3.47-3.34 (m, 2H, Ar—CH₂), 2.76 (d, J=2.8 Hz, 1H, Gal—4OH), 2.74 (br m, 2H, CH₂N), 2.57 (dd, J=13.0, 4.6 Hz, 1H, Neu-H3e), 2.10 (s, 3H, COCH₃), 2.03 (t, J=12.4 Hz, 1H, Neu-H3a), 2.00 (s, 3H, COCH₃), 1.98 (s, 3H, COCH₃), 1.95 (s, 3H, COCH₃), 1.86 (s, 3H, COCH₃); MS (ESI-pos) m/z calcd for C₆₃H₇₂N₂O₂₀ (M+Na)⁺: 1199.46, found 1199.45

### Synthesis of the Disaccharide Amine (44)

Disaccharide 42 was dissolved in MeOH, NaOH (1M; 10 equiv) was added and the solution was stirred for 1 h. Additional H₂O was added until first turbidity appeared. The solution was stirred over night, neutralized until pH 7-8 with Acetic acid (20%). MeOH was removed under reduced pressure until first precipitation appeared. The suspension was purified on RP8 silica (10-90% Acetonitrile in H₂O). The product fraction was concentrated and the residue was dissolved in a 2:1 mixture of t-Butanol-H₂O. Acetic acid (20% in H₂O ; 35 equiv.) and Pd(OH)₂ on charcoal were added and the suspension was hydrogenated for 17 h at ambient pressure under vigorous stirring. The suspension was filtered, concentrated and co-evaporated with toluene. Yield 41% over two steps.

¹H NMR (500 MHz, D₂O): δ ppm 7.31 (d, J=8.7 Hz, 2H, Ar—H), 7.15 (d, J=8.7 Hz, 1H, Ar—H), 5.17 (d, J=7.9 Hz, 1H, Gal-H1), 4.23 (dd, J=9.8, 3.2 Hz, 1H, Gal-H3), 4.05 (d, J=3.1 Hz, 1H, Gal-H4), 3.92-3.84 (m, 5H, Gal-H2 Gal-H5 Neu-H5 Neu-H8 Neu-H9a), 3.77 (d, J=6.1 Hz, 2H, Gal-H6), 3.72 (ddd, J=11.7, 10.0, 4.7 Hz, 1H, Neu-H4), 3.67 (dd, J=10.4, 1.7 Hz, 1H, Neu-H6), 3.64 (dd, J=11.4, 5.8 Hz, 1H, Neu-H9b), 3.61 (dd, J=8.8, 1.9 Hz, 1H, Neu-H7), 3.27 (t, J=7.2 Hz, 2H, CH₂—NH₃⁺), 2.98 (t, J=7.2 Hz, 2H, Ar—CH₂), 2.81 (dd, J=12.4, 4.6 Hz, 1H, Neu-H3e), 2.05 (s, 3H, COCH₃), 1.84 (t, J=12.1 Hz, 1H, Neu-H3a).The thiol 45 can be synthesized using similar procedure as mentioned before.

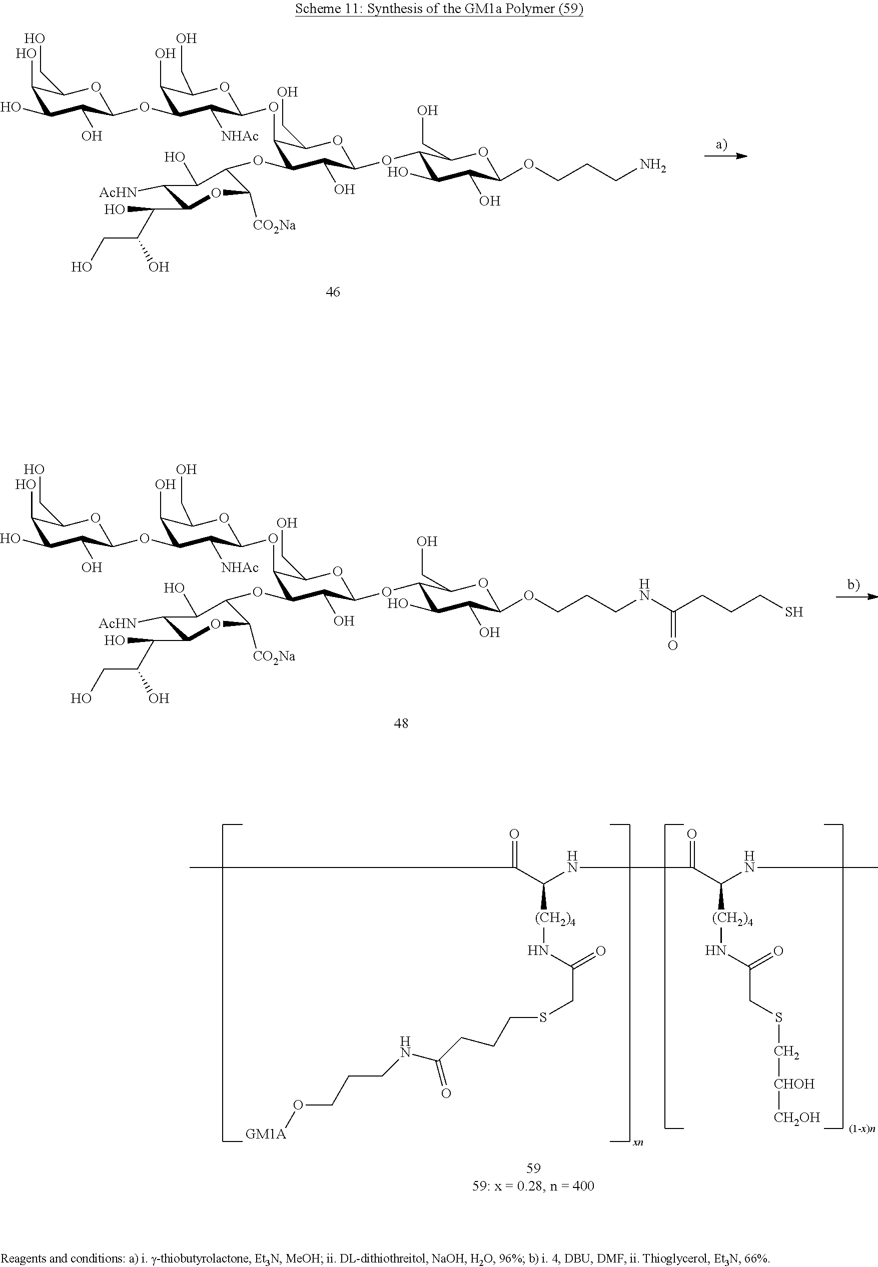

### Synthesis of the GM1a Thiol (48)

Amine 46 (20 mg, 18.6 μmol) was dissolved in water (0.300 ml) and 1M NaOH (19 □I, 18.6 μmol, equiv) was added. Thiobutyrolacton (10 equiv.) was added and MeOH (0.30 ml) was added until monophasic solution was obtained. The reaction was stirred for 3 h. Additional 1M NaOH (3.8 μL, 3.72 μmol, 0.2 equiv) was added and the reaction was stirred over night. TLC shows >95% product. One drop HAc (20%) was added, MeOH was removed under reduced pressure purified by RP column to obtain thiol (18.0 mg, 82%).

¹H NMR (500 MHz, D₂O): δ ppm 4.82-4.78 (m, 1H), 4.56 (d, J=7.3 Hz, 1H), 4.55 (d, J=7.1 Hz, 1H), 4.50 (d, J=7.9 Hz, 1H), 4.17 (d, J=14.3 Hz, 3H), 4.06 (dd, J=10.5, 8.8 Hz, 1H), 4.03-3.57 (m, 28H), 3.53 (dd, J=17.1, 8.0 Hz, 2H), 3.35 (ddd, J=24.8, 15.3, 9.0 Hz, 4H), 2.68 (dd, J=12.5, 4.0 Hz, 1H), 2.56 (t, J=7.1 Hz, 2H), 2.38 (t, J=7.4 Hz, 2H), 2.05 (s, 3H), 2.02 (s, 3H), 1.98-1.81 (m, 5H).

### Synthesis of the GM1a Polymer (59)

A solution of polymer 4 (5.0 mg, 24.4 μmol) in DMF (244 μL) was added to thiol 48 (11.5 mg, 9.8 μmol, 0.4 equiv). A solution of DBU (3.6 μL, 24.4 μmol, 1.0 equiv) was successively added to the reaction mixture. After stirring for 60 min at rt, thioglycerol (6.3 μL, 73.3 μmol, 3.0 equiv) and Et₃N (10.2 μL, 73.3 μmol, 3 equiv) were added. The reaction mixture was stirred at rt for another 17 h. The product was precipitated by slow addition to a stirring solution of EtOH/Et₂O (1:1, 5 mL). The precipitate was filtered off, washed with EtOH and dried. Further purification was achieved by ultrafiltration (Sartorius Stedim Vivaspin tubes, 6 mL, molecular weight cutoff 50 kDa, 5000 rpm). Freeze-drying gave the GM1a polymer 59 (9.0 mg, 62%) as a white solid. According to ¹H NMR, the product contained approximately 28% of the lysine side-chains substituted by the carbohydrate epitope 48.

Competitive Binding Assay with Blood Group A/B Agluttinins and Shiga Toxin B Subunit

The synthesized carbohydrate polymers 9 and 32 (A antigen), 35 (B antigen), 5 and 6 (Gb3 epitope), and 23 (Gb3 epitope mimetic) were tested with competitive ELISA, using maleimide activated plates (Thermo Scientific). The 96 well microtiter plates were washed three times with wash buffer (0.1 M sodium phosphate, 0.15 M sodium chloride, 0.05% Tween-20, pH 7.2), 200 μl/well, and then incubated with 100 μl/well of a 1-50 μg/ml solution of the respective sulfhydryl-containing epitopes in binding buffer (0.1M sodium phosphate, 0.15M sodium chloride, 10 mM EDTA, pH 7.2) overnight at 4° C. After washing three times with wash buffer (200 μl/well) the plates were inclubated with 200 μl/well of a freshly prepared 10 μg/ml cysteine solution for 1 h at room temperature. After washing three times with wash buffer (200 μl/well) the plates were incubated with the systhesized carbohydrate polymers at different concentrations, 25 μl/well (2× concentrated), and the relevant carbohydrate binding protein (CBP) at a suitable concentration/dilution, 25 μl/well (2× concentrated), for 1 h at room temperature. The wells were washed three times with wash buffer (200 μl/well) before the secondary antibody-horseradish peroxidase conjugate was added (100 μl/well). The plate is incubated for 1 h at room temperature. After washing the wells (200 μl/well), a substrate solution of tetramethylbenzidin (Thermo Scientific, 34028) was added (100 μl/well) and the plate incubated for further 5-30 minutes at room temperature, protected from light. Finally, a stop solution (1M sulfuric acid) was added (100 μl/well) and the degree of colorimetric reaction was determined by absorption measurement at 450 nm with a microplate reader (Spectramax 190, Molecular Devices, California, USA). The IC₅₀ values of the tested compounds were calculated using Prism® software (GraphPad Prism 5.0, Inc, La Jolla, USA).

Competitive Binding Assay with an Anti-Blood Group A (BGA) Agluttinin

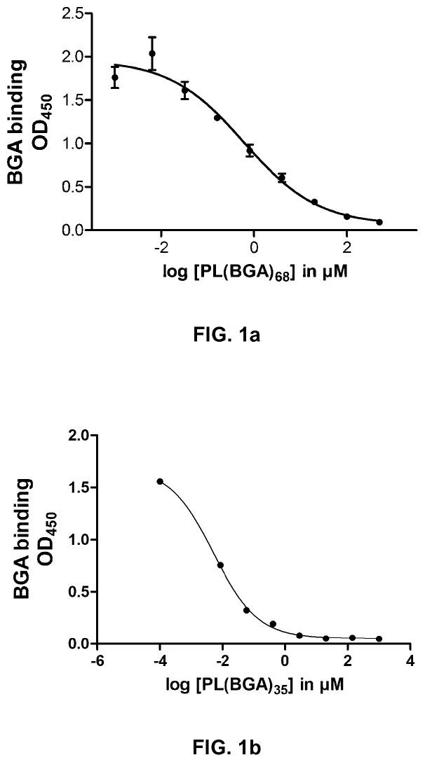

The carbohydrate polymers 9 and 32 (both BGA epitopes) were tested in a competitive binding assay at concentrations of 6.4 nM to 0.5 mM and 0.1 nM to 1 mM respectively, co- incubated with anti-BGA agluttinin (Sigma Aldrich, SAB4700674, clone HE-193) at a dilution of 1:100 in binding buffer. Goat anti-mouse IgM HRP conjugate (Sigma Aldrich, A8786), diluted 1:10,000, was used as secondary antibody. The measured IC₅₀ was 0.6 μM and 5.2 nM respectively (FIG. 1a and 1b).

Competitive Binding Assay with an Anti-Blood Group B (BGB) Agluttinin

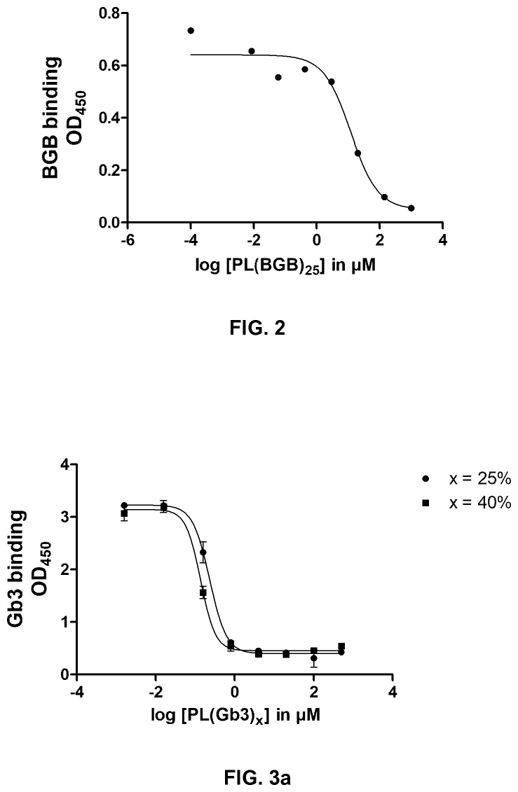

The carbohydrate polymer 35 (BGB epitope) was tested in a competitive binding assay at concentrations of 0.1 nM to 1 mM, co-incubated with anti-BGB agluttinin (Sigma Aldrich, SAB4700676, clone HEB-29) at a dilution of 1:10 in binding buffer. Goat anti-mouse IgM HRP conjugate (Sigma Aldrich, A8786), diluted 1:10,000, was used as secondary antibody. The measured IC₅₀ was 11.9 μM (FIG. 2).

Competitive Binding Assay with the Shiga Like Toxin 1 B Subunit

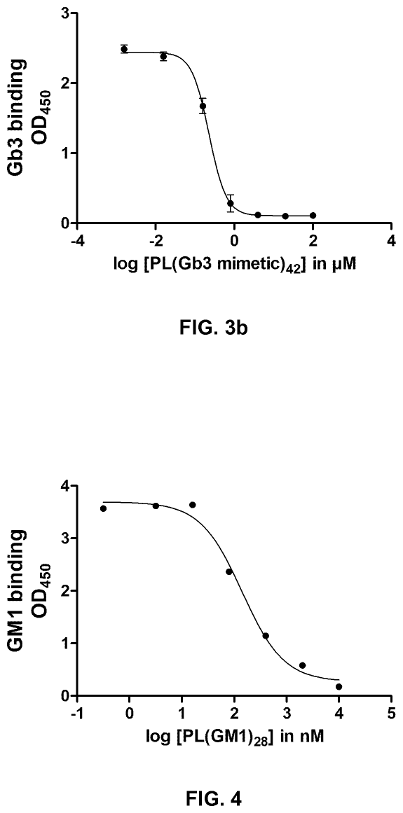

The carbohydrate polymers 5, 6 (Gb3 epitope), and 23 (Gb3 epitope mimetic) were tested in a competitive binding assay at concentrations of 1.6 nM to 500 μM, co-incubated with the HIS-tagged Shiga like toxin 1 B subunit (Biozol, MBS145496) at a concentration of 2 μg/ml. Mouse anti-HIS HRP conjugate (ThermoFischer Scientific, MA1-21315-HRP), at a dilution of 1:10,000, was used as secondary antibody. The obtained IC₅₀ values were 233.3 nM for polymer 5, 138.0 nM for polymer 6, and 229.0 nM for polymer 23 (FIGS. 3a and 3b).

Competitive Binding Assay with Cholera Toxin B Subunit

The synthesized carbohydrate polymer 59 (GM1 antigen), was tested with competitive ELISA, using an anti-GM1 ELISA (Bühlmann Laboratories, Schönenbuch, Switzerland). The 96 well microtiter plates, coated with the GM1 ganglioside, were washed two times with washing buffer (300 μl/well) before co-incubation of the carbohydrate polymer at concentrations from 0.32 nM to 10 μM and cholera toxin-B subunit-HRP conjugate (Sigma Aldrich, C3741) at a concentration of 0.5 μg/ml (total volume of 50 μl/well). After an incubation for 2 h at RT the wells were washed three times with wash buffer (300 μl/well) before a substrate solution of tetramethylbenzidin (Thermo Scientific, 34028) was added (100 μl/well) and the plate incubated for further 5 minutes at 600 rpm at RT, protected from light. Finally, a stop solution (1M sulfuric acid) was added (100 μl/well) and the degree of colorimetric reaction was determined by absorption measurement at 450 nm with a microplate reader (Spectramax 190, Molecular Devices, California, USA). The IC₅₀ value was determined using Prism® software (GraphPad Prism 5.0, Inc, La Jolla, USA) and was 103.7 nM for polymer 59 (FIG. 4).

Binding Assay with an Anti-Tn Immunoglobulin

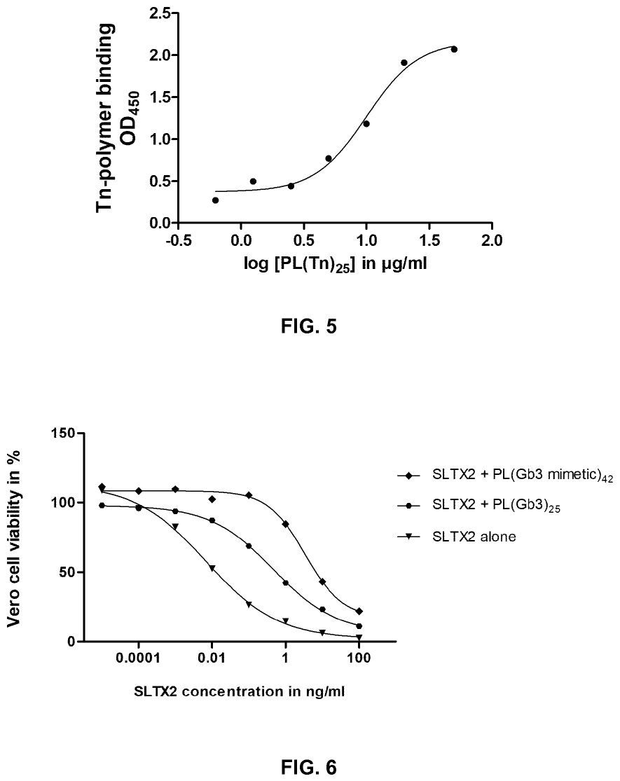

The carbohydrate polymer 38 (Tn epitope) was tested in a binding ELISA. Maxisorp plates (Thermo Scientific, 442404) were coated overnight with polymer 38 at concentrations of 0.6 μg/ml to 50.0 μg/ml (50 μl/well) at 4° C. The plates washed three times with wash buffer (PBS, 0.1% Tween), 200 μl/well. The coated plates were blocked for 2 h at RT with 100 μl/well of 5% BSA in PBS, 0.1% Tween. The blocking solution was discarded and 50 μl/well of a mouse anti-Tn IgM antibody (reBaGs6, Beth Israel Deaconess Medical Center Glycomics Core) at a dilution of 1:700. After incubating for 2 h at RT the wells were washed three times with wash buffer (200 μl/well). Then, as secondary antibody, 100 μl/well of HRP-labeled anti-mouse IgM (Sigma Aldrich, A8786) was incubated for 2 h at RT. The wells were washed three times with wash buffer (200 μl/well) before a substrate solution of tetramethylbenzidin (Thermo Scientific, 34028) was added (100 μl/well) and the plate incubated for further 30 minutes at 600 rpm at RT, protected from light. Finally, a stop solution (1M sulfuric acid) was added (100 μl/well) and the degree of colorimetric reaction was determined by absorption measurement at 450 nm with a microplate reader (Spectramax 190, Molecular Devices, California, USA). The EC₅₀ value was determined using Prism® software (GraphPad Prism 5.0, Inc, La Jolla, USA) and was 10.0 μg/ml for polymer 38 (FIG. 5).

Cell Viability Assay with Shiga Like Toxin 2

The protective effect of polymers 5 and 23 on cytotoxic damage of vero cells (Creative Bioarray, CSC-C8963H) by Shiga like toxin 2 (List Biological Laboratories Inc., Prod. Nr. 164, Lot: 1645A1) was tested in a cell viability assay. Vero cells, a kidney cell line from cercopithecus aethiops (monkey, african green) expressing the Gb3 receptor, were maintained in culture medium (MEM Eagle (Sigma, M4655, RNBF9153), 10% FBS (Gibco, 10500064), 1% (v/v) non-essentiaon amino acid solution (Sigma, M7145, RNBF6784), 1% (v/v) sodium pyruvate (Sigma, S8636), 1% (v/v) antibiotic-antimycotic solution (Gibco, 15240062)). For the viability assay, the vero cells were grown overnight in serum-free culture medium in 96 well plates (5000 cells/well) at 37° C., 5% CO₂. Then, the medium was discarded, and the cells were incubated for 48 h at 37° C., 5% CO₂ with 100 μl/well of serum-free culture medium containing Shiga like toxin 2 at concentrations of 0.00001 to 100 μg/ml and the polymers 5 or 23 at a concentration of 30 μg/ml. Thereafter, 20 μl/well of CellTiter Blue® assay reagent (Promega, G8080, 258569) was added and the plate incubated for 4 h at 37° C., 5% CO₂. The fluorescent signal (viable cells transform a non-fluorescent educt in a fluorescent product) was then read using a Synergy HT fluorometer (Ex: 520/25, Em: 590/20). The signal curves were fittet and EC₅₀ values determined using Prism® software (GraphPad Prism 5.0, Inc, La Jolla, USA). The EC₅₀ (the concentration at which 50% of the vero cells remain viable) of Shiga like toxin 2 was determined at 6.9 ng/ml. The EC₅₀ of Shiga like toxin 2 co-incubated with polymer 5, with 25% loading of a natural Gb3 epitope, was 464.9 ng/ml and the EC₅₀ of Shiga like toxin 2 co-incubated with polymer 23, with 42% loading of a Gb3 epitope mimetic, was 3434.0 ng/ml (FIG. 6).

The inventive polymers 5, 6, 23 and 59 are carbohydrate polymers which imitate the glycoepitopes of the Gb3- and the GM1-glycolipid. The polymers 5 and 6 display the natural Gb3 epitope, whereas polymer 23 displays a Gb3 mimetic. Gb3 is the natural receptor for bacterial Shiga toxins which are a major pathogenic factor in infections with shiga-toxin producing bacteria (Shigella or E. coli) or in hemolytic-uremic syndrome (HUS). The polymer 59 displays the natural GM1 glecoepitope which is the receptor for the bacterial cholera toxin which is a major pathogenic factor in infections with Vibrio cholerae. ELISA tests showed that said polymers can be used to inhibit the binding of the Shiga like toxin 1 B subunit to the natural Gb3 receptor (polymers 5, 6, and 23; FIG. 3a,b) and to inhibit the binding of the Cholera toxin B subunit to the natural GM1 receptor (polymer 59; FIG. 4), with IC₅₀ values in the nanomolar range. The B subunits are the ones which promote the critical cell-adhesion, which is a prerequisite to then induce cytotoxic damage via the A subunit of the AB₅-type toxins. A cell viability assay with vero cells further substanciated the usefulness of polymers 5 and 23 to prevent cytotoxic damage from Shiga like toxin 2 (containing both the B and A subunits) towards vero kidney cells, which highly express the natural Gb3 receptor (FIG. 6). Treatment of vero cells with 30 μg/ml of these two glycopolymers had a protective effect, leading to an increase in the required toxin concentration by a factor of roughly 70 in case of polymer 5 and by a factor of roughly 500 in case of polymer 23 to have the same cytotoxic damage to vero cells compared to treatment of vero cells with the toxin alone. The results suggest that such polymers may be used in a therapeutic setting to bind to the B subunits of said bacterial toxins and thus prevent their cytotoxic damage.

The inventive polymers 9, 32 and 35 are cyrbohydrate poylmers which imitate the A and the B antigens which are found e.g. on red blood cells. The polymers 9 and 32 both display an A-type carbohydrate antigen, polymer 35 displays a B-type carbohydrate antigen. ELISA tests showed that polymers 9 and 32 can be used to inhibit the binding of anti-A agluttinin to the A carbohydrate antigen (FIG. 1a,b) and that polymer 35 can be used to inhibit the binding of anti-B agluttinin to the B carbohydrate antigen (FIG. 2). The results suggest that such polymers may be used in a therapeutic setting to prevent the damage that agluttinins can cause in ABO-incompatible transplantations.

The inventive polymer 38 is a carbohydrate polymer which imitates the Tn antigen. Immunoglobulins against the Tn antigen are involved in immune complex formation in diseases such as IgA nephropathy and IgA vasculitis. An ELISA test showed that polymer 38 can bind an antibody, that was raised against the Tn antigen, in a concentration-dependent manner (FIG. 5). The results suggest that such a polymer could be used in a therapeutic setting to bind/inhibit immunoglobulins against the Tn antigen and thus prevent immune complex formation.

The prepared carbohydrate polymers are based on a biodegradable poly-L-lysine backbone and are designed for a therapeutic application in patients, where pathogenic carbohydrate-binding proteins, binding the above-mentioned carbohydrate epitopes, could be selectively neutralized and removed by these polymers which display the same or similar carbohydrate epitopes.

1. A polymer comprising a multitude of a compound, wherein said compound comprises a carbohydrate moiety and a linker Z, and wherein
said carbohydrate moiety mimics a glycoepitope recognized by a carbohydrate-binding protein (CBP), wherein said CBP is selected from a bacterial exotoxin, an agluttinin and an immune complex deposit-forming immunoglobulin, and wherein
said linker Z is —X-A-(B)ₚ-(CH₂)_{q}—Y, wherein
X is O or N(R^{a});
R^{a} is H, C₁₋₄alkyl, C₁₋₄alkoxy, CH₂C₆H₅, CH₂CH₂C₆H₅, OCH₂C₆H₅, or OCH₂CH₂C₆H₅;
A is C₁₋₇alkylene, OC₁₋₇alkylene, C₁₋₄alkylene-(OCH₂CH₂)ᵣOC₁₋₄alkylene, OC₁₋₇alkylene-R^{b}, or R^{b}-C₁₋₇alkylene wherein R^{b} is an optionally substituted aryl or an optionally substituted heteroaryl, and wherein r is 0 to 6, preferably r is 1, 2 or 3, and further preferably r is 1;
B is NHC(O) or S;
p is 0 or 1;
q is 0 to 6, preferably q is 0 to 4, and further preferably q is 0, 2 or 3;
Y is SH, N₃ or NH₂; and
wherein said linker Z is covalently bound via its —X-group to the reducing end of said carbohydrate moiety; and wherein
said multitude of said compound is connected to the polymer backbone by way of said linker Z, and wherein said connection is effected via the Y-group of said linker Z; and
wherein said compound is not 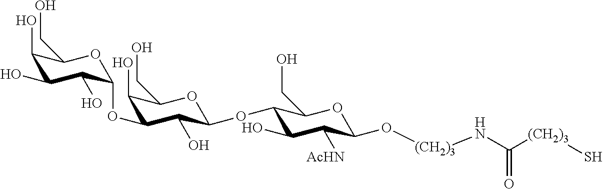
when said polymer backbone is poly-L-lysine.

2. The polymer of claim 1, wherein said compound is a compound of formula (I), formula (II), formula (III) or formula (IV),
wherein formula (I) is 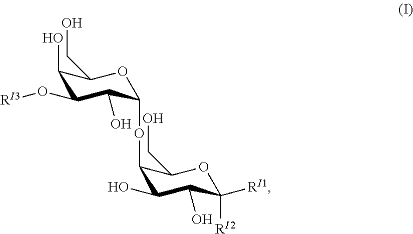
wherein R^{I1} is H or Z or 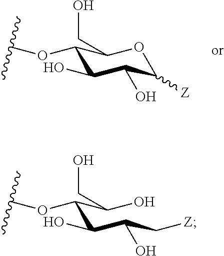
wherein R^{I2} is H or Z;
wherein, when R^{I1} is H, then R^{I2} is Z; and when R^{I1} is not H, then R^{I2} is H;
and wherein R^{I3} is H or 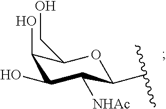
wherein formula (II) is 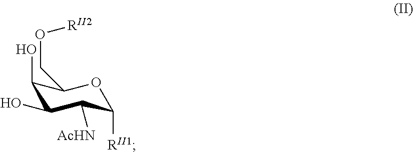
wherein R^{III1} is Z or 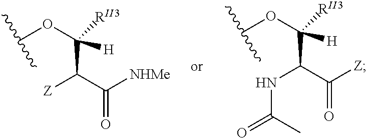
wherein R^{II2} is H or 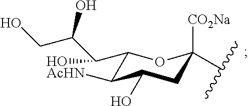
and wherein R^{II3} is H or Me;
wherein formula (III) is 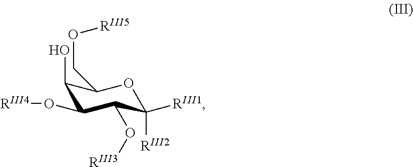
wherein R^{III1} is H or Z or 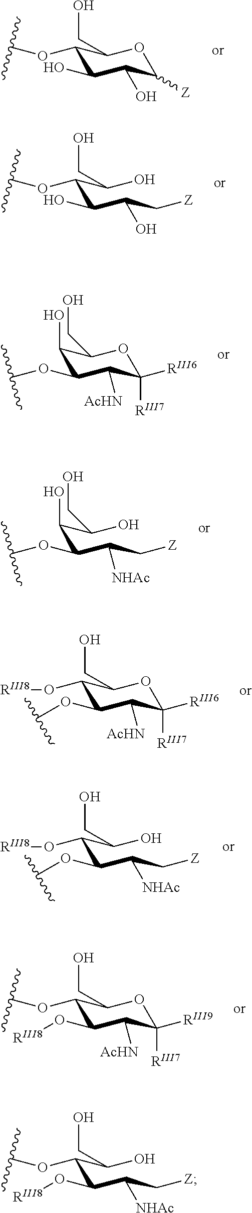
wherein R^{III2} is H or Z;
wherein when R^{III1} is H, then R^{III2} is Z; and when R^{III1} is not H, then R^{III2} is H;
wherein R^{III3} and R^{III8} are independently H or 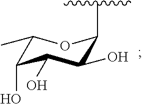
wherein when R^{III1} is not 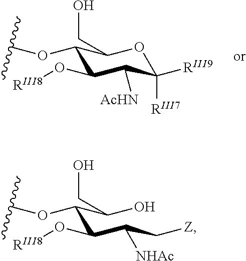
then R^{III3} is H;
wherein R^{III4} is H or 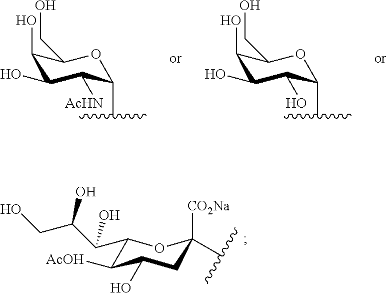
wherein when R^{III4} is not H, then R^{III8} is H;
wherein R^{III3} is H, then R^{III4} is H
wherein R^{III5} is H or 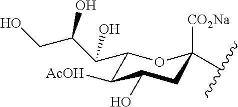
wherein when R^{III4} or R^{III5} is 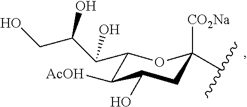
then R^{III1} is H, Z or 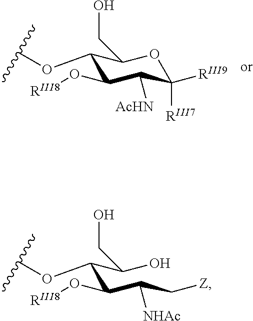
and R^{III3} and R^{III8} are H;
wherein R^{III6} is H or Z or 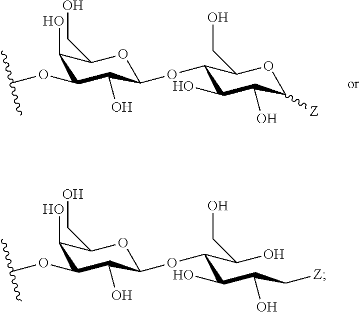
wherein R^{III7} is H or Z;
wherein when R^{III6} is H, then R^{III7} is Z and when R^{III6} is not H, then R^{III7} is H;
wherein R^{III9} is H or Z or 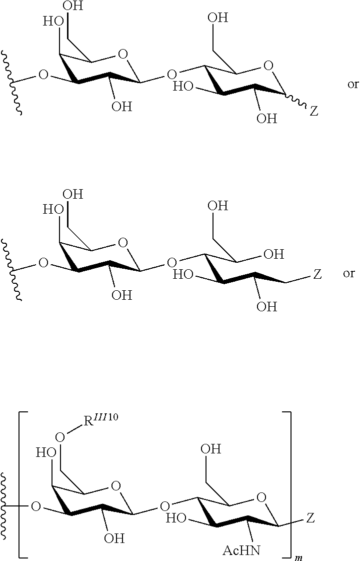
wherein m is 1 to 3;
wherein when R^{III9} is 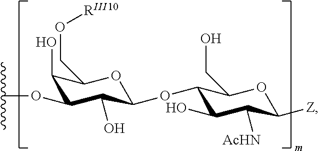
then R^{III3} R^{III4}, R^{III5} and R^{III8} are H;
wherein R^{III10} is H orl 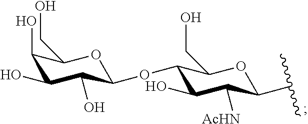
wherein formula (IV) is 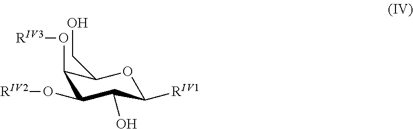
wherein R^{IV1} is 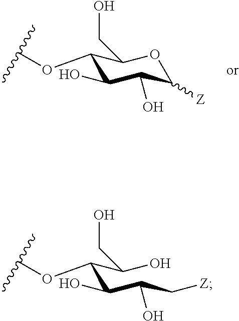
wherein R^{IV2} and R^{IV4} are independently H or 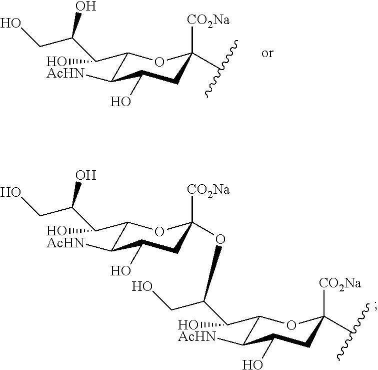
wherein R^{IV3} is H or 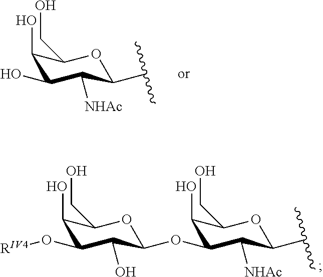
wherein when said compound is a compound of formula (IV), then said linker Z is not —N(R^{a})-A-B-CH₂—(CH₂)_{q}—SH, wherein
R^{a} is H, C₁-C₄-alkyl, C₁-C₄-alkoxy, CH₂C₆H₅, CH₂CH₂C₆H₅, OCH₂C₆H₅, or OCH₂CH₂C₆H₅;
A is C₁₋₇alkylene, C₁-C₇-alkoxy, C₁₋₄alkylene-(OCH₂CH₂)ᵣO—C₁₋₄alkylene or C₁-C₇-alkoxy-R^{b}, wherein R^{b} is an optionally substituted aryl or an optionally substituted heteroaryl, and wherein r is 0 to 6, preferably r is 1, 2 or 3, and further preferably r is 1;
B is NHC(O), S or CH2;
q is 0 to 6, preferably q is 1, 2, 3 or 4, and further preferably q is 1 or 2;
In a further preferred embodiment, said compound is a compound of formula (I). In a further preferred embodiment, said compound is a compound of formula (II). In a further preferred embodiment, said compound is a compound of formula (III). In a further preferred embodiment, said compound is a compound of formula (IV).

3. The polymer of any one of claim 1 or 2, wherein said compound is a compound of any one of formula 3*, 8*, 22*, 26*, 31*, 34*, 37*, 45*, 47*-58*.

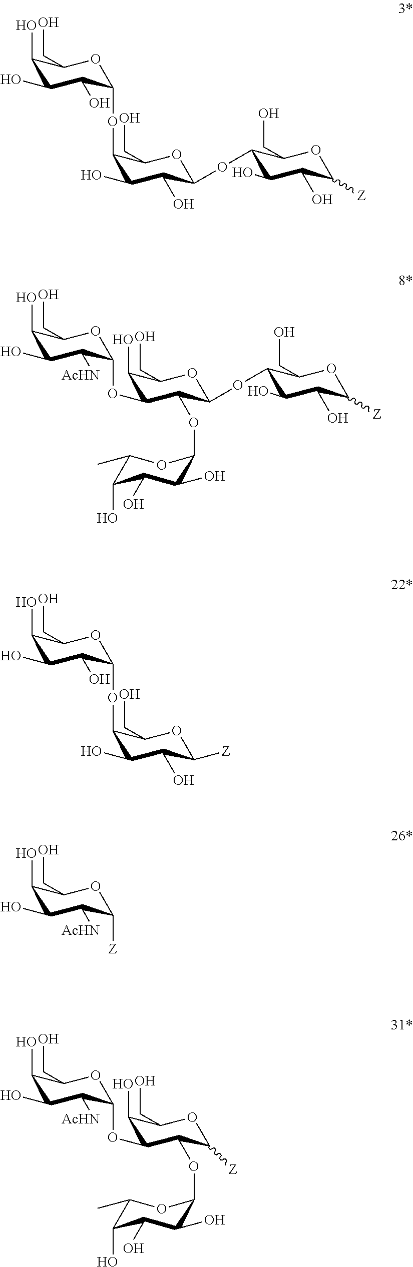

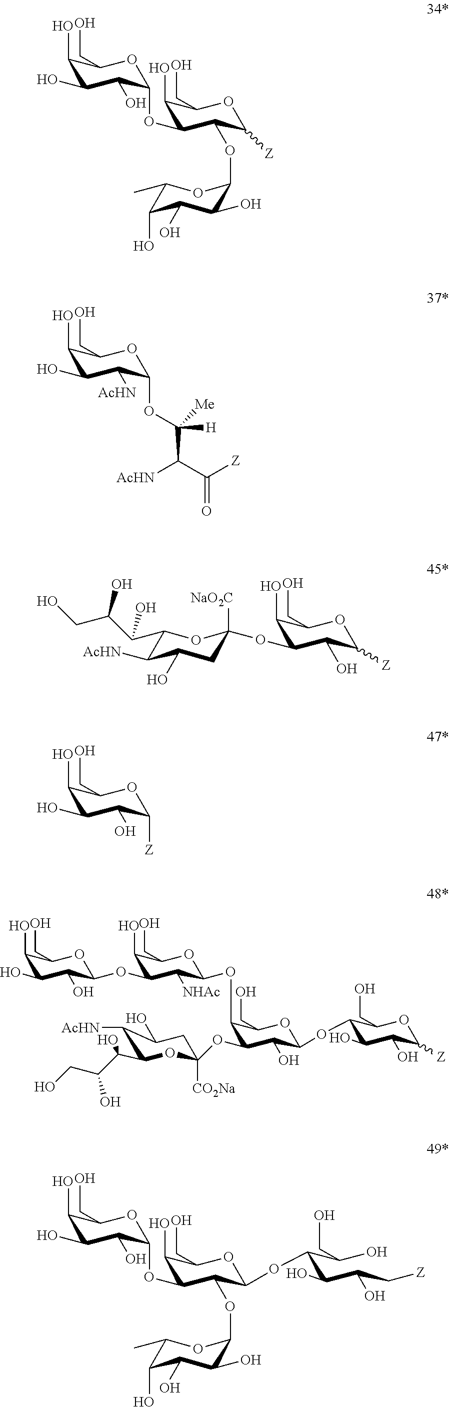

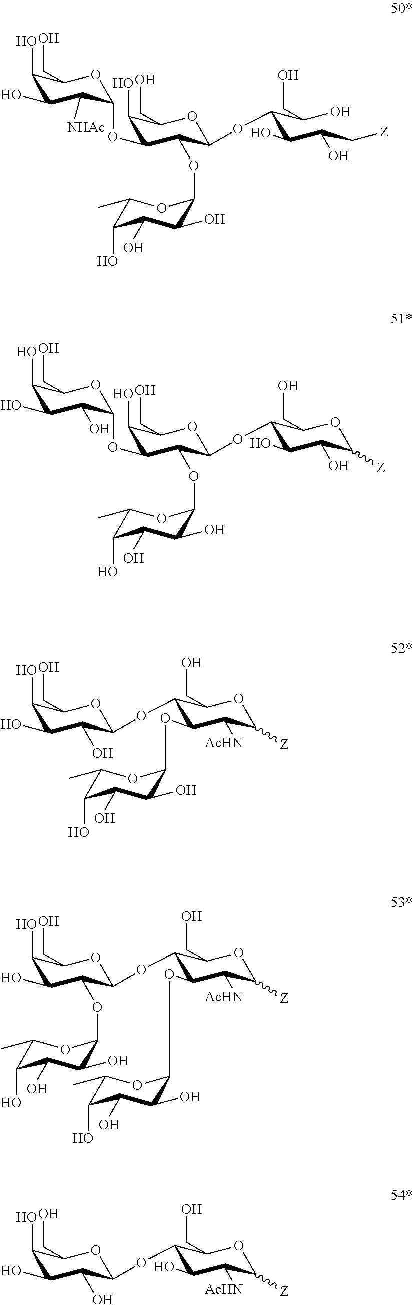

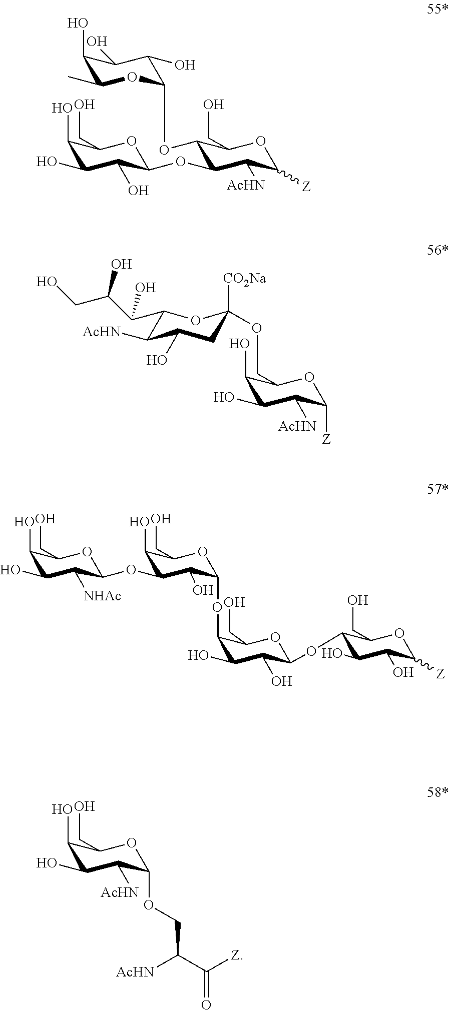

4. The polymer of any one of claims 1 to 3, wherein said linker Z is —X-A-(B)ₚ-(CH₂)_{q}—Y, wherein
X is N(R^{a});
R^{a} is H, C₁₋₄alkyl, C₁₋₄alkoxy, CH₂C₆H₅, CH₂CH₂C₆H₅, OCH₂C₆H₅, or OCH₂CH₂C₆H₅;
A is C₁₋₇alkylene, OC₁₋₇alkylene, C₁₋₄alkylene-(OCH₂CH₂)ᵣO—C₁₋₄-alkylene, OC₁₋₇alkylene-R^{b} or R^{b}—C₁₋₇alkylene wherein R^{b} is an optionally substituted aryl or an optionally substituted heteroaryl, and wherein r is 0 to 6, preferably r is 1, 2 or 3, and further preferably r is 1;
B is NHC(O) or S;
p is 0 or 1;
q is 0 to 6, preferably q is 0 to 4, and further preferably q is 0, 2 or 3;
Y is SH, N₃ or NH₂.

5. The polymer of any one of claims 1 to 4, wherein said linker Z is —X-A-(B)ₚ-(CH₂)_{q}—Y, wherein
X is O;
A is C₁₋₇alkylene, C₁₋₄alkylene-(OCH₂CH₂)ᵣOC₁₋₄alkylene or R^{b}—C₁₋₇alkylene, wherein R^{b} is an optionally substituted aryl or an optionally substituted heteroaryl, and wherein r is 0 to 6, preferably r is 1, 2 or 3, and further preferably r is 1;
B is NHC(O) or S;
p is 0 or 1, and wherein preferably p is 1;
q is 0 to 6, preferably q is 0 to 4, and further preferably q is 0, 2 or 3;
Y is SH, N₃ or NH₂.

6. The polymer of any one of the preceding claims, wherein said linker Z is of a formula selected from any one of the formula (a) to (g): 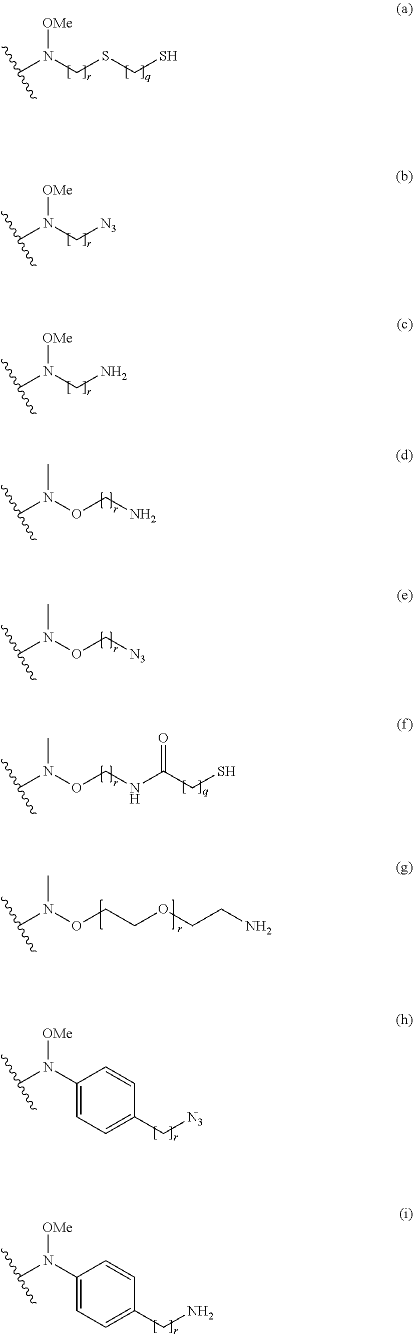 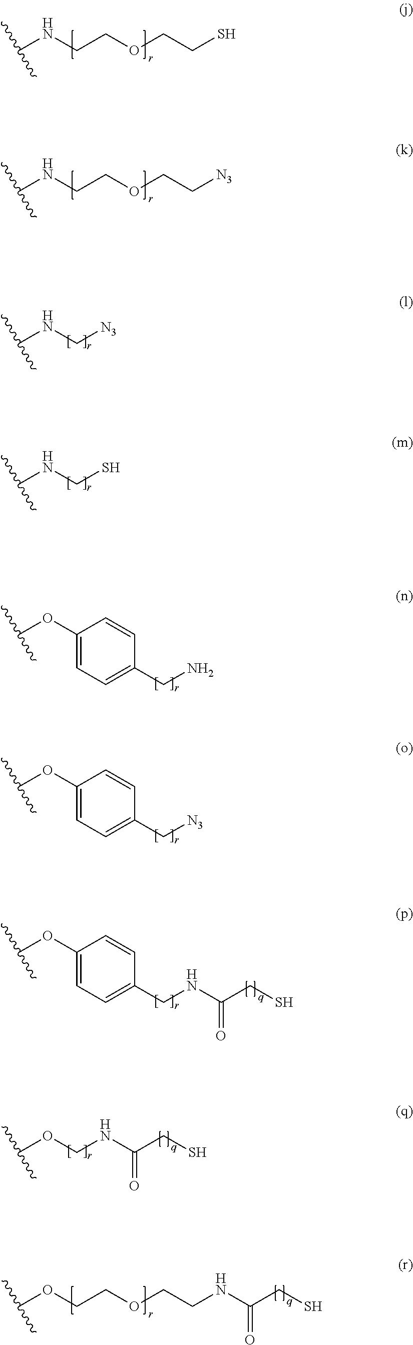
wherein r is 0 to 6, preferably 1 to 3, in particular 1, and q is 0 to 6, preferably 1, 2 and 4, in particular 1 or 2.

7. The polymer of claim 1, wherein said compound is a compound of formula 3, 8, 22, 26, 31, 34, 37, 45 or 48.

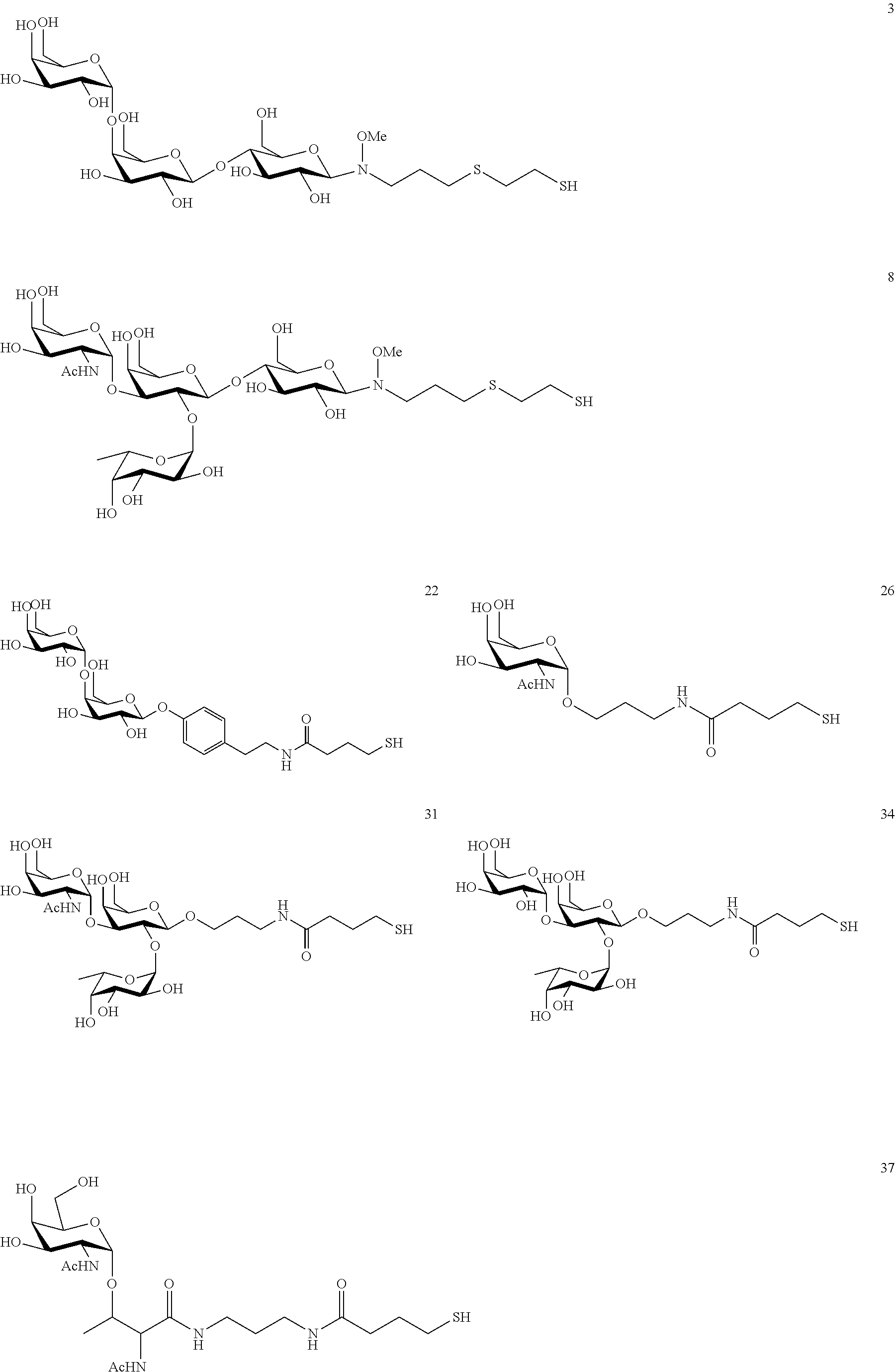

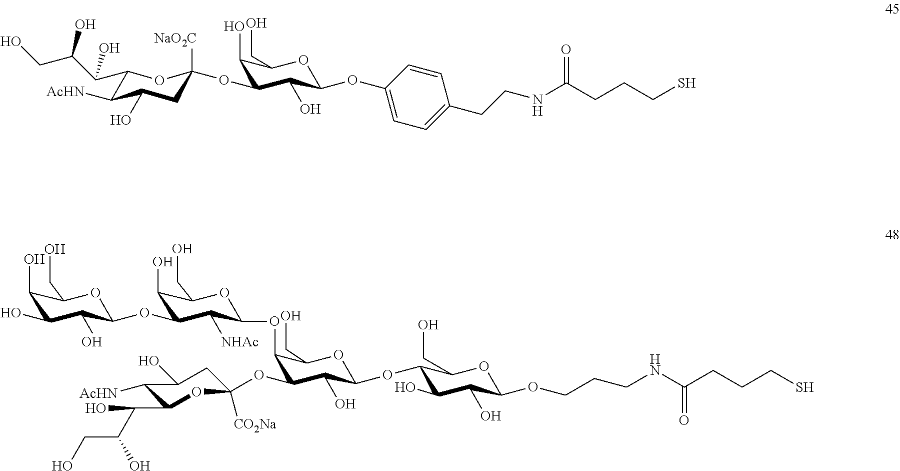

8. The polymer of any one of the preceding claims, wherein said polymer backbone is an α-amino acid polymer, an acrylic acid or methacrylic acid polymer or copolymer, a N-vinyl-2-pyrrolidone-vinyl alcohol copolymer, a chitosan polymer, or a polyphosphazene polymer, wherein preferably said polymer backbone is an α-amino acid polymer, and wherein further preferably said α-amino acid of said α-amino acid polymer is lysine, ornithine, glutamine, asparagine, glutamic acid or aspartic acid.

9. The polymer of any one of the preceding claims, wherein the polymer backbone is poly-lysine, and wherein preferably the molecular weight of the polymer backbone is 1,000 Da to 300,000 Da, further preferably the molecular weight of the polymer backbone is 30,000 to 150,000 Da.

10. A compound comprising a carbohydrate moiety and a linker Z, wherein said carbohydrate moiety mimics a glycoepitope recognized by a carbohydrate-binding protein (CBP), wherein said carbohydrate moiety mimics a glycoepitope recognized by a carbohydrate-binding protein (CBP), wherein said CBP is selected from a bacterial exotoxin, an agluttinin and an immune complex deposit-forming immunoglobulin, and wherein said linker Z is —X-A-(B)ₚ-(CH₂)_{q}—Y, wherein
X is O or N(R^{a});
R^{a} is H, C₁₋₄alkyl, C₁₋₄alkoxy, CH₂C₆H₅, CH₂CH₂C₆H₅, OCH₂C₆H₅, or OCH₂CH₂C₆H₅;
A is C₁₋₇alkylene, OC₁₋₇alkylene, C₁₋₄alkylene-(OCH₂CH₂)ᵣOC₁₋₄alkylene, OC₁₋₇alkylene-R^{b}, or R^{b}—C₁₋₇alkylene wherein R^{b} is an optionally substituted aryl or an optionally substituted heteroaryl, and wherein r is 0 to 6, preferably r is 1, 2 or 3, and further preferably r is 1;
B is NHC(O) or S;
p is 0 or 1;
q is 0 to 6, preferably q is 0 to 4, and further preferably q is 0, 2 or 3;
Y is SH, N₃ or NH₂; and
wherein said linker Z is covalently bound via its —X-group to the reducing end of said carbohydrate moiety.

11. The compound of claim 10, wherein said linker Z is —X-A-(B)ₚ-(CH₂)_{q}—Y, and wherein when X is N(R^{a}); then
R^{a} is H, C₁₋₄-alkyl, C₁₋₄-alkoxy, CH₂C₆H₅, CH₂CH₂C₆H₅, OCH₂C₆H₅, or OCH₂CH₂C₆H₅;
A is C₁₋₇alkylene, OC₁₋₇alkylene, C₁₋₄alkylene-(OCH₂CH₂)ᵣOC₁₋₄alkylene, OC₁₋₇alkylene-R^{b}, or R^{b}—C₁₋₇alkylene wherein R^{b} is an optionally substituted aryl or an optionally substituted heteroaryl, and wherein r is 0 to 6, preferably r is 1, 2 or 3, and further preferably r is 1;
B is NHC(O) or S;
p is 0 or 1;
q is 0 to 6, preferably q is 0 to 4, and further preferably q is 0, 2 or 3;
Y is SH, N₃ or NH₂; and wherein
when X is O; then
A is R^{b}—C₁-C₇-alkylene wherein R^{b} is an optionally substituted aryl or an optionally substituted heteroaryl, and wherein r is 0 to 6, preferably r is 1, 2 or 3, and further preferably r is 1;
B is NHC(O) or S;
p is 0 or 1, preferably p is 1;
q is 0 to 6, preferably q is 0 to 4, and further preferably q is 0, 2 or 3;
Y is SH, N₃ or NH₂.

12. The compound of claim 10 or claim 11, wherein said compound is a compound of formula (I), formula (II), formula (III) or formula (IV),
wherein formula (I) is 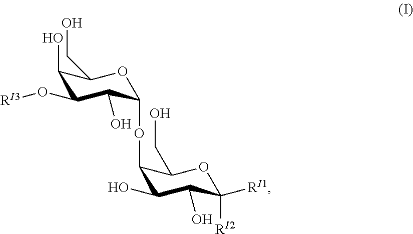
wherein R^{I1} is H or Z or 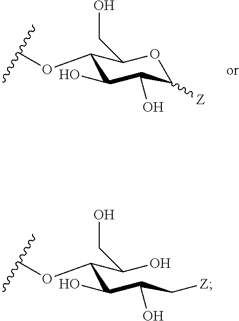
wherein R^{I2} is H or Z;
wherein, when R^{I1} is H, then R^{I2} is Z; and when R^{I1} is not H, then R^{I2} is H;
and wherein R^{I3} is H or 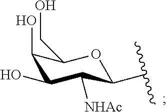
wherein formula (II) is 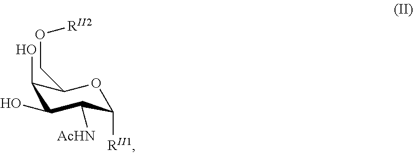
wherein R^{II1} is Z or 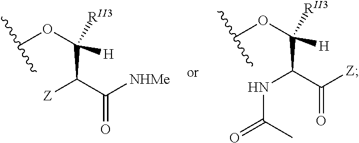
wherein R^{II2} is H or 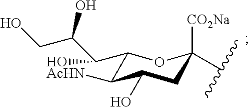
and wherein R^{II3} is H or Me;
wherein formula (III) is 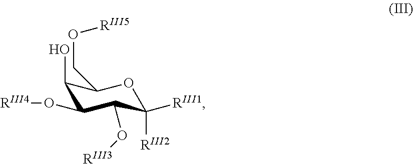
wherein R^{III1} is H or Z or 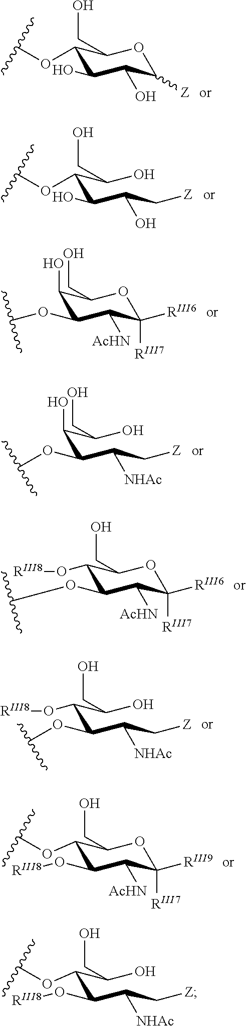
wherein R^{III2} is H or Z;
wherein when R^{III1} is H, then R^{III2} is Z; and when R^{III1} is not H, then R^{III2} is H;
wherein R^{III3} and R^{III8} are independently H or 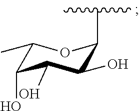
wherein when R^{III1} is not 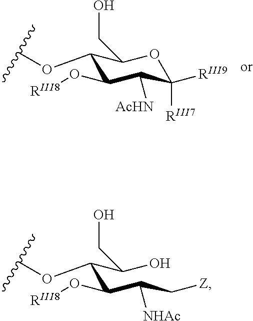
then R^{III3} is H;
wherein R^{III4} is H or 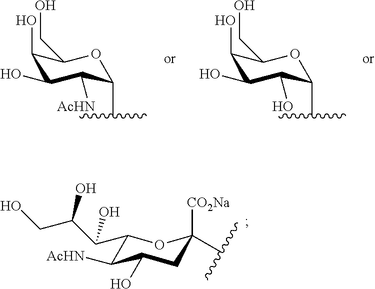
wherein when R^{III4} is not H, then R^{III8} is H;
wherein R^{III3} is H, then R^{III4} is H
wherein R^{III5} is H or 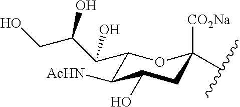
wherein when R^{III4} or R^{II5} is 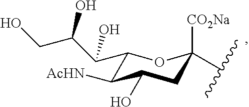
then R^{III1} is H, Z or 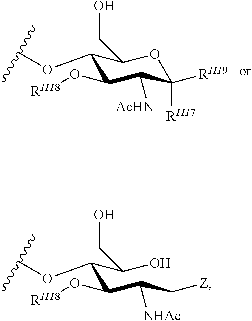
R^{III3} and R^{III8} are both H;
wherein R^{III6} is H or Z or 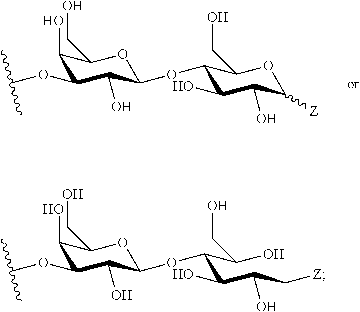
wherein R^{III7} is H or Z;
wherein when R^{III6} is H, then R^{III7}is Z and when R^{III6} is not H, then R^{III7} is H;
wherein R^{III9} is H or Z or 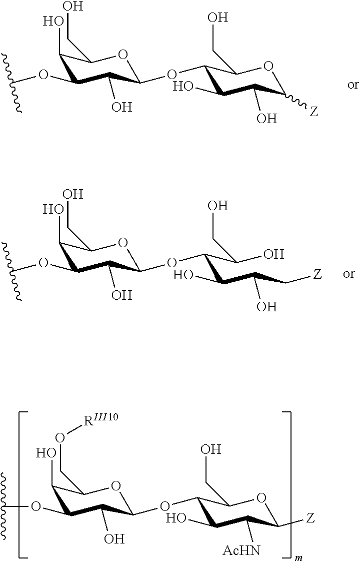
wherein m is 1 to 3;
wherein when R^{III9} is 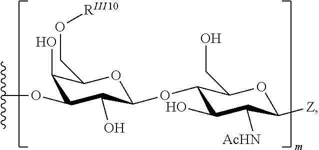
then R^{III3}, R^{III4}, R^{III5} and R^{III8} are H;
wherein R^{III10} is H or 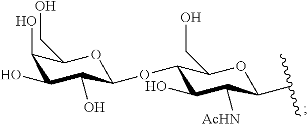
wherein formula (IV) is 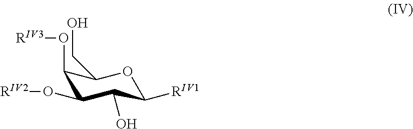
wherein R^{IV1} is 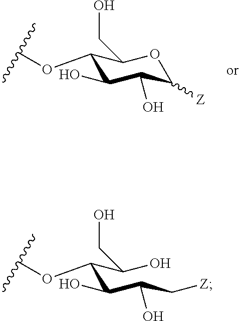
wherein R^{IV2} and R^{IV4} are independently H or 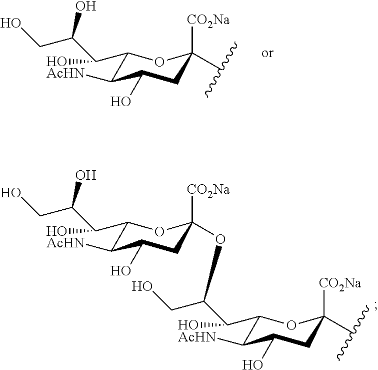
wherein R^{IV3} is H or 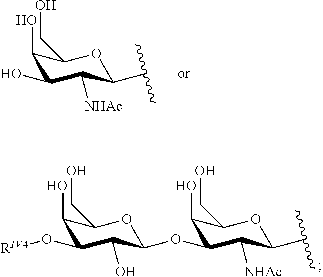
wherein (preferably) when said compound is a compound of formula (IV), then said linker Z is not
—N(R^{a})-A-B-CH₂—(CH₂)_{q}—SH, wherein
R^{a} is H, C₁-C₄-alkyl, C₁-C₄-alkoxy, CH₂C₆H₅, CH₂CH₂C₆H₅, OCH₂C₆H₅, or OCH₂CH₂C₆H₅;
A is C₁₋₇alkylene, C₁-C₇-alkoxy, C₁₋₄alkylene-(OCH₂CH₂)ᵣO—C₁-₄alkylene or C₁-C₇-alkoxy-R^{b}, wherein R^{b} is an optionally substituted aryl or an optionally substituted heteroaryl, and wherein r is 0 to 6, preferably r is 1, 2 or 3, and further preferably r is 1;
B is NHC(O), S or CH2;
q is 0 to 6, preferably q is 1, 2, 3 or 4, and further preferably q is 1 or 2.

13. A pharmaceutical composition comprising a polymer according to any one of the claims 1 to 9, or a compound according to any one of claims 10 to 13.

14. A polymer according to any one of the claims 1 to 9, a compound according to any one of the claims 10 to 12, or a pharmaceutical composition of claim 13 for use in a method of treating a disease or disorder, wherein said disease or disorder is selected from a bacterial infection, an agglutination disorder or a disorder caused by immune complex deposit-forming immunoglobulins, wherein preferably said bacterial infection is caused by Shigella, preferably by S. dysenteriae, Escherichia coli, Vibrio cholerae, Clostridium difficile, Clostridium botulinum, Clostridium tetani, Bordetella pertussis; and wherein preferably said agglutination disorder is caused by anti-A agglutinins, anti-B agglutinins, anti-I system agglutinins, anti-P system agglutinins, or anti-Tn and anti-sialyl-Tn agluttinins; and wherein preferably said disorder caused by immune complex deposit-forming immunoglobulins is caused by immunoglobulins binding to the Tn and sialyl-Tn antigen on other immunoglobulins, preferably selected from IgG, IgA, IgM.

15. A compound according to any one of claims 10 to 12, or a polymer according to any one of claims 1 to 9, or a pharmaceutical composition of claim 13 for use in a method of diagnosis of a disease or disorder, wherein said disease or disorder is selected from a bacterial infection, an agglutination disorder or a disorder caused by immune complex deposit-forming immunoglobulins, wherein preferably said disease or disorder is selected from shigellosis, bacillary dysentery, Marlow syndrome, hemolytic-uremic syndrome (HUS), travelers’ diarrhea, cholera, Clostridium difficile infection, botulinism, tetanus, pertussis or whooping cough, ABH-incompatible transplantation/transfusion, cold agluttinin disease, paroxysmal cold hemoglobinuria, Tn polyagglutinability syndrome, IgA nephropathy (also known as IgA nephritis or Berger disease or synpharyngitic glomerulonephritis) or IgA vasculitis (also known as Henoch Schönlein Purpura (HSP).
